(19) United States
(12) Patent Application Publication
(10) Pub. No.: US 2010/0120858 A1
(43) Pub. Date: May 13, 2010

(54) Piperidine Derivatives

(75) Inventors: Bradley W. Caprathe, Livonia, MI (US); Rocco D. Gogliotti, Kingston Springs, TN (US); Rex A. Jennings, Voluntown, CT (US); Lloyd J. Simons, Mattawan, MI (US)

Correspondence Address: PFIZER INC.;PATENT DEPARTMENT, Bld 114 M/S 114, EASTERN POINT ROAD, GROTON, CT, 06340, US

(73) Assignee: Pfizer Inc.

(21) Appl. No.: 12/438,213

(22) PCT Filed: Aug. 13, 2007
(86) PCT No.: PCT/IB07/02445

§ 371 Date: Jan. 7, 2010

Related U.S. Application Data
(60) Provisional application No. 60/839,641, filed on Aug. 23, 2006.

(51) Int. Cl. A61K 31/4545; C07D 401/02; A61P 19/00; A61K 31/445; C07D 211/20

(52) U.S. Cl. 514/318; 546/194; 546/248; 514/315

(57) ABSTRACT

The invention relates to compounds of Formula (I)

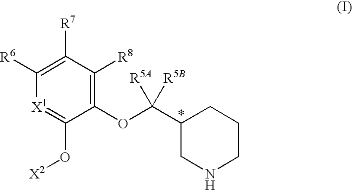

and pharmaceutically acceptable acid addition salts thereof, wherein *, X¹, X², R^{5A}, R^{5B}, R⁶, R⁷, and R⁸ are as defined in the specification; pharmaceutical compositions; therapeutic combinations; and methods of treating diseases and disorders.

### FIELD OF THE INVENTION

The present invention relates to compounds, pharmaceutical compositions, therapeutic combinations, uses, and methods of therapeutic treatment.

### BACKGROUND

Drugs that inhibit the reuptake of the monoamine neurotransmitter norepinephrine (also known as noradrenaline) or serotonin from a synaptic cleft into neurons are useful for treating diseases and disorders mediated by the reuptake. These diseases and disorders include depression, generalized anxiety disorder, attention deficit hyperactivity disorder (ADHD), fibromyalgia, neuropathic pain, urinary incontinence, and schizophrenia. Atomoxetine is a norepinephrine reuptake inhibitor that is approved in the United States for treating ADHD. Amitriptyline, venlafaxine, duloxetine, and milnacipran are dual norepinephrine and serotonin reuptake inhibitors that have successfully been used in clinical trials to treat fibromyalgia, which is one of the most common diagnoses made in rheumatological practice. Reuptake inhibitors have also been shown in human clinical trials to be efficacious for treating neuropathic pain, urinary incontinence, generalized anxiety disorder, depression, and schizophrenia. There is a need in the pharmaceutical and veterinary arts for new compounds that treat such diseases and disorders.

### SUMMARY OF THE INVENTION

An embodiment of the invention is a compound of Formula (I)

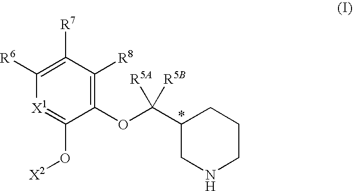

or a pharmaceutically acceptable acid addition salt thereof,

wherein:
* indicates a first chiral carbon atom;
R^{5A} and R^{5B} independently are H, (C₁-C₄)alkyl, phenyl, or pyridyl;
X¹ is N or C—R¹;
R¹ is H or halo;
R⁶ independently is H, halo, (C₁-C₄)alkyl, or —O(C₁-C₄)alkyl;
R⁷ and R⁸ independently are H or F;
X² is

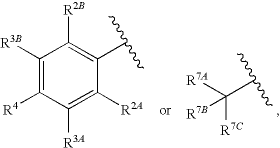

R^{2A}, R^{2B}, R^{3A}, R^{3B}, and R⁴ independently are H, halo, (C₁-C₄)alkyl, —CN, or —O(C₁-C₄)alkyl, or R^{2A} and R^{3A}, or R^{3A} and R⁴, may be taken together with the carbons to which they are attached to form a 1,2-cyclopentenylene or 1,2-cyclohexenylene;
R^{7A} and R^{7B} independently are H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl, or R^{7A} and R^{7B} optionally may be taken together with the carbon to which they are attached to form a (C₃-C₆)cycloalkyl;
R^{7C} is H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl;
each of the 1,2-cyclopentenylene, 1,2-cyclohexenylene, (C₁-C₄)alkylene, (C₁-C₄)alkyl, (C₃-C₆)cycloalkyl, and —O(C₁-C₄)alkyl independently is unsubstituted or substituted with from 1 to 5 substituents R^{S};
each phenyl independently is unsubstituted or substituted with from 1 to 5 substituents R^{T};
each pyridyl is unsubstituted or substituted with from 1 to 4 substituents R^{T};
each R^{S} independently is F, —CH₃, —CF₃, —CN, —OCH₃, ═O, —NH₂, —N(H)CH₃, or —N(CH₃)₂;
each R^{T} independently is F, Cl, —CH₃, —CF₃, —CN, —OCH₃, —OCH₂CH₃, —NH₂, or —N(H)CH₃; and
wherein at least one of R¹, R^{2A}, R^{3A}, R^{3B}, R⁴, R⁶, R⁷, and R⁸, is not H; and X² is not —CH₃.

In some embodiments, X² is

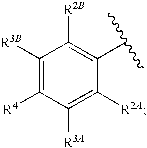

one of R^{2A}, R^{2B}, R^{3A}, R^{3B}, and R⁴ is halo, (C₁-C₄)alkyl, or —O(C₁-C₄)alkyl; and the remainder of R^{2A}, R^{2B}, R^{3A}, R^{3B}, and R⁴ independently are H, halo, (C₁-C₄)alkyl, or —O(C₁-C₄)alkyl.

In some embodiments X² is

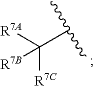

R^{7A}, R^{7B}, and R^{7C} independently are H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl; and X² is not —CH₃.
In some embodiments X² is

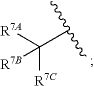

R^{7A} and R^{7B} are taken together with the carbon to which they are attached to form a (C₃-C₆)cycloalkyl; and R^{7C} is H.

In some embodiments, X¹ is N and R⁶ is H or —CH₃.

In some embodiments, X¹ is C—R¹; R¹ is H or F; and R⁶ is H, F, Cl, —CH₃, —CF₃, —OCF₃, or —OCH₃.

In some embodiments, R^{5A} and R^{5B} are each H.

In some embodiments, R^{5A} is unsubstituted (C₁-C₄)alkyl, unsubstituted phenyl, or unsubstituted pyridyl; R^{5B} is H; and the carbon to which R^{5A} and R^{5B} are attached is a second chiral carbon atom.

In some embodiments, the stereochemistry is (S) at the first chiral carbon atom.

In some embodiments is a compound of Formula (I) selected from the group consisting of:
(S)-2-(2-methoxy-4-methyl-phenoxy)-6-methyl-3-(piperidin-3-ylmethoxy)-pyridine;
(S)-2-(4-chloro-2-methoxy-phenoxy)-6-methyl-3-(piperidin-3-ylmethoxy)-pyridine;
(S)-2-(2-chloro-4-methyl-phenoxy)-6-methyl-3-(piperidin-3-ylmethoxy)-pyridine;
(S)-2-(4-chloro-2-fluoro-phenoxy)-6-methyl-3-(piperidin-3-ylmethoxy)-pyridine;
(S)-2-(2,4-difluoro-phenoxy)-6-methyl-3-(piperidin-3-ylmethoxy)-pyridine;
(S)-6-methyl-3-(piperidin-3-ylmethoxy)-2-p-tolyloxy-pyridine;
(S)-2-(4-ethyl-2-methoxy-phenoxy)-6-methyl-3-(piperidin-3-ylmethoxy)-pyridine;
(S)-2-(4-chloro-phenoxy)-6-methyl-3-(piperidin-3-ylmethoxy)-pyridine;
(S)-2-(3-chloro-phenoxy)-6-methyl-3-(piperidin-3-ylmethoxy)-pyridine;
(S)-2-(3,4-dichloro-phenoxy)-6-methyl-3-(piperidin-3-ylmethoxy)-pyridine;
(S)-2-(2,4-dichloro-phenoxy)-6-methyl-3-(piperidin-3-ylmethoxy)-pyridine;
(S)-2-(2-chloro-4-fluoro-phenoxy)-6-methyl-3-(piperidin-3-ylmethoxy)-pyridine;
(S)-2-(4-chloro-3-fluoro-phenoxy)-6-methyl-3-(piperidin-3-ylmethoxy)-pyridine;
(S)-3-[4-chloro-2-(4-chloro-2-fluoro-phenoxy)-phenoxymethyl]-piperidine;
(S)-3-[4-chloro-2-(4-chloro-2-methoxy-phenoxy)-phenoxymethyl]-piperidine;
(S)-3-[2-(4-chloro-2-methoxy-phenoxy)-4-trifluoromethyl-phenoxymethyl]-piperidine; and
(S)-3-[4-chloro-2-(2-fluoro-6-methoxy-phenoxy)-phenoxymethyl]-piperidine;

or a pharmaceutically acceptable acid addition salt thereof.

In some embodiments is a compound of Formula (I) selected from the group consisting of:
(S)-2-(4-fluoro-phenoxy)-6-methyl-3-(piperidin-3-ylmethoxy)-pyridine;
(S)-2-(4-fluoro-phenoxy)-3-(piperidin-3-ylmethoxy)-pyridine;
(S)-2-(2,4-difluoro-phenoxy)-3-(piperidin-3-ylmethoxy)-pyridine;
(S)-2-(3-chloro-phenoxy)-3-(piperidin-3-ylmethoxy)-pyridine;
(S)-2-(3,4-difluoro-phenoxy)-3-(piperidin-3-ylmethoxy)-pyridine;
(S)-2-(2-chloro-4-fluoro-phenoxy)-3-(piperidin-3-ylmethoxy)-pyridine;
(S)-2-(2,6-difluoro-phenoxy)-3-(piperidin-3-ylmethoxy)-pyridine;
(S,S)-2-phenoxy-3-(1-piperidin-3-yl-propoxy)-pyridine;
(S)-2-ethoxy-3-(phenyl-piperidin-3-yl-methoxy)-pyridine, stereoisomer A;
(S)-2-phenoxy-3-(piperidin-3-ylmethoxy)-pyridine;
(S)-6-methyl-2-phenoxy-3-(piperidin-3-ylmethoxy)-pyridine;
(S)-3-(2-phenoxy-phenoxymethyl)-piperidine;
(S)-3-(4-fluoro-2-phenoxy-phenoxymethyl)-piperidine;
(S)-3-[(S)-1-(2-benzyloxy-phenoxy)-ethyl]-piperidine;
(S)-3-[(S)-1-(2-isobutoxy-phenoxy)-ethyl]-piperidine;
(S)-3-[(S)-1-(2-cyclobutylmethoxy-phenoxy)-ethyl]-piperidine;
(S)-3-[(S)-1-(2-cyclohexyloxy-phenoxy)-ethyl]-piperidine;
(S)-3-[2-fluoro-6-(4-fluoro-phenoxy)-phenoxymethyl]-piperidine;
(S)-3-[2-(3,4-difluoro-phenoxy)-6-fluoro-phenoxymethyl]-piperidine;
(S)-3-[3-fluoro-2-(4-fluoro-phenoxy)-phenoxymethyl]-piperidine;
2-[{(R)-2-fluoro-6-methoxy-phenoxy}-(S)-piperidin-3-yl-methyl]-pyridine; and
2-[(S)-piperidin-3-yl-{(R)-2-trifluoromethoxy-phenoxy}-methyl]-pyridine;

or a pharmaceutically acceptable acid addition salt thereof.

Another embodiment is a pharmaceutical composition comprising a compound of Formula (I), or a pharmaceutically acceptable acid addition salt thereof, and a pharmaceutically acceptable excipient.

Another embodiment is a use of a compound of Formula (I), or a pharmaceutically acceptable acid addition salt thereof, in the manufacture of a medicament for treating fibromyalgia; osteoarthritis or rheumatoid arthritis; or a disease or disorder selected from the group consisting of: attention deficit hyperactivity disorder; neuropathic pain; anxiety; depression; and schizophrenia.

### DETAILED DESCRIPTION OF THE INVENTION

Embodiments of the invention include compounds of Formula (I), and pharmaceutically acceptable acid addition salts thereof, pharmaceutical compositions, and methods of treating diseases and disorders. In Formula (I), the carbon to which R^{5A} and R^{5B} are attached is a second chiral carbon atom when R^{5A} and R^{5B} are different. When R^{5A} and R^{5B} are the same, the carbon to which R^{5A} and R^{5B} are attached is an achiral carbon.

In a drawing of a structure fragment, the symbol

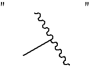

indicates a point of attachment of the fragment.

The term “halo” means F, Cl, Br, or I. In some embodiments, halo is F or Cl. In some embodiments, halo is F.

The term “(C₁-C₄)alkyl” means a straight or branched hydrocarbon chain radical of from 1 to 4 carbons. Each (C₁-C₄)alkyl independently may be unsubstituted or substituted with from 1 to 5 substituents. Each substituent independently is F, —CH₃, —CF₃, —CN, —OCH₃, ═O, —NH₂, —N(H)CH₃, or —N(CH₃)₂. Examples of unsubstituted (C₁-C₄)alkyl are methyl, ethyl, n-propyl, isopropyl, n-butyl, isobutyl, sec-butyl, and tert-butyl. Examples of substituted (C₁-C₄)alkyl are —CF₃, —CH₂OCH₃, —CF₂CF₃, isopentyl, and —CH₂CH(NH₂)CH₃. In some embodiments, (C₁-C₄)alkyl is —CH₃, —CF₃, or —CH₂CH₃.

The terms “1,2-cyclopentenylene” and “1,2-cyclohexenylene” mean carbocyclic diradicals of the formulas:

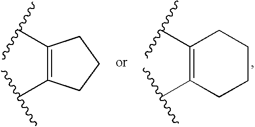

respectively.
Each 1,2-cyclopentenylene and 1,2-cyclohexenylene may be unsubstituted or substituted with from 1 to 5 substituents. Each substituent independently is F, —CH₃, —CF₃, —CN, —OCH₃, ═O, —NH₂, —N(H)CH₃, or —N(CH₃)₂. Examples of substituted 1,2-cyclopentenylene are 3-oxo-1,2-cyclopentenylene, 4-trifluoromethyl-1,2-cyclopentenylene, and 3-methoxy-1,2-cyclopentenylene. Examples of substituted 1,2-cyclohexenylene are 3,3-difluoro-1,2-cyclohexenylene, 4-methyl-1,2-cyclohexenylene, and 4-amino-4-methyl-1,2-cyclohexenylene.

The term “(C₃-C₆)cycloalkyl” means a carbocyclic radical of from 3 to 6 carbons. Each (C₃-C₆)cycloalkyl independently may be unsubstituted or substituted with from 1 to 5 substituents. Each substituent independently is F, —CH₃, —CF₃, —CN, —OCH₃, ═O, —NH₂, —N(H)CH₃, or —N(CH₃)₂. Examples of unsubstituted (C₃-C₆)cycloalkyl are cyclopropyl, cyclobutyl, cyclopentyl, and cyclohexyl. Examples of substituted (C₃-C₆)cycloalkyl are 2-methyl-cyclopropyl, cyclobutanon-3-yl (i.e., 3-oxo-cyclobutyl), 2,2,5,5-tetrafluoro-cyclopentyl, and 3-cyano-4-amino-cyclohexyl.

The term “—(C₁-C₄)alkylene-(C₃-C₆)cycloalkyl” means a radical wherein the (C₃-C₆)cycloalkyl is as defined above and is bonded to a (C₁-C₄)alkylene. A (C₁-C₄)alkylene is a straight or branched hydrocarbon chain diradical of from 1 to 4 carbons and the two radicals of the (C₁-C₄)alkylene may be at the same or different carbons of the chain. The (C₁-C₄)alkylene and the (C₃-C₆)cycloalkyl independently are unsubstituted or substituted with from 1 to 5 substituents each. Each substituent independently is F, —CH₃, —CF₃, —CN, —OCH₃, ═O, —NH₂, —N(H)CH₃, or —N(CH₃)₂. Examples of unsubstituted —(C₁-C₄)alkylene-(C₃-C₆)cycloalkyl are cyclopropylmethyl, 1-cyclobutylethyl, 2-cyclopentylpropyl, and cyclohexylmethyl. Examples of substituted —(C₁-C₄)alkylene-(C₃-C₆)cycloalkyl are 2-methyl-cyclopropylmethyl, 2-cyclobutanon-3-ylethyl (i.e., 2-(3-oxo-cyclobutyl)-ethyl), and 4-amino-cyclohexylmethyl.

The term “—(C₁-C₄)alkylene-phenyl” means a radical wherein the phenyl is bonded to a (C₁-C₄)alkylene, wherein the (C₁-C₄)alkylene is as defined above. The (C₁-C₄)alkylene and the phenyl independently are unsubstituted or substituted with from 1 to 5 substituents each. Each (C₁-C₄)alkylene substituent independently is F, —CH₃, —CF₃, —CN, —OCH₃, ═O, —NH₂, —N(H)CH₃, or —N(CH₃)₂. Each phenyl substituent independently is F, Cl, —CH₃, —CF₃, —CN, —OCH₃, —OCH₂CH₃, —NH₂, —N(H)CH₃, or —N(CH₃)₂. Examples of unsubstituted —(C₁-C₄)alkylene-phenyl are benzyl, 1- and 2-phenethyl, 3-phenylpropyl, and 4-phenylbutyl. Examples of substituted —(C₁-C₄)alkylene-phenyl are —CF₂CH₂-(2,6-difluorophenyl), 4-chloro-benzoyl, and —CH(NH₂)-(4-methoxyphenyl).

The term “—O(C₁-C₄)alkyl” means a (C₁-C₄)alkoxy radical wherein the (C₁-C₄)alkyl, a straight or branched hydrocarbon chain of from 1 to 4 carbons, is bonded to the oxygen. Each —O(C₁-C₄)alkyl independently may be unsubstituted or substituted on the hydrocarbon chain with from 1 to 5 substituents. Each substituent independently is F, —CH₃, —CF₃, —CN, —OCH₃, ═O, —NH₂, —N(H)CH₃, or —N(CH₃)₂, wherein the —OCH₃, —NH₂, —N(H)CH₃, and —N(CH₃)₂ substituents are not bonded to the carbon that is bonded to the oxygen radical Examples of unsubstituted —O(C₁-C₄)alkyl are methoxy, ethoxy, n-propoxy, isopropoxy, n-butoxy, iso-butoxy, sec-butoxy, and tert-butoxy. Examples of substituted —O(C₁-C₄)alkyl are —OCF₃, —OC(═O)CH₃, —OCH₂OCH₃, —OCF₂CF₃, isopentoxy, and —OCH₂CH(NH₂)CH₃. In some embodiments, —O(C₁-C₄)alkyl is methoxy, —OCF₃, or ethoxy. In some embodiments, each substituent on —O(C₁-C₄)alkyl independently is F, —CH₃, or —CF₃.

In some embodiments, phenyl is unsubstituted. In other embodiments, phenyl is substituted with from 1 to 3 substituents selected from the group consisting of: F, Cl, —CH₃, —CF₃, —OCH₃, and —OCH₂CH₃. Examples of substituted phenyl are 4-chlorophenyl, 2-fluoro-4-trifluoromethylphenyl, 4-methylphenyl, and 2-ethoxyphenyl.

A “pyridyl” includes pyridin-2-, -3-, and -4-yl. In some embodiments, pyridyl is unsubstituted pyridin-2-yl. In other embodiments, pyridyl is pyridin-2-yl that is substituted with from 1 to 4 substituents independently selected from the group consisting of: —CH₃, —CF₃, —OCH₃, and —OCH₂CH₃.

In some embodiments, members of the groups halo, —(C₁-C₄)alkylene-(C₃-C₆)cycloalkyl, —(C₁-C₄)alkylene-phenyl, (C₁-C₄)alkyl, phenyl, pyridyl, and —O(C₁-C₄)alkyl are selected from the particular members of those groups that are exemplified by the compounds of the Examples.

Some of the compounds and salts thereof of the invention may exist as stereoisomers, including enantiomers, diastereomers, and geometric isomers. All stereoisomers, including (R) enantiomers, (S) enantiomers, epimers, diastereomers, cis, trans, syn, anti, and mixtures thereof, including racemic (i.e., 50:50) and non-racemic (i.e., between 100:0 and 50:50) mixtures, are part of the invention. When stereochemistry of a chiral carbon atom in a compound is not specified, the stereochemistry at that chiral carbon atom may be (R), (S), or mixtures thereof.

The term “chiral carbon atom” means a carbon atom that has four different atoms or groups of atoms bonded to it.

Where a particular stereochemistry at any chiral carbon atom in a compound of Formula (I) is designated as being (S), what is meant is that the ratio of (S) stereochemistry to (R) stereochemistry at the chiral carbon is greater than 95:5. Where a particular stereochemistry at any chiral carbon atom is designated herein as being (R), what is meant is that the ratio of (R) stereochemistry to (S) stereochemistry at the chiral carbon is greater than 95:5.

The compounds and the salts thereof of the invention can be administered as solvates, including hydrates, and mixtures thereof.

The invention includes isotopically-labeled compounds of Formula (I), and pharmaceutically acceptable acid addition salts thereof. An isotopically-labeled compound of Formula (I), or a pharmaceutically acceptable acid addition salt thereof, is identical to the unlabeled compound, or the salt thereof, but for the fact that one or more atoms are replaced by an atom having an atomic mass or mass number different from the atomic mass or mass number usually found in nature (i.e., different from the naturally abundant atomic mass or mass number). Examples of contemplated isotopes include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. The isotopically-labeled compounds of Formula (I), for example those into which radioactive isotopes such as ³H and ¹⁴C are incorporated, and the salts thereof, are useful in drug and/or substrate tissue distribution assays. Tritiated, i.e., ³H and carbon-14, i.e., ¹⁴C, isotopes are particularly preferred for their ease of preparation and detectability. Further, substitution with heavier isotopes such as deuterium, e.g., ²H, may afford some therapeutic advantages resulting from greater metabolic stability, for example increased in vivo half-life or reduced dosage requirements and, hence, may be preferred in some circumstances for use in treating a disease or disorder according to a method of the invention. An isotopically-labeled compound can generally be prepared by substituting a readily available isotopically labeled reagent for a non-isotopically labeled reagent in a conventional method of preparing the compound.

Compounds of Formula (I) are capable of forming “pharmaceutically acceptable acid addition salts,” including disalts, which may be formed, for example, by contacting compounds of Formula (I) having two basic functional groups with more than one mole equivalent of a monoacid or more than one half mole equivalent of a diacid. In some embodiments, the disalts contain from 1.9 to 2.1 mole equivalents of a monoacid or from 0.95 to 1.05 mole equivalents of a diacid. Examples of suitable acids useful for forming the pharmaceutically acceptable acid addition salts can be found for example in Stahl and Wermuth, Handbook of Pharmaceutical Salts: Properties, Selection, and Use, Wiley-VCH, Weinheim, Germany (2002); and Berge et al., “Pharmaceutical Salts,” J. of Pharmaceutical Science, 1977; 66:1-19.

Examples of pharmaceutically acceptable acid addition salts of the compounds of Formula (I) include salts derived from inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include the anions acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate (methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), trifluoroacetate, and xinafoate, and the like. Also part of the invention are the salts of amino acids that include anions such as arginate, gluconate, galacturonate, and the like.

The acid addition salt of a compound of Formula (I) may be prepared using conventional methods by contacting the free base form of the compound with a sufficient amount of a desired acid to produce the salt. The free base form may be regenerated by contacting the salt with a base and isolating the free base form.

Compounds of Formula (I) having an acidic proton are capable of forming pharmaceutically acceptable base addition salts with bases such as sodium hydroxide in the case of a sodium salt. Examples of bases suitable for forming such salts are found for example in Stahl and Wermuth, supra and Berge, et al., supra.

The terms “treat,” “treating,” and “treatment” include prophylactic and palliative treatments, acute (3 months or shorter duration) and chronic treatments (more than 3 months duration), symptomatic and disease-modifying treatments.

The term “patient” means a mammal, which includes a human, dog, cat, horse, cow, pig, sheep, goat, primate, and other mammals. In some embodiments, the patient is a human. In some embodiments, the patient is a dog or cat.

The phrase “a patient in need of treatment” refers to a mammal at risk for developing a disease or disorder, or a mammal having at least one symptom thereof such as pain, having at least one sign thereof such as narrowed joint space or an abnormal biomarker, or having a pathological hallmark thereof such as nerve damage.

The term “administering” generally refers to a process of contacting a pharmaceutically active ingredient with a patient. A compound of Formula (I), or a pharmaceutically acceptable acid addition salt thereof, can be administered to a patient by injection, that is, intravenously, intramuscularly, intracutaneously, subcutaneously, intraduodenally, parentally, or intraperitoneally; by inhalation, for example, intranasally; transdermally, topically, and via implantation. In some embodiments, the compound or the salt thereof is administered orally. Administering may also be rectally, bucally, intravaginally, ocularly, or by insufflation. Administering may also be via intravenous infusion, orally, topically, intraperitoneally, intravesically, or intrathecally. Administering includes sustained- or extended-release formulations. The active ingredient can be administered to the patient at a rate determined by factors that may include, but are not limited to, the pharmacokinetic profile of the active ingredient, contraindicated drugs being present in the patient, and the side effects of the active ingredient at various concentrations, in view of the body mass (e.g., weight or body surface area) and health of the subject.

Administering a single therapeutically effective dose and administering multiple therapeutically effective doses are both part of the invention. Any therapeutically effective dose can be divided into multiple sub-therapeutically effective doses, which can be administered simultaneously or sequentially. Sequential administration of multiple sub-therapeutically effective doses is carried out such that a therapeutically effective level (e.g., blood concentration) of the active ingredient being administered is eventually achieved in the patient being treated. Determination of a suitable route and rate of administration is within the level of ordinary skill in the medical and veterinary arts.

Treatment may be evaluated using conventional patient assessment tools and diagnostic methods. Examples of these tools are the Fibromyalgia Impact Questionnaire (FIQ), Western Ontario and McMaster Universities Osteoarthritis Index (WOMAC), Lequesne's functional index, Patient Global Impression of Change (PGIC) questionnaire, Liked pain scale, and Visual Analog Scale (VAS) of pain. Examples of diagnostic methods are x-ray measurements of joint space narrowing in osteoarthritis patients and blood tests for rheumatoid factor in rheumatoid arthritis patients. It is within the ordinary skill of a physician or veterinarian to determine whether or not, and how, a particular treatment is effective.

The term “fibromyalgia” is also known as fibromyalgia syndrome. The American College of Rheumatology (ACR) 1990 classification criteria for fibromyalgia include a history of chronic, widespread pain for more than three months, and the presence of pain at 11 (or more) out of 18 tender points upon physical examination, wherein the tender points occur both above and below the waist and on both sides of the body (see e.g., Wolfe et al., Arthritis Rheum., 1990; 33:160-172). Fibromyalgia patients generally display pain perception abnormalities in the form of both allodynia (pain in response to a normally non-painful stimulus) and hyperalgesia (an increased sensitivity to a painful stimulus). The effects of fibromyalgia in a human patient may be assessed using the ACR criteria, an FIQ total score, indices of pain severity (e.g., VAS or Liked pain scales) and interference, the number of tender points, or a pain threshold assessment.

Although chronic, widespread pain is a hallmark symptom of fibromyalgia, patients typically also exhibit other symptoms, including one or more of the following: fatigue, sleep disturbances, migraine or tension headaches, irritable bowel syndrome (IBS), changes in urinary frequency, morning stiffness, numbness and tingling, dysmenorrhea, multiple chemical sensitivities, difficulty concentrating, and circulatory problems that affect the small blood vessels of the skin (Raynaud's phenomenon). As with many diseases and disorders that cause chronic pain, fibromyalgia patients may also experience fibromyalgia-induced anxiety, depression, or both. Some fibromyalgia patients find that cold, damp weather, emotional stress, overexertion, and other factors exacerbate their symptoms.

Treating fibromyalgia includes treating at least one symptom associated with fibromyalgia such as pain and the other symptoms of fibromyalgia mentioned previously. Pain associated with fibromyalgia includes the chronic, widespread pain that is a hallmark of fibromyalgia and pain associated with the other symptoms of fibromyalgia. Examples of pain associated with the other symptoms of fibromyalgia are migraine, tension headache, dysmenorrhea, and visceral pain associated with IDS. In some embodiments, treating fibromyalgia means reducing the chronic, widespread pain that is a hallmark of fibromyalgia.

Treating rheumatoid arthritis (RA), an inflammatory arthritis of a joint, includes treating at least one symptom of RA or inhibiting pathological destruction of the cartilage of the joint. Examples of symptoms of RA are joint pain and swelling of the joint. Diagnosis of RA in a human patient may be made by a physician using, for example, ACR-20 criteria. In certain embodiments, treating RA means reducing pain associated with rheumatoid arthritis and includes reducing at least one of RA joint pain and referred RA pain.

Treating osteoarthritis (OA), includes treating at least one symptom of OA such as pain or inhibiting the pathological destruction of the cartilage of an OA joint. OA is a form of arthritis characterized by pathological loss of articular cartilage and hypertrophy of bone near the affected joint that progressively leads to reduction in joint motion, tenderness grating sensations in the joint, and joint pain. Diagnosis of OA in a human patient may be made by a physician using, for example, WOMAC criteria and blood tests to rule out other forms of arthritis. In certain embodiments treating OA means reducing pain associated with OA and includes reducing at least one of OA joint pain and referred OA pain.

Referred pain is pain that is perceived by a patient at a site in the patient's body that is distal from the origin of the pain.

The term “therapeutically effective amount” refers to an amount of a pharmaceutically active ingredient such as a compound of Formula (I) that is sufficient to increase the time to onset of at least one symptom in prophylactic treatment, diminish the severity of at least one symptom in palliative treatment, or inhibit the progression of a pathological effect in disease modifying treatment of a disease or disorder in a patient according to a method of the invention. For a human or other mammal, a therapeutically effective amount can be determined by a physician or veterinarian in a clinical setting in accordance with the particular disease or disorder or patient being treated. The amount will be determined by the efficacy of the particular active ingredient employed and the disease or disorder of the patient, as well as the body weight or surface area of the patient to be treated. The size of the dose also will be determined by the existence, nature, and extent of any adverse effects that accompany the administration of a particular compound to a particular patient. In determining the therapeutically effective amount of an active ingredient, the physician or veterinarian can evaluate factors such as the circulating plasma levels of the active ingredient, associated toxicities, the progression and severity of the disease or disorder, and the like. Determination of a therapeutically effective amount is within the level of ordinary skill in the medical and veterinary arts.

A “pharmaceutically active ingredient” may be referred to as an active ingredient, active component, active compound, a drug, or the like. Examples of pharmaceutically active ingredients are compounds of Formula (I), pharmaceutically acceptable acid addition salts thereof, and pharmaceutically active compounds that are not compounds of Formula (I) such as alpha-2-delta ligands and nonsteroidal anti-inflammatory drugs (NSAIDs).

In general, a therapeutically effective amount of a compound of Formula (I), or a pharmaceutically acceptable acid addition salt thereof, is from about 0.01 milligrams of the compound or salt per kilogram of patient body weight (mg/kg) to about 30 mg/kg for a patient of 70 kg body weight. In some embodiments, the daily dose range is from about 0.1 mg/kg to about 10 mg/kg. The daily dosages, however, may be varied depending upon the requirements of the patient, the severity of the disease or disorder being treated, and the particular active ingredient being employed.

Treatment may be initiated with smaller dosages, which may be less than the optimum dose and may be a sub-therapeutic dose. For example, a starting daily dosage may be from about 0.001 mg/kg to about 10 mg/kg. Thereafter, the dosage is increased by small increments until the optimum effect under the circumstances is reached, usually reaching from about 0.01 mg/kg to about 30 mg/kg for a patient of 70 kg body weight. For convenience, the total daily dosage may be divided and administered in portions during the day, if desired.

The term “pharmaceutical composition” refers to a composition suitable for administering to a patient in medical or veterinary use according to a treatment method of the invention. In some embodiments, a pharmaceutical composition of the invention comprises a compound of Formula (I), or a pharmaceutically acceptable acid addition salt thereof, and a pharmaceutically acceptable excipient. Pharmaceutical compositions include homogeneous and heterogeneous mixtures. The pharmaceutical compositions include the formulation of an invention compound or salt thereof, with encapsulating material (e.g., capsule shell) as an excipient, thereby providing a capsule in which the compound or salt thereof, with or without other excipients, is surrounded by, and in association with, the encapsulating material.

A pharmaceutical composition of the invention can be a solid or liquid form preparation and may comprise one pharmaceutically acceptable excipient or more than one. Solid form preparations include tablets, pills, capsules, lozenges, cachets, powders, suppositories, and dispersible granules. Liquid form preparations include solutions, suspensions, and emulsions. The pharmaceutical composition includes sustained- or extended-release formulations. The pharmaceutical composition may be in the form of a syrup, an elixir, a suspension, a powder, a granule, a tablet, a capsule, a lozenge, a troche, an aqueous solution, a cream, an ointment, a lotion, a gel, an emulsion, a patch, or the like. Accordingly, there are a variety of suitable formulations of pharmaceutical compositions of the invention. In some embodiments, the pharmaceutical composition is a tablet or capsule. In some embodiments, the pharmaceutical composition is suitable for topical administration. It is within the ordinary skill in the art to prepare pharmaceutical compositions of the invention.

The term “pharmaceutically acceptable excipient” refers to any component of a pharmaceutical composition that is not an invention compound, or salt thereof, or, in the case of a combination of the invention, is not another pharmaceutically active component of a pharmaceutical co-composition. Each excipient is independently selected. Examples of the excipients include pharmaceutically acceptable diluents, carriers, stabilizers, and other components such as capsule shells, for example gelatin capsule shells.

The pharmaceutically acceptable excipient can be, for example, a solid or liquid carrier, diluent, flavoring agent, binder, preservative, tablet disintegrating agent, colorant, flavor, taste-masking agent, stabilizer, thickening agent, or an encapsulating material such as a gelatin capsule. Selection of pharmaceutically acceptable excipients is determined in part by the particular active ingredient and route of administration, as well as by the particular method used to administer the active ingredient. (see, e.g., Remington: The Science and Practice of Pharmacy, 20th ed., Gennaro et al. Eds., Lippincott Williams and Wilkins, 2000).

In powder form preparations of the invention pharmaceutical composition, the excipient may be a finely divided solid, which is in a mixture with a finely divided active component. In tablets, the active component is mixed with an excipient having the necessary binding properties in suitable proportions and compacted in a desired shape and size. The powders and tablets typically contain from 1% to 95% weight/weight (w/w) of the active ingredient. In some embodiments, the active ingredient ranges from 5% to 70% (w/w). Examples of suitable excipients are magnesium carbonate, magnesium stearate, talc, sugar, lactose, pectin, dextrin, starch, gelatin, tragacanth, methylcellulose, sodium carboxymethylcellulose, a low melting wax, and cocoa butter.

For preparing suppositories, a low melting wax, such as a mixture of fatty acid glycerides or cocoa butter, is first melted and the active ingredient is dispersed homogeneously therein, such as by stirring. The molten homogeneous mixture is then poured into convenient sized molds, allowed to cool and solidify.

Liquid form preparations of the invention pharmaceutical composition include water or water/propylene glycol solutions, wherein the excipients are water or water and propylene glycol. For parenteral injection, liquid form preparations can be formulated as solutions in aqueous polyethylene glycol. Aqueous solutions suitable for oral use can be prepared by dissolving the active ingredient in water and adding suitable excipients such as colorants, flavors, taste-masking agents, stabilizers, and thickening agents as desired. Aqueous suspensions suitable for oral use can be made by dispersing a finely divided active ingredient in water with a viscous excipient such as natural or synthetic gums, resins, methylcellulose, sodium carboxymethylcellulose, and other suspending agents.

Pharmaceutical compositions suitable for parenteral administration, such as, for example, by intravenous, intramuscular, intradermal, and subcutaneous routes, may be prepared as solutions, including aqueous and non-aqueous, isotonic sterile injection solutions, which can contain antioxidants, buffers, bacteriostats, and solutes that render the formulation isotonic with the blood of the intended recipient, or as aqueous and nonaqueous sterile suspensions that can include suspending agents, solubilizers, thickening agents, stabilizers, and preservatives. The formulations can be presented in unit-dose or multi-dose sealed containers, such as ampules and vials. Solutions and suspensions for injection can be prepared from, for example, sterile powders, granules, or tablets.

Also included in the invention pharmaceutical composition are solid form preparations that are intended to be converted shortly before use to liquid form preparations for oral or parenteral administration. Such liquid forms include solutions, suspensions, and emulsions. These preparations may contain, in addition to the active ingredients, one or more excipients such as colorants, flavors, stabilizers, buffers, artificial and natural sweeteners, dispersants, thickeners, and solubilizing agents.

Other embodiments include pharmaceutical compositions that are aerosol formulations suitable for administration via inhalation. A pharmaceutically active ingredient, alone or in combination with other suitable components such as excipients or other pharmaceutically active ingredients, can be made into aerosol formulations (i.e., they can be “nebulized”) using conventional procedures. The aerosol formulations can be placed into pressurized acceptable propellants, such as dichlorodifluoromethane, propane, nitrogen, and the like.

In veterinary use, a composition for dogs or cats may comprise an ingestible liquid peroral dosage form such as a solution, suspension, emulsion, inverse emulsion, elixir, extract, tincture, or concentrate. Any of these liquid dosage forms may be formulated to be administered directly to the dog or cat (e.g., by injection or oral gavage) or indirectly, e.g., added to the food or drinking water of the dog or cat. A concentrate liquid form may be formulated for dissolution in a given amount of water, from which resulting solution a measured aliquot amount may be withdrawn for administration directly or indirectly to the dog or cat.

A pharmaceutical composition of the invention is preferably in a unit dosage form. In a unit dosage form, the composition is subdivided into unit doses containing appropriate quantities of the active ingredient(s). The unit dosage form can be a packaged preparation, the package containing discrete quantities of composition, such as packeted tablets, capsules, and powders in vials or ampules. Also, the unit dosage form can be, for example, a capsule, tablet, pill, cachet, or lozenge itself, or it can be the appropriate number of any of these in packaged form.

The quantity of an active ingredient in a unit dose composition may be varied or adjusted according to the particular application contemplated and the potency of the active ingredient. In some embodiments, the quantity is from 0.1 mg to 1000 mg. The composition can, if desired, also contain other compatible active ingredients as described herein for an invention combination.

The pharmaceutical compositions may be prepared according to processes known to one of ordinary skill in the art. A method for preparing a pharmaceutical tablet composition is provided in Tablet Formulation Example 1.

### Tablet Formulation Example 1

Tablet Formulation

| Ingredient | Amount |
|---|---|
| A compound of Formula (I) | 50 mg |
| Lactose | 80 mg |
| Cornstarch (for mix) | 10 mg |
| Cornstarch (for paste) | 8 mg |
| Magnesium stearate (1%) | 2 mg |
| Total weight | 150 mg |

A compound of Formula (I) (or a pharmaceutically acceptable acid addition salt thereof) is mixed with lactose and cornstarch (for mix) and blended to uniformity to a mixed powder. Cornstarch (for paste) is suspended in 6 mL of water and heated with stirring to form a paste. The paste is added to the mixed powder, and the resulting mixture is granulated. The wet granules are passed through a No. 8 hand screen and dried at 50° C. The mixture is lubricated with 1% magnesium stearate, and then compressed into a tablet. Such tablets can be administered to a patient at the rate of from 1 to 4 each day for treatment of a disease or disorder according to a method of the invention.

Another embodiment is a compound of Formula (Ia)

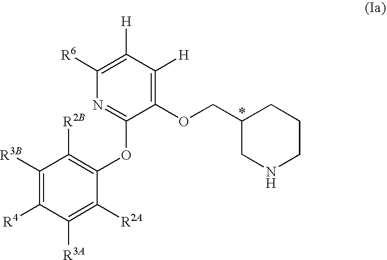

or a pharmaceutically acceptable acid addition salt thereof, wherein *, R^{2A}, R^{2B}, R^{3A}, R^{3B}, R⁴, and R⁶ are as defined herein for Formula (I).

Another embodiment is a compound of Formula (Ib)

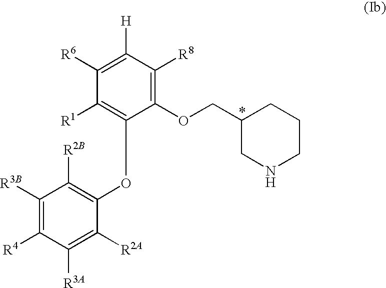

or a pharmaceutically acceptable acid addition salt thereof, wherein *, R¹, R^{2A}, R^{2B}, R^{3A}, R^{3B}, R⁴, R⁶, and R⁸ are as defined herein for Formula (I).

Another embodiment is a compound of Formula (Ic)

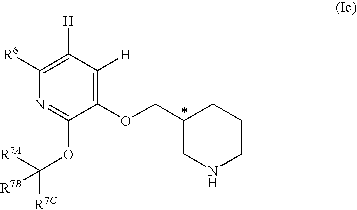

or a pharmaceutically acceptable acid addition salt thereof, wherein *, R⁶, R^{7A}, R^{7B}, and R^{7C} are as defined herein for Formula (I).

Another embodiment is a compound of Formula (Id)

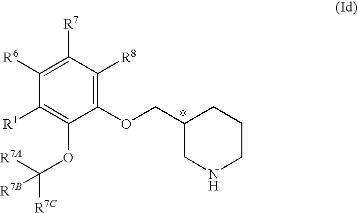

or a pharmaceutically acceptable acid addition salt thereof, wherein *, R⁶, R⁷, R^{7A}, R^{7B}, R^{7C}, and R⁸ are as defined herein for Formula (I).

Another embodiment is a compound of Formula (Ie)

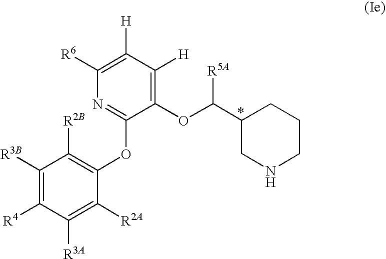

or a pharmaceutically acceptable acid addition salt thereof, wherein *, R^{2A}, R^{2B}, R^{3A}, R^{3B}, R⁴, R^{5A}, and R⁶ are as defined herein for Formula (I).

Another embodiment is a compound of Formula (If)

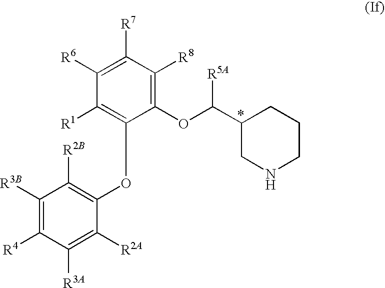

or a pharmaceutically acceptable acid addition salt thereof, wherein *, R¹, R^{2A}, R^{2B}, R^{3A}, R^{3B}, R⁴, R^{5A}, R⁶, R⁷, and R⁸ are as defined herein for Formula (I).

Another embodiment is a compound of Formula (Ig)

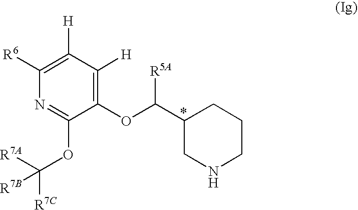

or a pharmaceutically acceptable acid addition salt thereof, wherein *, R^{5A}, R⁶, R^{7A}, R^{7B}, and R^{7C} are as defined herein for Formula (I).

Another embodiment is a compound of Formula (Ih)

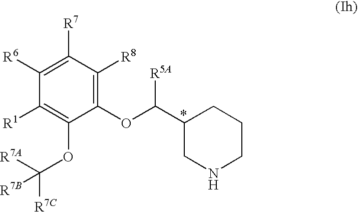

or a pharmaceutically acceptable acid addition salt thereof, wherein *, R^{5A}, R⁶, R⁷, R^{7A}, R^{7B}, R^{7C}, and R⁸ are as defined herein for Formula (I).

Another embodiment is a compound of Formula (Ii)

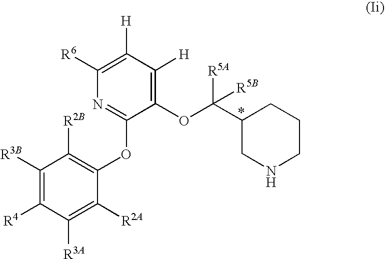

or a pharmaceutically acceptable acid addition salt thereof, wherein *, R^{2A}, R^{2B}, R^{3A}, R^{3B}, R⁴, R^{5A}, R^{5B}, and R⁶ are as defined herein for Formula (I).

Another embodiment is a compound of Formula (Ij)

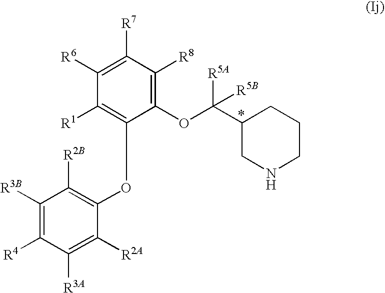

or a pharmaceutically acceptable acid addition salt thereof, wherein *, R¹, R^{2A}, R^{2B}, R^{3A}, R^{3B}, R⁴, R^{5A}, R^{5B}, R⁶, R⁷, and R⁸ are as defined herein for Formula (I).

Another embodiment is a compound of Formula (Ik)

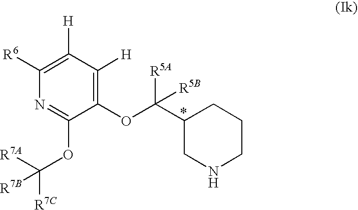

or a pharmaceutically acceptable acid addition salt thereof, wherein *, R^{5A}, R^{5B}, R⁶, R^{7A}, R^{7B}, and R^{7C} are as defined herein for Formula (I).

Another embodiment is a compound of Formula (IL)

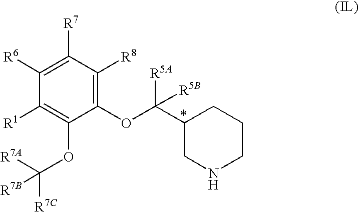

or a pharmaceutically acceptable acid addition salt thereof, wherein *, R^{5A}, R^{5B}, R⁶, R⁷, R^{7A}, R^{7B}, R^{7C}, and R⁸ are as defined herein for Formula (I).

In some embodiments, X¹ is C—R¹, wherein R¹ is H or F, and R⁶ independently is H, halo, (C₁-C₄)alkyl, or —O(C₁-C₄)alkyl. In some embodiments, R¹ is H. In other embodiments, R¹ is F. In some embodiments, R⁶ is H; in other embodiments, R⁶ is F.

In other embodiments, X¹ is N, and R⁶ independently is H or (C₁-C₄)alkyl. In other embodiments, X¹ is N and R⁶ independently is H. In other embodiments, X¹ is N and R⁶ independently is —CH₃. In other embodiments, X¹ is N, and R⁶ independently is —O(C₁-C₄)alkyl.

In some embodiments, R⁶ is H. In other embodiments, R⁶ is halo. In other embodiments, R⁶ is F or Cl. In other embodiments, R⁶ is (C₁-C₄)alkyl. In other embodiments, R⁶ is —CH₃. In other embodiments, R⁶ is —CF₃. In other embodiments, R⁶ is —O(C₁-C₄)alkyl. In other embodiments, R⁶ is —OCH₃. In other embodiments, R⁶ is —OCF₃.

In other embodiments, R^{5A} and R^{5B} are each H. In some embodiments, R^{5A} and R^{5B} are each —CH₃ or —CH₂CH₃. In some embodiments, R^{5A} is (C₁-C₄)alkyl and R^{5B} is H. In some embodiments, R^{5A} is phenyl and R^{5B} is H. In some embodiments, R^{5A} is pyridyl and R^{5B} is H.

In other embodiments, at least one of R^{2A}, R^{2B}, R^{3A}, R^{3B}, and R⁴ is not H. In other embodiments, at least one of R¹, R⁶, R⁷, and R⁸ is not H. In other embodiments, R⁶ is not H. In some embodiments, at least one of R¹, R^{2A}, R^{2B}, R^{3A}, R^{3B}, R⁴, R⁶, R⁷, and R⁸ is not H and R^{5A} is not H.

In some embodiments, one of R^{2A}, R^{2B}, R^{3A}, R^{3B}, and R⁴ is halo, (C₁-C₄)alkyl, or —O(C₁-C₄)alkyl, and the remainder of R^{2A}, R^{2B}, R^{3A}, R^{3B}, and R⁴ independently are H, halo, (C₁-C₄)alkyl, or —O(C₁-C₄)alkyl.

In some embodiments, only one of R^{2A}, R^{2B}, R^{3A}, R^{3B}, and R⁴ is halo, (C₁-C₄)alkyl, or —O(C₁-C₄)alkyl, and the remainder of R^{2A}, R^{2B}, R^{3A}, R^{3B}, and R⁴ are each H. In some embodiments, two of R^{2A}, R^{2B}, R^{3A}, R^{3B}, and R⁴ independently are halo, (C₁-C₄)alkyl, or —O(C₁-C₄)alkyl, and the remainder of R^{2A}, R^{2B}, R^{3A}, R^{3B}, and R⁴ are H.

In some embodiments, three of R^{2A}, R^{2B}, R^{3A}, R^{3B}, and R⁴ independently are halo, (C₁-C₄)alkyl, or —O(C₁-C₄)alkyl, and the remainder of R^{2A}, R^{2B}, R^{3A}, R^{3B}, and R⁴ are each H.

In some embodiments, at least one of R^{2A}, R^{2B}, R^{3A}, R^{3B}, and R⁴ independently is halo. In other embodiments, at least one of R^{2A}, R^{2B}, R^{3A}, R^{3B}, and R⁴ independently is F or Cl. In other embodiments, at least one of R^{2A}, R^{2B}, R^{3A}, R^{3B}, and R⁴ independently is (C₁-C₄)alkyl. In other embodiments, at least one of R^{2A}, R^{2B}, R^{3A}, R^{3B}, and R⁴ independently is —CH₃ or —CF₃. In other embodiments, at least one of R^{2A}, R^{2B}, R^{3A}, R^{3B}, and R⁴ independently is —O(C₁-C₄)alkyl. In other embodiments, at least one of R^{2A}, R^{2B}, R^{3A}, R^{3B}, and R⁴ independently is —OCH₃, —OCF₃, or —OCH₂CH₃.

In some embodiments, either R^{2A} and R^{2B} are each H; R^{2A} is —CH₃ and R^{2B} is H, F, Cl, —CH₃, —OCH₃, or —OCH₂CH₃; R^{2A} is —OCH₃ or —OCH₂CH₃ and R^{2B} is H, F, or Cl; R^{2A} is Cl and R^{2B} is H, F, or Cl; R^{2A} is F and R^{2B} is H or F; R^{3A} and R^{3B} independently are H, F, or Cl; or R⁴ is H, F, Cl, —CH₃, —OCH₃, or —OCH₂CH₃.

In some embodiments, R^{7A} is H, (C₁-C₄)alkyl, (C₃-C₆)cycloalkyl, or phenyl; R^{7B} is H, (C₁-C₄)alkyl, (C₃-C₆)cycloalkyl, or phenyl; and R^{7C} is H. In some embodiments, R^{7A} is (C₁-C₄)alkyl, and R^{7B} and R^{7C} each are H. In some embodiments, R^{7A} is (C₃-C₆)cycloalkyl and R^{7B} and R^{7C} each are H. In some embodiments, R^{7A} and R^{7B} are taken together to form (C₃-C₆)cycloalkyl and R^{7C} is H. In some embodiments, one of R^{7A}, R^{7B}, and R^{7C} is F and the remainder of R^{7A}, R^{7B}, and R^{7C} independently are each H or F.

In some embodiments, at least one unsubstituted —(C₁-C₄)alkylene-(C₃-C₆)cycloalkyl, —(C₁-C₄)alkylene-phenyl, (C₁-C₄)alkyl, phenyl, pyridyl, or —O(C₁-C₄)alkyl is present in a compound of Formula (I).

In some embodiments, at least one substituted —(C₁-C₄)alkylene-(C₃-C₆)cycloalkyl, —(C₁-C₄)alkylene-phenyl, (C₁-C₄)alkyl, phenyl, pyridyl, or —O(C₁-C₄)alkyl is present in a compound of Formula (I).

In some embodiments, each R^{S} independently is F, —CH₃, —CF₃, —OCH₃, ═O, or —N(CH₃)₂. In some embodiments, each R^{S} independently is F, —CH₃, —CF₃, or —OCH₃.

In some embodiments, each R^{T} independently is F, Cl, —CH₃, —CF₃, —OCH₃, or —OCH₂CH₃.

In some embodiments, the first chiral carbon has (S) stereochemistry. In some embodiments, the first chiral carbon has (R) stereochemistry. In some embodiments, the stereochemistry of the first and second chiral carbons is (S,R); in other embodiments (R,S); in still other embodiments (S,S); and in still other embodiments (R,R), respectively.

Relative amounts of the (S) and (R) stereochemistry may be determined by conventional means such as ¹H-nuclear magnetic resonance using a chiral shift reagent such as europium tris[3-(heptafluoropropylhydroxymethylene)-(+)-camphorate, enantioselective high performance liquid chromatography (HPLC) using an ultraviolet (UV) detector, polarimetry in conjunction with UV spectroscopy, and circular dichroism spectroscopy in conjunction with ultraviolet spectroscopy. In some embodiments, the relative amounts are determined by HPLC by adapting a procedure for the separation of enantiomers of reboxetine as described in Ohman, D., et al., Journal of Chromatography A, 2002; 947(2):247-254; Ficarra, R. et al., Chromatographia, 2001; 53 (5/6):261-265; or Walters, R. et al., Journal of Chromatography A, 1998; 828 (1/2):167-176.

Another embodiment is a package containing: (i) a pharmaceutical composition comprising a compound of Formula (I), or a pharmaceutically acceptable acid addition salt thereof, and a pharmaceutically acceptable excipient; and (ii) instructions for using the pharmaceutical composition to treat according to a method of the invention a disease or disorder in a patient in need of such treatment.

Another embodiment is a method of treating a norepinephrine-, serotonin-, or norepinephrine- and serotonin-mediated disease or disorder, the method comprising administering to a patient in need of such treatment a therapeutically effective amount of a compound of Formula (I), or a pharmaceutically acceptable acid addition salt thereof. The invention, however, is not bound by any theory of a biological mechanism for how the compound of Formula (I), or the salt thereof, may in fact achieve a desired therapeutic effect in a patient.

Another embodiment is a method of treating fibromyalgia, the method comprising administering to a patient in need of such treatment a therapeutically effective amount of a compound of Formula (I), or a pharmaceutically acceptable acid addition salt thereof.

Another embodiment is a method of treating osteoarthritis or rheumatoid arthritis, the method comprising administering to a patient in need of such treatment a therapeutically effective amount of a compound of Formula (I), or a pharmaceutically acceptable acid addition salt thereof.

Another embodiment is a method of treating a disease or disorder selected from the group consisting of: attention deficit hyperactivity disorder; neuropathic pain; anxiety; depression; and schizophrenia, the method comprising administering to a patient in need of such treatment a therapeutically effective amount of a compound of Formula (I), or a pharmaceutically acceptable acid addition salt thereof.

Another embodiment is a use of a compound of Formula (I), or a pharmaceutically acceptable acid addition salt thereof, in the manufacture of a medicament for treating a norepinephrine-, serotonin-, or norepinephrine- and serotonin-mediated disease or disorder in a patient.

An example of a norepinephrine-, serotonin-, or norepinephrine- and serotonin-mediated disease or disorder is fibromyalgia. Other treatable diseases and disorders include single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression. Other treatable diseases and disorders include major depression, single episode depression, recurrent depression, child abuse induced depression, and postpartum depression.

Other treatable diseases and disorders include a bipolar disorder or manic depression, for example, bipolar I disorder, bipolar II disorder, and cyclothymic disorder.

Other treatable diseases and disorders include conduct disorder, ADHD, disruptive behavior disorder, behavioral disturbances associated with mental retardation, autistic disorder, and conduct disorder.

Other treatable diseases and disorders include anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders.

Other treatable diseases and disorders include borderline personality disorder, schizophrenia, and other psychotic disorders such as schizophreniform disorders. Other treatable diseases and disorders include schizoaffective disorders, delusional disorders, substance-induced psychotic disorder, brief psychotic disorders, shared psychotic disorders, psychotic disorders with delusions or hallucinations, psychotic episodes of anxiety, anxiety associated with psychosis, psychotic disorder due to a general medical condition, psychotic mood disorders such as severe major depressive disorder, mood disorders associated with psychotic disorders such as acute mania and depression associated with bipolar disorder, and mood disorders associated with schizophrenia.

Other treatable diseases and disorders include dysthymia and cyclothymia.

Other treatable diseases and disorders include delirium, dementia, and amnestic and other cognitive or neurodegenerative disorders, such as Parkinson's disease, Huntington's disease, Alzheimer's disease, senile dementia, dementia of the Alzheimer's type, memory disorders, loss of executive function, vascular dementia, and other dementias, for example, due to human immunodeficiency virus (HIV) disease, head trauma, Parkinson's disease, Huntington's disease, Pick's disease, Creutzfeldt-Jakob disease, or due to multiple etiologies.

Other treatable diseases and disorders include movement disorders such as akinesias, dyskinesias, including familial paroxysmal dyskinesia, spasticities, Tourette's syndrome, Scott syndrome, palsys (e.g., Bell's palsy, cerebral palsy, birth palsy, brachial palsy, wasting palsy, ischemic palsy, progressive bulbar palsy and other palsys), and akinetic-rigid syndrome. Other treatable diseases and disorders include extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor.

Other treatable diseases and disorders include chemical dependencies and addictions (e.g., dependencies on, or addictions to, alcohol, heroin, cocaine, benzodiazepines, nicotine, or phenobarbitol) and behavioral addictions such as an addiction to gambling.

Other treatable diseases and disorders include ocular disorders such as glaucoma and ischemic retinopathy.

Other treatable diseases and disorders include autism and pervasive development disorder.

Another treatable disease or disorder is pain. Pain refers to acute as well as chronic pain. Acute pain is usually short-lived and is associated with hyperactivity of the sympathetic nervous system. Examples of acute pain are postoperative pain and allodynia. Chronic pain may be defined as pain persisting for more than 3 months and includes somatogenic pain and psychogenic pain. Other examples of treatable pain include nociceptive pain and neuropathic pain.

Other examples of treatable pain include pain resulting from soft tissue or peripheral damage such as acute trauma. Another example is musculo-skeletal pain such as pain experienced after trauma.

Other examples of treatable pain include pain associated with arthritis including pain associated with osteoarthritis or rheumatoid arthritis, including non-neuropathic arthritic pain and neuropathic arthritic pain. Other examples include pain resulting from ankylosing spondylitis or gout.

Other examples of treatable pain include pain associated with fibromyalgia, including non-neuropathic fibromyalgic pain and neuropathic fibromyalgic pain.

Other examples of treatable pain include chronic non-neuropathic pain such as pain associated with: HIV, arthralgia, myalgia, sprains, strains, or trauma such as broken bones, and chronic post surgical pain.

Other examples of treatable pain include spinal pain, dental pain, myofascial pain syndromes, episiotomy pain, and pain resulting from a burn.

Other examples of treatable pain include deep and visceral pain, such as heart pain, muscle pain, eye pain, orofacial pain, for example, odontalgia, abdominal pain, gynecological pain, for example, dysmenorrhoea, labor pain, and pain associated with endometriosis.

Other examples of treatable pain include pain associated with nerve and root damage (e.g., neuropathic pain) such as pain associated with a peripheral nerve disorder, for example, nerve entrapment and brachial plexus avulsion, amputation, a peripheral neuropathy, tic douloureux, atypical facial pain, nerve root damage, trigeminal neuralgia, neuropathic lower back pain, HIV related neuropathic pain, cancer related neuropathic pain, diabetic neuropathic pain, and arachnoiditis.

Other examples of treatable pain include neuropathic and non-neuropathic pain associated with carcinoma, often referred to as cancer pain, central nervous system pain such as pain due to spinal cord or brain stem damage, lower back pain, sciatica, and phantom limb pain. Other examples include headache, including migraine and other vascular headaches, acute or chronic tension headache, cluster headache, temperomandibular pain, and maxillary sinus pain. Other examples of treatable pain are pain caused by increased bladder contractions and scar pain.

Other examples of treatable pain include pain that is caused by injury or infection of peripheral sensory nerves. Examples include neuropathic pain and pain from: peripheral nerve trauma, herpes virus infection, diabetes mellitus, fibromyalgia, causalgia, plexus avulsion, neuroma, limb amputation, or vasculitis. Neuropathic pain is also caused by nerve damage from chronic alcoholism, HIV infection, hypothyroidism, uremia, or vitamin deficiencies. Neuropathic pain includes, but is not limited to pain caused by nerve injury such as, for example, diabetic neuropathy.

Another example of treatable pain is psychogenic pain, which occurs without an organic origin, and includes low back pain, atypical facial pain, and chronic headache.

Other examples of treatable pain are inflammatory pain, pain associated with restless legs syndrome, acute herpetic neuralgia, postherpetic neuralgia, occipital neuralgia, and other forms of neuralgia, neuropathic pain syndrome, and idiopathic pain syndrome.

In some embodiments, pain associated with fibromyalgia is being treated. In some embodiments, pain associated with osteoarthritis is being treated. In other embodiments, pain associated with rheumatoid arthritis is being treated.

In some embodiments, attention deficit hyperactivity disorder is being treated. In other embodiments, neuropathic pain is being treated. In other embodiments, anxiety is being treated. In other embodiments, depression is being treated. In other embodiments, schizophrenia is being treated.

Another embodiment is a combination comprising a compound of Formula (I), or a pharmaceutically acceptable acid addition salt thereof, and behavior modification therapy. Examples of behavior modification therapy that may be used in the combination are behavior modification therapy for the treatment of depression, anxiety, a phobia, or ADHD.

In some embodiments, a compound of Formula (I), or a pharmaceutically acceptable acid addition salt thereof, is simultaneously or sequentially “co-administered” with another pharmaceutically active compound (e.g., a compound useful for treating the above-named diseases and disorders), or a pharmaceutically acceptable acid addition salt thereof. Simultaneously co-administering includes administering a pharmaceutical co-composition comprising: (i) a compound of Formula (I), or a pharmaceutically acceptable acid addition salt thereof, (ii) a pharmaceutically active ingredient that is not a compound of Formula (I), or a pharmaceutically acceptable salt of the ingredient, and (iii) a pharmaceutically acceptable excipient. Components (i) and (ii) may or may not be in direct physical contact with each other in the co-composition and may be formulated with the same or different excipient(s). Simultaneously administering also includes administering two or more separate pharmaceutical compositions at about the same time such as starting each co-administration within about 1 hour of each other. Sequentially co-administering includes sequentially administering (i.e., at different times such as starting the co-administrations more than 1 hour apart) two or more separate pharmaceutical compositions. In some embodiments, the co-administering is simultaneous and the active ingredients are found together in a pharmaceutical co-composition.

Examples of pharmaceutically active compounds that are not compounds of Formula (I) include NSAIDs such as piroxicam; loxoprofen; diclofenac; propionic acids such as naproxen, flurbiprofen, fenoprofen, ketoprofen and ibuprofen; ketorolac; nimesulide; acetominophen; fenamates such as mefenamic acid; indomethacin; sulindac; apazone; pyrazolones such as phenylbutazone; salicylates such as aspirin; cyclooxygenase-2 (COX-2) inhibitors such as celecoxib, valdecoxib, parecoxib, and etoricoxib; steroids; cortisone; prednisone; muscle relaxants including cyclobenzaprine and tizanidine; hydrocodone; dextropropoxyphene; lidocaine; opioids such as morphine, fentanyl, tramadol, and codeine; paroxetine; diazepam; femoxetine; carbamazepine; milnacipran; reboxetine; venlafaxine; duloxetine; topisetron; interferon alpha; cyclobenzaprine; CPE-215; sodium oxbate; citalopram HBr; sertraline HCl; antidepressants, tricyclic antidepressants, amitryptyline, fluoxetine; topiramate; escitalopram; benzodiazepenes including diazepam, bromazepam and tetrazepam; mianserin; clomipramine; imipramine; topiramate; and nortriptyline. Other examples include alpha-2-delta (A2D) ligands such as those compounds generally or specifically disclosed in U.S. Pat. No. 4,024,175, particularly gabapentin; U.S. Pat. No. 6,197,819, particularly pregabalin; U.S. Pat. Nos. 5,563,175; 6,020,370; 6,103,932; and 5,929,088; U.S. Pat. No. 6,596,900, particularly [(1R,5R,6S)-6-(aminomethyl)bicyclo[3.2.0]hept-6-yl]acetic acid; U.S. Pat. No. 6,518,289, U.S. Pat. No. 6,545,022, and U.S. Pat. No. 6,521,650, particularly 3-(1-aminomethyl-cyclohexylmethyl)-4H-[1,2,4]oxadiazol-5-one and C-[1-(1H-tetrazol-5-ylmethyl)-cycloheptyl]-methylamine; U.S. Pat. No. 6,635,673 and U.S. Pat. No. 6,921,835, particularly (3S,4S)-(1-aminomethyl-3,4-dimethyl-cyclopentyl)-acetic acid; U.S. Patent Application Publication No. US2005-059735; U.S. Pat. No. 6,689,906 and U.S. Pat. No. 6,835,751, particularly (1α,3α,5α)(3-amino-methyl-bicyclo[3.2.0]hept-3-yl)-acetic acid; U.S. Pat. No. 6,153,650; U.S. Pat. No. 6,642,398, particularly (3S,5R)-3-aminomethyl-5-methyl-octanoic acid; U.S. Patent Application Publication No. US2005-272783, particularly (3S,5R)-3-amino-5-methyl-heptanoic acid, (3S,5R)-3-amino-5-methyl-nonanoic acid, and (3S,5R)-3-Amino-5-methyl-octanoic acid; U.S. Pat. Nos. 6,703,522; U.S. Pat. No. 6,846,843; U.S. Pat. No. 6,818,787, U.S. Pat. No. 6,833,140, U.S. Pat. No. 6,972,341, U.S. Pat. No. 6,824,228, and U.S. Patent Application Publication Nos. US2003-203945, US2004-171682, US2003-229145, and US2003-225084, and pharmaceutically acceptable acid addition salts and solvates thereof.

For the treatment of depression or anxiety, the compounds of the invention can be used in combination with one or more other antidepressants or anti-anxiety agents. Examples of classes of the antidepressants that can be used include norepinephrine reuptake inhibitors (NRIs), selective serotonin reuptake inhibitors (SSRIs), norepinephrine and serotonin reuptake inhibitors (NSRIs), serotonin and norepinephrine reuptake inhibitors (SNRIs), neurokinin-1 (NK-1) receptor antagonists, monoamine oxidase inhibitors (MAOIs), reversible inhibitors of monoamine oxidase (RIMAs), corticotropin releasing factor (CRF) antagonists, α-adrenoreceptor antagonists, A2D ligands, and atypical antidepressants. Suitable norepinephrine reuptake inhibitors include tertiary amine tricyclics and secondary amine tricyclics (e.g., tricyclic antidepressants). Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine, citalopram, and sertraline. Examples of monoamine oxidase inhibitors include isocarboxazid, phenelzine, and tranylcyclopramine. Suitable reversible inhibitors of monoamine oxidase include moclobemide. Suitable serotonin and noradrenaline reuptake inhibitors include venlafaxine and duloxetine. Suitable CRF antagonists include those compounds described in U.S. Pat. No. 6,448,265; U.S. Pat. Nos. 5,668,145; 5,705,646; U.S. Pat. No. 6,765,008; and U.S. Pat. No. 6,218,397. Suitable atypical anti-depressants include bupropion, lithium, nefazodone, trazodone and viloxazine. Suitable NK-1 receptor antagonists include those referred to in U.S. Patent Application Publication No. US2003-087925. Suitable A2D ligands include those referenced above, including gabapentin and pregabalin.

Suitable classes of anti-anxiety agents that can be used in combination with the active compounds of the invention include benzodiazepines, CRF antagonists, and serotonin-1A (i.e., 5-hydroxytryptamine-1A (5-HT_{1A})) agonists or antagonists, especially 5-HT_{1A} partial agonists. Suitable benzodiazepines include alprazolam, chlordiazepoxide, clonazepam, chlorazepate, diazepam, halazepam, lorazepam, oxazepam, and prazepam. Suitable 5-HT_{1A} receptor agonists or antagonists include buspirone, flesinoxan, gepirone and ipsapirone.

For the treatment of schizophrenia, the compounds of the invention can be used in combination with one or more other antipsychotic agent. Suitable antipsychotic agents include both conventional and atypical antipsychotics. Conventional antipsychotics are antagonists of another monoamine neurotransmitter dopamine, especially dopamine-2 (D₂) receptors. The atypical antipsychotics also have D₂ antagonistic properties but possess different binding kinetics to these receptors and activity at other receptors, particularly 5-HT_{2A}, 5-HT_{2C} and 5-HT_{2D}. The class of atypical antipsychotics includes clozapine, 8-chloro-11-(4-methyl-1-piperazinyl)-5H-dibenzo[b,e][1,4]diazepine (U.S. Pat. No. 3,539,573); risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one (U.S. Pat. No. 4,804,663); olanzapine, 2-methyl-4-(4-methyl-1-piperazinyl)-10H-thieno[2,3-b][1,5]benzodiazepine (U.S. Pat. No. 5,229,382); quetiapine, 5-[2-(4-dibenzo[b,f][1,4]thiazepin-11-yl-1-piperazinyl)ethoxy]ethanol (U.S. Pat. No. 4,879,288); aripiprazole, 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro-2(1H)-quinolinone (U.S. Pat. Nos. 4,734,416 and 5,006,528); sertindole, 1-[2-[4-[5-chloro-1-(4-fluorophenyl)-1H-indol-3-yl]-1-piperidinyl]ethyl]imidazolidin-2-one (U.S. Pat. No. 4,710,500); amisulpride (U.S. Pat. No. 4,410,822); and ziprasidone, 5-[2-[4-(1,2-benzisothiazol-3-yl)piperazin-3-yl]ethyl]-6-chloroindolin-2-one hydrochloride hydrate (U.S. Pat. No. 4,831,031).

Compounds of Formula (I), and intermediates and starting materials in the syntheses thereof, may be prepared by one of ordinary skill in the art using conventional synthetic chemistry methods. Some starting materials may also be obtained from a commercial supplier such as the Sigma-Aldrich Company, St. Louis, Mo.

Syntheses of some of the compounds of Formula (I) may utilize starting materials, intermediates, or reaction products that contain more than one reactive functional group. During chemical reactions, a reactive functional group may be protected from unwanted side reactions by a protecting group that renders the reactive functional group substantially inert to the reaction conditions employed. A protecting group is selectively introduced onto a starting material prior to carrying out the reaction step for which a protecting group is needed. Once the protecting group is no longer needed, the protecting group can be removed. It is well within the ordinary skill in the art to introduce protecting groups during a synthesis of a compound of formula (I) and then later remove them. Procedures for introducing and removing protecting groups are known, for example, in Protective Groups in Organic Synthesis, 3^{rd} ed., Greene T. W. and Wuts P. G., Wiley-Interscience, New York, 1999.

The following moieties are examples of protecting groups that may be utilized to protect amino, hydroxyl, or other functional groups: carboxylic acyl groups such as, for example, formyl, acetyl, and trifluoroacetyl; alkoxycarbonyl groups such as, for example, ethoxycarbonyl, tert-butoxycarbonyl (BOC), β,β,β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removing protecting groups include hydrogenolysis of CBZ groups using, for example, hydrogen gas at about 3.4 atmospheres in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Illustrative syntheses of compounds of Formula (I) are outlined in Schemes (A), (B), (C), (D), and (E).

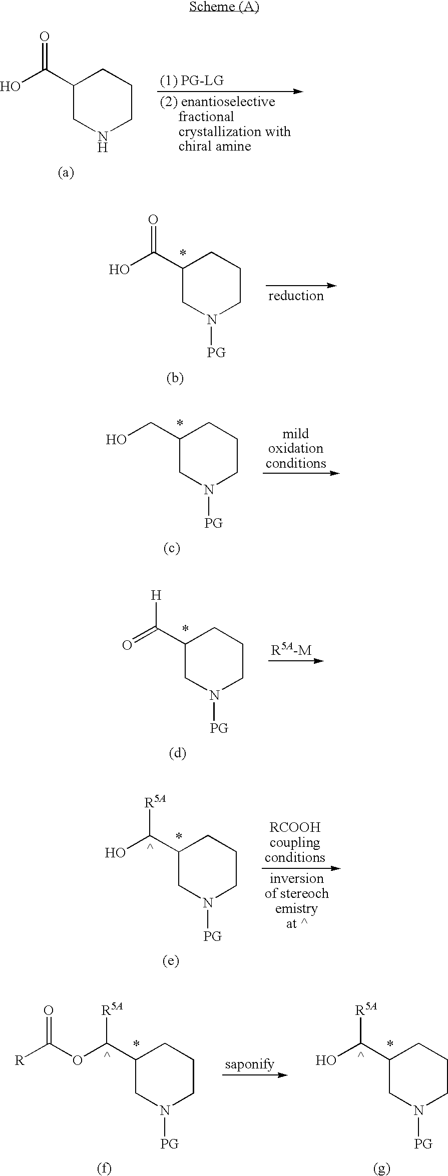

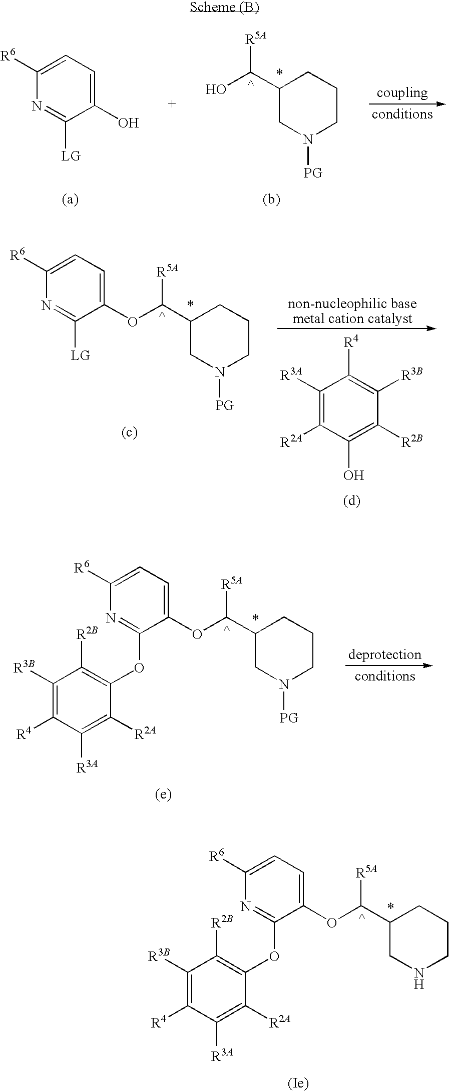

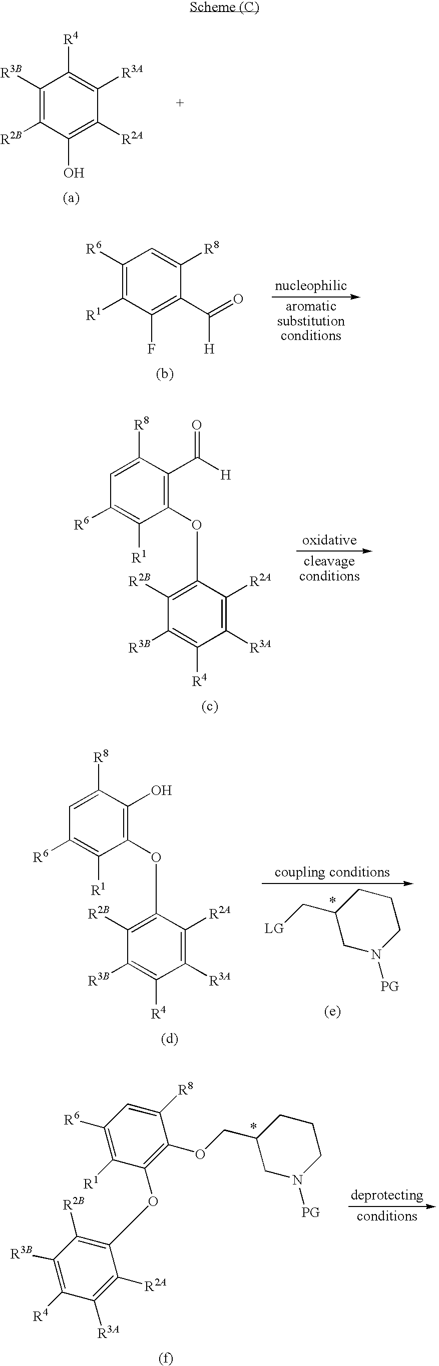

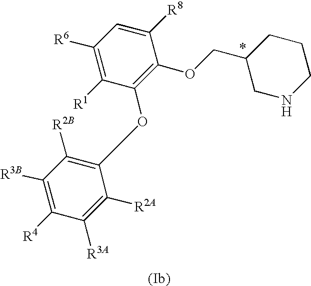

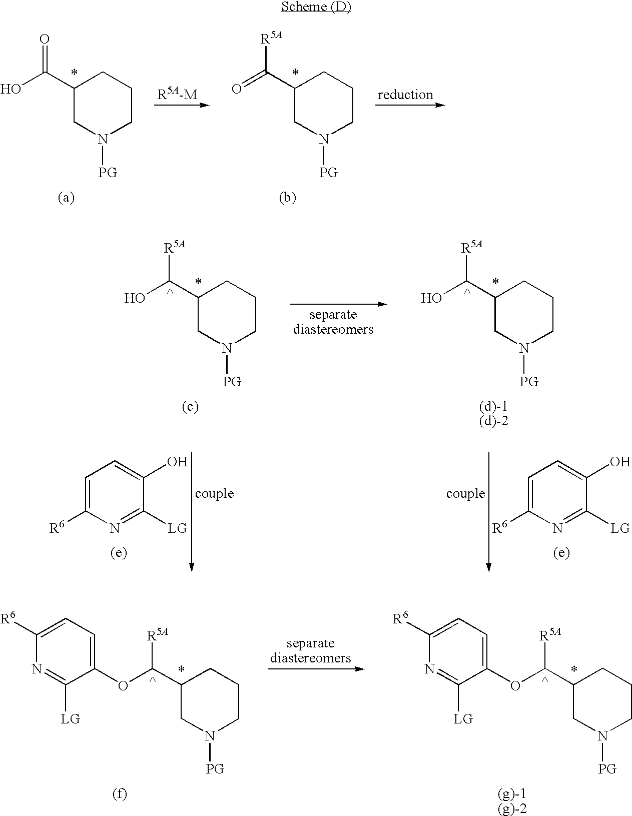

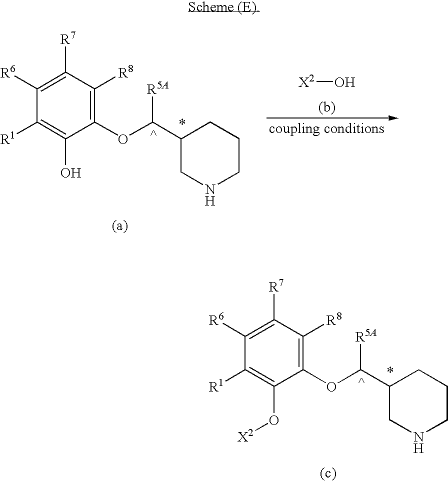

In Scheme (A), the nitrogen of (±)-nipecotic acid (a) (Aldrich Chemical Company Catalog No. 211672) is protected using conventional amino acid protecting group chemistry such as that described in Protective Groups in Organic Synthesis (supra) with protecting group PG, wherein PG is an amine protecting group such BOC or CBZ to give N-protected-(±)-nipecotic acid. The individual enantiomers of N-protected-(±)-nipecotic acid are separated using conventional enantioselective fractional crystallization with a chiral amine or conventional enantioselective chromatography of a chiral ester derivative of the N-protected-(±)-nipecotic acid to give (S)— or (R)—N-protected-nipecotic acid (b). Examples of suitable chiral amines are 1-tert-leucinol, (+)-cinchonine, L-proline, L-phenyl glycine methyl ester, L-valinol, (1R,2R)-(−)-1,2-diaminocyclohexane, (S)-(−)-α-methyl-benzylamine, (1R,2S)-(−)-ephedrine, L-phenylalaninol, (1S,2R)-(+)-norephedrine, (R)-(+)-N-benzyl-α-methylbenzylamine, (−)-cinchonidine, (+)-cinchonine, and (−)-quinine.

The (S)— or (R)—N-protected-nipecotic acid (b) is reduced using a suitable hydride reducing conditions such as borane in tetrahydrofuran (THF), lithium aluminum hydride in THF, and the like at a temperature from −20° C. to 50° C. to give an (S)— or (R)—N-protected-piperidin-3-ylmethanol (c).

The (S)— or (R)—N-protected-piperidin-3-ylmethanol (c), which is also used as illustrated in Schemes (B) and (C), oxidized to the corresponding aldehyde (d) using an oxidant such as 2-iodoxybenzoic acid or dimethylsulfoxide (DMSO)/oxalyl chloride/trimethyl amine in an aprotic solvent such as dichloromethane, THF, or ethyl acetate at a temperature from −20° C. to 100° C.

The aldehyde (d) is allowed to react with an organometallic agent R^{5A}-M, wherein R^{5A} is as defined herein, preferably (C₁-C₄)alkyl, and M is Li⁺, ½ Zn⁺², or ½ Mg⁺² cation, preferably ½ Zn⁺², in the presence of a chiral auxiliary such as (1R)-trans-N,N′-1,2-cyclohexanediylbis(1,1,1-trifluoromethanesulfonamide) and an optional Lewis acid such as titanium isopropoxide in an aprotic solvent such as ethyl ether, THF, and the like at a temperature from −50° C. to room temperature to give the secondary alcohol (e). For example, when N-BOC-(S)-aldehyde (d) is allowed to react with diethyl zinc in the presence of (1R)-trans-N,N′-1,2-cyclohexanediylbis(1,1,1-trifluoromethanesulfonamide) and titanium isopropoxide in ethyl ether, (R)-1-[(S)—N-BOC-piperidin-3-yl]-propanol is obtained.

The stereochemistry at a second chiral carbon, which is indicated with the symbol ̂, in secondary alcohol (e) can be inverted by allowing the compound to couple with a carboxylic acid such as benzoic acid under conditions that lead to inversion such as using triphenylphosphine, diisopropylazodicarboxylate (DIAD), in 1,2-dimethoxyethane (DME) at a temperature from 0° C. to 100° C., preferably from room temperature to 65° C., to give the ester (f), which can then be saponified using conventional conditions such as sodium hydroxide in THF or methanol and optionally water at a temperature from 0° C. to about reflux to give the secondary alcohol (g), wherein the stereochemistry at a second chiral carbon in secondary alcohol (g) is epimeric to the stereochemistry at the second chiral carbon in secondary alcohol (e). The secondary alcohols (e) and (g) can be used in the synthesis of a compound of the invention or salt thereof as illustrated in Schemes (B), (C), and (D).

In Scheme (B), a 2-substituted-pyridin-3-ol (a), wherein LG is a leaving group such as bromo or iodo, is allowed to react with an N-protected-piperidin-3-ylmethanol (b), wherein PG is BOC or CBZ, and the N-protected-piperidin-3-ylmethanol (b) is prepared as described for Scheme (A), under suitable coupling conditions to give the ether (c). Examples of suitable coupling conditions are an aprotic solvent such as THF, dioxane, or 1,2-dimethoxyethane at a temperature from about 5° C. to about 100° C., preferably from room temperature to 65° C., in the presence of a coupling agent useful for coupling an acidic —OH with an alcoholic —OH. Such coupling agents include triphenylphosphine with DIAD; 1-(3-dimethylaminopropyl)-3-ethylcarbodiimide hydrochloride (EDC, EDCI, or EDAC), N,N′-carbonyldiimidazole (CDI), or N,N′-dicyclohexylcarbodiimide (DCC), each optionally with 1-hydroxybenzotriazole (HOBt); or (benzotriazol-1-yloxy)tripyrrolidino-phosphonium hexafluorophosphate.

Alternatively, the N-protected-piperidin-3-ylmethanol (b) is allowed to react with a suitable sulfonyl chloride such as methanesulfonyl or tosyl chloride in the presence of a suitable non-nucleophilic base such as excess potassium carbonate or excess sodium hydride in an aprotic polar solvent such as acetonitrile or tetrahydrofuran (THF) at a temperature from about 5° C. to about 100° C., preferably from room temperature to 80° C., to form the corresponding sulfonate in situ, which is then allowed to react with the 2-substituted-pyridin-3-ol (a) to give the ether (c).

The ether (c) is then coupled with the phenol (d) under suitable conditions to give the bis-ether (e). Examples of suitable conditions are an aprotic solvent such as THF, dioxane, or 1,2-dimethoxyethane at a temperature from about 25° C. to about 150° C. in the presence of a non-nucleophilic base such as potassium tert-butoxide (KTBU), potassium hydride (KH), potassium hexamethyldisilazide (KHMDS), or the like and a coupling catalyst useful for catalyzing a coupling of an aromatic bromide or iodide with a phenol. These coupling catalysts include copper(I) triflate and copper(I) iodide, which may be generated in situ with copper(I) triflate-benzene complex or copper(I) triflate-toluene complex and the aromatic bromide or iodide.

The bis-ether (e) is then deprotected under suitable conditions to give a compound of Formula (Ia), which is a compound of Formula (I) wherein X¹ is N. Examples of suitable deprotecting conditions are a strong acid such as hydrogen chloride or trifluoroacetic acid in an aprotic solvent such as dichloromethane or acetonitrile at a temperature from about 5° C. to about 50° C., preferably about room temperature.

In Scheme (C), a phenol (a) is allowed to react with a 2-fluorobenzaldehyde (b) under suitable coupling conditions to give an aldehyde (c). Examples of suitable coupling conditions are an aprotic polar solvent such as N,N-dimethylacetamide (DMA), N,N-dimethylformamide (DMF), dimethylsulfoxide (DMSO), and the like at a temperature from about 5° C. to about 100° C. in the presence of a non-nucleophilic base such as cesium carbonate, sodium hydride, and the like.

The aldehyde (c) is then oxidatively cleaved under suitable conditions to give the phenol (d). Examples of suitable cleavage conditions are an aprotic solvent such as dichloromethane, chloroform, chlorobenzene, and the like and a mild acid such as KH₂PO₄, KHSO₄, and the like, at a temperature from about 25° C. to about 100° C. in the presence of a peroxide such as 3-chloro-peroxybenzoic acid.

The phenol (d) is then allowed to react under suitable coupling conditions with an N-protected-piperidin-3-ylmethanol derivative (e), wherein PG is an amine protecting group such BOC or CBZ and LG is a leaving group such as a methanesulfonate, trifluoromethanesulfonate, tosylate, bromide, and the like, to give the bis-ether (f). Examples of suitable coupling conditions are an aprotic polar solvent such as THF, acetonitrile, DMA, and the like at a temperature from about 5° C. to about 100° C. in the presence of a non-nucleophilic base such as cesium carbonate, sodium carbonate, sodium hydride, and the like.

The bis-ether (f) is then deprotected under suitable conditions to give a compound of Formula (Ib), which is a compound of Formula (I) wherein X¹ is C—R¹. Examples of suitable deprotecting conditions are a strong acid such as hydrogen chloride or trifluoroacetic acid in an aprotic solvent such as dichloromethane or acetonitrile.

In Scheme (D), (S)— or (R)—N-protected-nipecotic acid (a), which corresponds to compound (b) in Scheme (A), is allowed to react with an activating agent such as ethyl chloroformate, thionyl chloride, or oxalyl chloride, and then coupled with N,O-dimethylhydroxylamine hydrochloride in the presence of a tertiary amine base such as N-methyl-piperidine in an aprotic solvent such as dichloromethane, acetonitrile, or ethyl ether at a temperature from −78° C. to room temperature to give the corresponding N,O-dimethyl-(S)— or (R)—N-protected-nipecotic amide, which is isolated and then allowed to react with an organometallic agent R^{5A}-M, wherein R^{5A} is as defined herein, preferably phenyl, and M is Li⁺, ½ Zn⁺², or ½ Mg⁺² cation, preferably ½ Mg⁺², in a suitable solvent such as THF, ethyl ether, or DME at a temperature from −20° C. to room temperature, preferably 0° C., to give the ketone (b).

The ketone (b) is reduced with a hydride reducing agent such as sodium borohydride or lithium aluminum hydride in a solvent such as THF, methanol, or ethanol at a temperature from −20° C. to 50° C., preferably room temperature, to give a mixture of diastereomers of alcohol (c) that is a mixture of epimers at a second chiral carbon, which is indicated by the symbol ̂. Alternatively, a chiral hydride reducing agent could be used, which would provide predominantly one of the two possible epimers at the second chiral carbon in alcohol (c).

The mixture of two diastereomers of alcohol (c), wherein the stereochemistry at the first chiral carbon was predetermined according to whether (S)— or (R)—N-protected-nipecotic acid (a) was used, optionally may be separated by chromatography such as chromatography on silica gel by eluting with a single solvent or a mixture of solvents to independently give isolated epimeric alcohols (d)-1 and (d)-2. Each isolated epimeric alcohol (d)-1 and (d)-2 independently can be coupled with a pyrindin-3-ol (e), wherein LG is a leaving group such as bromo or iodo and R⁶ is as defined herein, under coupling conditions such as those described herein for coupling a phenol or pyridinol with an alcohol (e.g., triphenylphosphine and DIAD in toluene or DME) at a temperature from 0° C. to 100° C., preferably from room temperature to 65° C., to independently give epimeric ethers (g)-1 and (g)-2, respectively.

Alternatively, the mixture of two diastereomers of alcohol (c) optionally can be coupled with the pyrindin-3-ol (e) under the coupling conditions such as those described herein for coupling a phenol or pyridinol with an alcohol to give a mixture of diastereomers of ether (f), that is a mixture of epimers at a second chiral carbon, which is indicated by the symbol ̂. The mixture of diastereomers of ether (f) may be separated by chromatography such as chromatography on silica gel by eluting with a single solvent or a mixture of solvents to independently give the isolated epimeric ethers (g)-1 and (g)-2.

Not shown in Scheme (D), each epimeric ether (g)-1 and (g)-2 may be coupled with the phenol (d) of Scheme (B) using the conditions outlined above for Scheme (B) to give a compound of Formula (Ic).

Alternatively, each epimeric ether (g)-1 and (g)-2 may be coupled with an alcohol of formula (A)

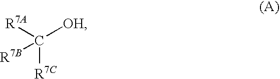

wherein R^{7A}, R^{7B}, and R^{7C} are as defined herein, using a non-nucleophilic base such as sodium hydride optionally in the presence of a coupling catalyst useful for catalyzing a coupling of an aromatic bromide or iodide with an alcohol at a temperature from room temperature to about 150° C., preferably about 100° C., in an aprotic solvent such as DME or toluene to give a compound of Formula (Ie). These coupling catalysts include copper(I) triflate and copper(I) iodide, which may be generated in situ with copper(I) triflate-benzene complex or copper(I) triflate-toluene complex and the epimeric ether (g)-1 or (g)-2.

Alternatively, the secondary alcohols (e) or (g) of Scheme (A) or epimeric alcohols (d)-1 or (d)-2 of Scheme (D) may be coupled with phenol (d) of Scheme (C) using the conditions outlined above for Scheme (C) to give a compound of Formula (Id).

In Scheme (E), an alcohol of formula (b) is coupled with a phenol of formula (a) using conventional coupling conditions such as triphenylphosphine and diisopropyldiazodicarboxylate or some other coupling reagent such as dicyclohexyldicarboxylate to in an aprotic polar solvent at a temperature of from 0° C. to about 100° C. to give the ether of formula (c).

The compounds of Formula (I) may be synthesized in racemic form or in a chiral form, which means any non-racemic mixture. Racemic mixtures are typically prepared from racemic starting materials. Chiral forms may be prepared from chiral starting materials. Alternatively, chiral forms may be prepared from their respective racemic forms using conventional enantioselective separation methods, which separate the chiral components of the racemic forms of the compounds of Formula (I), or the racemic intermediates in the synthesis thereof.

Examples of conventional enantioselective separation methods are enantioselective fractional crystallization and enantioselective chromatography, including enantioselective multi-column chromatography. Generally illustrative pharmaceutical industry applications of enantioselective multi-column chromatography are described in U.S. Pat. Nos. 5,928,515; 5,939,552; 6,107,492; 6,130,353; 6,455,736; and 6,458,955. Enantioselective fractional crystallization of the racemic forms of the compounds of Formula (I) may be accomplished by crystallizing salts with chiral carboxylic acids such as L-(+)-tartaric acid or chiral sulfonic acids such as either (1R)-(−)-10-camphorsulfonic acid or (1S)-(+)-10-camphorsulfonic acid, and then converting the salts of the separated stereoisomers of the compounds of Formula (I) back to their free base forms in a conventional manner.

Syntheses of the compounds of Formula (I) may use chiral intermediates such as (S)- and (R)-3-hydroxymethyl-piperidine-1-carboxylic acid tert-butyl esters. The (S)- and (R)-3-hydroxymethyl-piperidine-1-carboxylic acid tert-butyl esters may be prepared from the corresponding (S)- or (R)-nipecotic acid ethyl esters using conventional methods. (S)- and (R)-nipecotic acid ethyl esters are each commercially available from commercially available from ABCR GmbH & Co. KG, Im Schlehert 10, D-76187 Karlsruhe, Germany (ABCR). The esters have been assigned Chemical Abstracts Service Registry Numbers (CAS Reg. Nos.) [37675-18-6] and [25137-01-3], respectively. Also, (S)—N-t-butyloxycarbonyl-nipecotic acid is commercially available from ABCR under Product Number AB156118/BAA1203. The (S)- and (R)-nipecotic acids are also commercially available from ABCR and from Yamakawa Chemical Industry Co., Limited, Tanaka Building, 3-1-10, Nihonbashi-Muromachi, Chuo-ku Tokyo 103-0022, Japan. The acids have been assigned CAS Reg. Nos. [59045-82-8] and [25137-00-2], respectively.

### Preparation 1

### Synthesis of (S)-3-(2-iodo-6-methyl-pyridin-3-yloxymethyl)-piperidine-1-carboxylic acid tert-butyl ester

(S)-3-Hydroxymethyl-piperidine-1-carboxylic acid tert-butyl ester (3.27 g, 15.2 mmol), which may be prepared using conventional methods from (±)-nipecotic acid (Aldrich Chemical Company Catalog No. 211672), 2-iodo-6-methyl-pyridin-3-ol (4.03 g, 17.2 mmol), and triphenylphosphine (4.8 g, 18 mmol) were charged to a 100 mL flask. Then 1,2-dimethoxyethane (15 mL) was added, followed by diisopropylazodicarboxylate (3.7 g, 18 mmol). The resulting solution was stirred at 50° C. for 5 hours. After rotary evaporation in vacuo, the residue was chromatographed on silica gel, eluting with a linear gradient 0-65% of (11 parts ethyl acetate and 60 parts dichloromethane) and 100-35% dichloromethane. The residue was dissolved in ethyl ether (120 mL) and washed 2 times with 15% aqueous sodium hydroxide (10-15 mL), dried over MgSO₄, and rotary evaporated in vacuo to give the title compound as an oil (6.15 g), which solidified on standing.

### Preparation 2

### Synthesis of (S)-3-[2-(4-fluoro-2-methyl-phenoxy)-6-methyl-pyridin-3-yloxymethyl]-piperidine-1-carboxylic acid tert-butyl ester

(S)-3-(2-Iodo-6-methyl-pyridin-3-yloxymethyl)-piperidine-1-carboxylic acid tert-butyl ester (0.349 g, 0.807 mmol) from Preparation 1,4-fluoro-2-methyl-phenol (0.15 g, 1.2 mmol), and 1,2-dimethoxyethane (2.5 mL) were charged to an 8 mL septum-capped vial. The resulting mixture was stirred, and potassium tert-butoxide (0.14 g, 1.2 mmol) was added, followed by about 10 mg of copper (I) triflate benzene complex. The vial was placed in a dry block heated at 100° C. on a stirrer/hotplate for 18-24 hours. The reaction mixture was chromatographed on silica gel, eluting with a linear gradient 0-45% ethyl acetate and 100-55% hexanes to give the title compound as a yellow oil (272 mg).

### Preparation 3

### Synthesis of 4-Chloro-2-(2-fluoro-6-methoxy-phenoxy)-benzaldehyde

A stirred solution of 4-chloro-2-fluoro-benzaldehyde (3.58 g, 25.23 mmol) and 2-fluoro-6-methoxy phenol (4.0 g, 25 mmol) in DMA (25 mL) was treated with cesium carbonate (8.22 g, 25.23 mmol). The mixture was stirred at room temperature for a total of 48 hours. The reaction mixture was poured into about 150 mL of ice water and stirred for 6 hours. The resulting solid was removed by filtration, washed with water and dried at 45° C. in a vacuum oven for 18 hours to give 6.8 g (97%) of the title compound.

### Preparation 4

### Synthesis of 4-chloro-2-(2-fluoro-6-methoxy-phenoxy)-phenol

A solution of 4-chloro-2-(2-fluoro-6-methoxy-phenoxy)-benzaldehyde (6.8 g, 24 mmol) from Preparation 3 in CHCl₃ (100 mL) was treated with solid KH₂PO₄ (4.9 g, 36 mmol) followed by solid technical grade (57-86% pure) 3-chloroperoxybenzoic acid (6.3 g, 36 mmol). The mixture was stirred at 55° C. for 20 hours. The solution was treated with additional 3-chloroperoxybenzoic acid (1.5 g, 8.6 mmol), solid KH₂PO₄ (1.0 g, 7.3 mmol) and the stirring continued for an additional 6 hours. The mixture was cooled to room temperature and extracted with saturated aqueous NaHCO₃, brine, and dried over MgSO₄. The mixture was filtered and rotary evaporated under reduced pressure. The residue was dissolved. In 150 mL methanol, treated with 3 drops of concentrated HCl, and heated to reflux for 18 hours. The cooled solution was rotary evaporated under reduced pressure. The residue was crystallized from ethyl ether-hexane to afford 1.6 g 25%) of the title compound.

### Preparation 5

### Synthesis of (S)-2-[4-chloro-2-(2-fluoro-6-methoxy-phenoxy)-phenoxymethyl]piperidine-1-carboxylic acid tert-butyl ester

A mixture of 4-chloro-2-(2-fluoro-6-methoxy-phenoxy)-phenol (0.30 g, 1.0 mmol), from Preparation 4, (S)-3-methanesulfonylmethyl-piperidine-1-carboxylic acid tert-butyl ester (0.41 g, 1.5 mmol) (which was prepared according to the procedure of Preparation 22), and solid cesium carbonate (0.60 g, 1.8 mmol) in 5 mL of acetonitrile (5 mL) can be heated to reflux with stirring for a total of 48 hours. The reaction can be cooled to room temperature and the solvent removed under reduced pressure. The residue can be dissolved in ethyl acetate, extracted with 1 N NaOH, brine, and dried over MgSO₄. The mixture can be filtered and rotary evaporated under reduced pressure. The residue can be purified on a silica gel column using a hexane/ethyl acetate mobile phase. The appropriate fractions can be combined and the solvent removed under pressure to give the title compound.

The piperidine nitrogen of (S)-2-[4-chloro-2-(2-fluoro-6-methoxy-phenoxy)-phenoxymethyl]piperidine-1-carboxylic acid tert-butyl ester can be deprotected by one of ordinary skill in the art adapting the procedure of Example 1.

### Preparation 6

### Synthesis of (S)-3-(2-bromo-pyridin-3-yloxymethyl)-piperidine-1-carboxylic acid tert-butyl ester

To a stirring mixture at room temperature of (S)-3-hydroxymethyl-piperidine-1-carboxylic acid, tert-butyl ester (7.32 g, 34.0 mmol), 2-bromo-3-pyridinol (7.40 g, 42.5 mmol) and triphenylphosphine (11.15 g, 42.5 mmol) in 35 mL of toluene was added dropwise diisopropyl azodicarboxylate (8.4 mL, 42.7 mmol). Addition was exothermic, after which all solid was in solution. The solution was heated at 65° C. under N₂ for 24 hours, rotary evaporated to remove most of the toluene, and then suspended into 200 mL of a (about 1:1) hexanes:diethyl ether mixture. The solid that formed was removed by filtration. The filtrate was rotary evaporated, and the residue was redissolved into diethyl ether, then washed 2 times with 1 N NaOH, then with saturated aqueous KH₂PO₄, and brine solutions. The organic extract was dried (MgSO₄), filtered, and rotary evaporated to give a residue, which was chromatographed (medium pressure liquid chromatography or MPLC, silica gel, 5% EtOAc in CH₂Cl₂) to give 9.36 g (74%) of (S)-3-(2-bromo-pyridin-3-yloxymethyl)-piperidine-1-carboxylic acid tert-butyl ester as an off-white solid, mp 79-81° C. Elemental Analysis: Calculated for C₁₆H₂₃BrN₂O₃ (371.282): C, 51.76; H, 6.24; N, 7.55. Found: C, 51.83; H, 6.21; N, 7.52.

### Preparation 7

### Synthesis of (S)-3-(2-Phenoxy-pyridin-3-yloxymethyl)-piperidine-1-carboxylic acid tert-butyl ester

To a stirring suspension of potassium tert-butoxide (229 mg, 2.04 mmol) and 1,2-dimethoxyethane (DME, 5 mL) was added phenol (192 mg. 2.04 mmol) at room temperature. A slight temperature rise was observed and the suspension changed to a clear solution. A solution of (S)-3-(2-bromo-pyridin-3-yloxymethyl)-piperidine-1-carboxylic acid tert-butyl ester (631 mg, 1.70 mmol, Preparation 6) in 4 mL of 1,2-dimethoxyethane was then added to the reaction mixture. A catalytic amount of copper (I) trifluoromethylsulfonate (approximately 20 mg) was added to the mix and the vial was capped and heated to 100° C. for 16 hours. The mixture was rotary evaporated to remove most of the 1,2-dimethoxyethane and resuspended in water (10 mL) and diethyl ether (10 mL). This biphasic mixture was filtered through a pad of diatomaceous earth. The layers were separated and the aqueous layer was extracted with diethyl ether (2 times 50 mL). The combined organic layers were washed with 2 N NaOH (2 times 50 mL) and brine (50 mL). The organic extract was dried (Na₂SO₄), filtered, rotary evaporated to give a residue, which was chromatographed (MPLC, silica gel, 3% EtOAc in CH₂Cl₂) to give 475 mg (73%) of (S)-3-(2-phenoxy-pyridin-3-yloxymethyl)-piperidine-1-carboxylic acid tert-butyl ester as a yellow oil. MS (APCl+) m/z 385.2 [M+1, 100%], 329.2 [M−55, 56%] and 285.1 [M−99, 97%].

### Preparation 8

### Synthesis of (S)-3-(2-benzyloxy-pyridin-3-yloxymethyl)-piperidine-1-carboxylic acid, tart-butyl ester

To a stirring suspension at 0° C., under N₂ of sodium hydride (0.29 g, 7.25 mmol, 60% dispersion in mineral oil) in 5 mL of DME was added dropwise benzyl alcohol (0.75 mL, 7.25 mmol). The ice-bath was removed and the sample was stirred for 1 hour. A solution of (S)-3-(2-bromo-pyridin-3-yloxymethyl)-piperidine-1-carboxylic acid tert-butyl ester (1.80 g, 4.85 mmol, Preparation 6) in 5 mL of DME was added followed by a catalytic amount (about 50 mg) of copper(I) trifluoromethanesulfonate benzene or toluene complex (2:1). The sample was heated at 100° C. for 24 hours, cooled to room temperature, then partitioned between ethyl acetate and saturated KH₂PO₄ solution (about 50 mL of each). The organic extract was washed with brine solution, dried (MgSO₄), filtered, rotary evaporated to a residue, which was chromatographed (MPLC, silica gel, 20% EtOAc in hexanes) to give 1.30 g (68%) of (S)-3-(2-benzyloxy-pyridin-3-yloxymethyl)-piperidine-1-carboxylic acid, tert-butyl ester as a light yellow oil. MS (APCl+) m/z 399.2 [M+1, 12%], 343.2 [M−55, 3%] and 299.2 [M−99, 100%].

### Preparation 9

### Synthesis of (S)-3-(2-iodo-6-methyl-pyridin-3-yloxymethyl)-piperidine-1-carboxylic acid tert-butyl ester

To a stirring mixture at room temperature of (S)-3-hydroxymethyl-piperidine-1-carboxylic acid, tert-butyl ester (3.25 g, 15.1 mmol), 6-iodo-2-picolin-5-ol (4.00 g, 17.0 mmol) and triphenylphosphine (4.75 g, 18.1 mmol) in 15 mL of 1,2-dimethoxyethane was added dropwise diisopropyl azodicarboxylate (3.57 mL, 18.1 mmol). Addition was exothermic, after which all solid was in solution. The solution was heated at 40° C. under N₂ for 24 hours, rotary evaporated to remove most of the 1,2-dimethoxyethane, then suspended into 200 mL of diethyl ether. The solid that formed was removed by filtration. The filtrate was rotary evaporated and the resulting residue was chromatographed (MPLC, silica gel, 4% EtOAc in CH₂Cl₂) to give 6.36 g (97%) of (S)-3-(2-iodo-6-methyl-pyridin-3-yloxymethyl)-piperidine-1-carboxylic acid tert-butyl ester as a yellow solid. MS (APCl+) m/z 433.0 [M+1, 2%], 377.0 [M−55, 100%] and 333.0 [M−99, 23%].

### Preparation 10

### Synthesis of (S)-3-(6-methyl-2-phenoxy-pyridin-3-yloxymethyl)-piperidine-1-carboxylic acid tert-butyl ester

To a stirring suspension of (S)-3-(2-iodo-6-methyl-pyridin-3-yloxymethyl)-piperidine-1-carboxylic acid tert-butyl ester (714 mg, 1.65 mmol, Preparation 9), phenol (192 mg. 2.04 mmol) in 1,2-dimethoxyethane (5 mL) was added potassium tert-butoxide (229 mg, 2.04 mmol) at room temperature. A slight temperature rise was observed. A catalytic amount of copper (I) trifluoromethylsulfonate benzene complex (about 20 mg) was added to the mixture and the vial was capped and heated to 100° C. for 16 hours. The mixture was rotary evaporated to remove most of the 1,2-dimethoxyethane and resuspended into water (10 mL) and diethyl ether (10 mL). This biphasic mixture was filtered through a pad of diatomaceous earth. The layers were separated and the aqueous layer was extracted with diethyl ether (2 times 50 mL). The combined organic layers were washed with 2N NaOH (2 times 50 mL) and brine (25 mL). The organic extract was dried (Na₂SO₄), filtered, rotary evaporated to a residue, which was chromatographed (MPLC, silica gel, 20% EtOAc in hexanes) to give 564 mg (86%) of (S)-3-(6-methyl-2-phenoxy-pyridin-3-yloxymethyl)-piperidine-1-carboxylic acid tert-butyl ester as a yellow oil. MS (APCl+) m/z 399.2 [M+1, 79%], 343.2 [M−55, 15%] and 299.1 [M−99, 100%].

### Preparation 11

### Synthesis of (S)-3-formyl-piperidine-1-carboxylic acid tert-butyl ester

According to the procedure by Finney and More (Org. Lett., 2002; 4:3001), to a vigorously stirring solution of (S)-3-hydroxymethyl-piperidine-1-carboxylic acid, tert-butyl ester (5.00 g, 23.2 mmol) and ethyl acetate (160 mL) was added o-iodoxybenzoic acid (IBX, 19.5 g, 69.7 mmol). The reaction mixture was heated and refluxed for 3 hours, then allowed to cool to room temperature. The white solid was removed by filtration and the filtrate was rotary evaporated to give 4.95 g (100%) of (S)-3-formyl-piperidine-1-carboxylic acid tert-butyl ester as a colorless liquid. This was immediately carried on to the next step.

### Preparation 12

### Synthesis of (S)-3-[(S)-1-hydroxy-propyl]-piperidine-1-carboxylic acid tert-butyl ester

According to the published procedure by Knochel et al. (Tetrahedron, 1998, 54, 6385), to a stirring solution of (1R)-trans-N,N′-1,2-cyclohexanediylbis(1,1,1-trifluoromethanesulfonamide) (702 mg, 1.86 mmol) and dry diethyl ether (30 mL) under N₂ was added titanium (IV) isopropoxide (8.16 mL, 27.9 mmol) via syringe. The reaction mixture was cooled to −15° C. with an ice/NaCl bath. To the cold mixture was added diethyl zinc (5.71 mL, 55.7 mmol) and a bright yellow solution was formed which stirred for 45 minutes. (S)-3-Formyl-piperidine-1-carboxylic acid tert-butyl ester (4.95 g, 23.2 mmol, Preparation 11) was dissolved in 20 mL of dry diethyl ether and added to the reaction mixture (via cannula) dropwise over 5 minutes. The reaction was then placed in a minus 20° C. freezer for 16 hours, then diluted with diethyl ether (50 mL) and quenched carefully with sat. NH₄Cl solution. Then 1N HCl (100 mL) was added to dissolve the solids and then the mixture was extracted with diethyl ether (3 times 50 mL). The combined organics were washed with 2N NaOH (100 mL) and brine (100 mL), dried (Na₂SO₄), filtered and rotary evaporated. The residue was chromatographed (MPLC, silica gel, 10% EtOAc in CH₂Cl₂) to give 4.34 g (79%) of (S)-3-[(S)-1-hydroxy-propyl]-piperidine-1-carboxylic acid tert-butyl ester as a colorless oil. MS (APCl+) m/z 244.1 [M+1, 10%], 188.1 [M−55, 100%] and 144.0 [M−99, 38%].

### Preparation 13

### Synthesis of (S)-3-[(R)-1-benzoyloxy-propyl]-piperidine-1-carboxylic acid tert-butyl ester

To a solution at 0° C. of (S)-3-[(S)-1-hydroxy-propyl]-piperidine-1-carboxylic acid tert-butyl ester (3.55 g, 14.6 mmol, Preparation 12), triphenylphosphine (15.0 g, 58.0 mmol), benzoic acid (7.1 g, 58 mmol), diisopropylethylamine (10.2 mL, 58.4 mmol) and 1,2-dimethoxyethane (100 mL) was added diisopropyl azodicarboxylate (11.5 ml, 58.4 mmol) dropwise via syringe. The reaction was heated at 45° C. for 16 hours, rotary evaporated to about ½ volume, and diluted with 120 mL of a hexanes:diethyl ether mixture (5:1). The precipitate that formed was removed by filtration and the filtrate was diluted with 300 mL of diethyl ether. This was washed with 1N HCl (200 mL), water (100 mL), sat NaHCO₃ (100 mL) and brine (100 mL) solutions, dried (Na₂SO₄), filtered, rotary evaporated to a residue, which was chromatographed (MPLC, silica gel, 10% EtOAc in hexanes) to give 3.35 g (66%) of (S)-3-[(R)-1-benzoyloxy-propyl]-piperidine-1-carboxylic acid tert-butyl ester as a slightly yellow oil. This oil was taken up in a minimal amount of pentane and allowed to crystallize at −20° C. to give 2.83 g of colorless needles. MS (APCl+) m/z 348.2 [M+1, 5%], 292.2 [M−55, 51%] and 248.2 [M−99, 100%].

### Preparation 14

### Synthesis of (S)-3-[(R)-1-hydroxy-propyl]-piperidine-1-carboxylic acid tert-butyl ester

To a stirring solution of sodium hydroxide (1.30 g, 32.6 mmol) and methanol (165 mL) was added (S)-3-[(R)-1-benzoyloxy-propyl]-piperidine-1-carboxylic acid tert-butyl ester (2.83 g, 8.15, Preparation 13). The mixture was heated to reflux for 1 hour, cooled to room temperature, rotary evaporated and diluted with water (100 mL). The aqueous mixture was extracted with ethyl ether (2 times 100 mL), washed with saturated NaHCO₃ (100 mL) solution, dried (Na₂SO₄), filtered and rotary evaporated to give 1.98 g (100%) of (S)-3-[(R)-1-hydroxy-propyl]-piperidine-1-carboxylic acid tert-butyl ester as a colorless oil. MS (APCl+) m/z 244.1 [M+1, 15%], 188.1 [M−55, 100%] and 144.0 [M−99, 23%].

### Preparation 15

### Synthesis of (S,S)-3-[1-(2-bromo-pyridin-3-yloxy)-propyl]-piperidine-1-carboxylic acid tert-butyl ester

Following a process analogous to Preparation 6, (S)-3-[(R)-1-hydroxy-propyl]-piperidine-1-carboxylic acid tert-butyl ester was converted to 2.50 g (77%) of (S,S)-3-[1-(2-bromo-pyridin-3-yloxy)-propyl)-piperidine-1-carboxylic acid tert-butyl ester as a yellow oil. MS (APCl+) m/z 401.0 [M+1, 96%], 344.9 [M−55, 100%] and 299.0 [M−99, 70%] (all exist as doublets from the presence of the bromo functionality).

### Preparation 16

### Synthesis of (S,S)-3-[1-(2-phenoxy-pyridin-3-yloxy)-propyl]-piperidine-1-carboxylic acid tert-butyl ester

This compound was synthesized using a process analogous to Preparation 7 to give 486 mg (82%) of (S,S)-3-[1-(2-phenoxy-pyridin-3-yloxy)-propyl]-piperidine-1-carboxylic acid tert-butyl ester as a yellow oil. MS (APCl+) m/z 413.2 [M+1, 78%], 357.1 [M−55, 47%] and 313.2 [M−99, 100%].

### Preparation 17

### Synthesis of (S)-3-(methoxy-methyl-carbamoyl)-piperidine-1-carboxylic acid, tert-butyl ester

To a stirring solution at −78° C. under N₂ of piperidine-1,3-dicarboxylic acid 1-tert-butyl ester (N-BOC-(S)-nipecotic acid, 15.0 g, 65.6 mmol) and 1-methylpiperidine (9.6 mL, 79.0 mmol) in 500 mL of CH₂Cl₂ was added rapidly (via syringe) ethyl chloroformate (6.9 mL, 72.2 mmol). The mixture (solid had formed) was stirred for 15 minutes, then solid N,O-dimethylhydroxylamine hydrochloride (7.0 g, 71.8 mmol) followed by another portion of 1-methylpiperidine (9.6 mL, 79.0 mmol) were added. The sample was allowed to slowly warm to room temperature (˜4 hours), rotary evaporated, then partitioned between EtOAc and saturated NaHCO₃ solution. The organic extract was washed with sat. KH₂PO₄ and brine solutions, dried (MgSO₄), filtered and rotary evaporated to give 18.2 g (>100%) of (S)-3-(methoxy-methyl-carbamoyl)-piperidine-1-carboxylic acid, fed-butyl ester as colorless oil. MS (APCl+) m/z 173.1 [M−99, 100%]. This material was used without further purification in Preparation 18.

### Preparation 18

### Synthesis of (S)-3-benzoyl-piperidine-1-carboxylic acid tert-butyl ester

A solution of 3M phenyl magnesium bromide in diethyl ether solution (30.6 mL, 91.8 mmol) was added dropwise to a stirring solution at 0° C. under N₂ of (S)-3-(methoxy-methyl-carbamoyl)-piperidine-1-carboxylic acid, tert-butyl ester (18.2 g, entire sample from Preparation 17 assume 65.5 mmol) in 300 mL of THF. The solution was stirred for 1 hour, then quenched by dropwise addition of 250 mL of saturated KH₂PO₄ solution. The reaction was allowed to warm to room temperature (˜4 hours), rotary evaporated to remove most of the THF, then extracted with ethyl acetate. The organic extract was washed with brine solution, dried (MgSO₄), filtered, rotary evaporated, and chromatographed (MPLC, silica gel, 20% EtOAc in hexanes) to give 15.3 g (81%) of (S)-3-benzoyl-piperidine-1-carboxylic acid tert-butyl ester as a light yellow oily-solid. A portion was crystallized from hexanes to give a white solid, mp 75-79° C. Elemental Analysis: Calculated for C₁₇H₂₃NO₃ (289.378): C, 70.56; H, 8.01; N, 4.84. Found: C, 70.48; H, 8.08; N, 4.78.

### Preparation 19

### Synthesis of (S)-3-[(R,S)-hydroxy-phenyl-methyl]-piperidine-1-carboxylic acid tert-butyl ester

A solution of (S)-3-benzoyl-piperidine-1-carboxylic acid tert-butyl ester (8.0 g, 27.6 mmol, Preparation 18) in 100 mL of MeOH was added dropwise to a stirring suspension at 0° C. under N₂ of sodium borohydride (5.2 g, 137.7 mmol) in 100 mL of methanol containing 10 mL of 1N NaOH solution. The sample was allowed to slowly warm to room temperature overnight, rotary evaporated to remove most of the MeOH, then partitioned between ethyl acetate and 10% aqueous NH₄OH solution. The organic extract was washed with saturated KH₂PO₄ and brine solutions, dried (MgSO₄), filtered, rotary evaporated, and chromatographed (MPLC, silica gel, 20% EtOAc in hexanes) to give 8.2 g (>100%) of a mixture of diastereomers (about 1:1 by ¹H-NMR) of (S)-3-[(R,S)-hydroxy-phenyl-methyl]-piperidine-1-carboxylic acid tert-butyl ester as a colorless oil. MS (APCl+) m/z 192.1 [M−99, 100%]. This sample contains solvent and was used without further purification in Preparation 20.

### Preparation 20

### Synthesis of 3-[(2-bromo-pyridin-3-yloxy)-phenyl-methyl]-(S)-piperidine-1-carboxylic acid tert-butyl ester, stereoisomer A and stereoisomer B

To a stirring mixture at room temperature under N₂ of (S)-3-[(R,S)-hydroxy-phenyl-methyl]-piperidine-1-carboxylic acid tert-butyl ester (8.2 g, 28.1 mmol, Preparation 19), 2-bromo-3-pyridinol (6.1 g, 35.1 mmol) and triphenylphosphine (9.2 g, 35.1 mmol) in 50 mL of DME was added dropwise diisopropyl azodicarboxylate (6.9 mL, 35.0 mmol). The reaction was stirred at room temperature for 24 hours, rotary evaporated, and redissolved into diethyl ether. The solution was washed with 1N NaOH (2×), saturated KH₂PO₄ and brine solutions, dried (MgSO₄), filtered and rotary evaporated to a dark yellow oil. The sample was first chromatographed (MPLC, silica gel, 10% EtOAc in CH₂Cl₂) to remove the Mitsunobu reaction by-products then chromatographed again (MPLC (2×), silica gel, 20% EtOAc in hexanes) to obtain individual diastereomers of 3-[(2-bromo-pyridin-3-yloxy)-phenyl-methyl]-(S)-piperidine-1-carboxylic acid tert-butyl ester.

Stereoisomer A: 3.7 g (29%) as a light yellow foamy solid. R_{f}=0.26 (silica gel, 20% EtOAc in hexanes). MS (APCl+) m/z 347/349 [M−99, 93/100%].

Stereoisomer B: 2.9 g (23%) as a light yellow foamy hygroscopic solid. R_{f}=0.22 (silica gel, 20% EtOAc in hexanes). MS (APCl+) m/z 347/349 [M−99, 93/100%].

### Preparation 21

### Synthesis of 3-{[2-(4-fluoro-phenoxy)-pyridin-3-yloxy]-phenyl-methyl}-(S)-piperidine-1-carboxylic acid tert-butyl ester, stereoisomer A

To a room temperature solution of 4-fluorophenol (0.47 g, 4.2 mmol) in 5 mL of DME in a vial was added potassium tert-butoxide (0.47 g, 4.2 mmol). The sample was stirred for 30 minutes, then a solution of 3-[(2-bromo-pyridin-3-yloxy)-phenyl-methyl]-(S)-piperidine-1-carboxylic acid tert-butyl ester, stereoisomer A (1.25 g, 2.8 mmol, Preparation 20) in 5 mL of DME followed by a catalytic amount (about 50 mg) of copper (I) trifluoromethanesulfonate benzene complex (2 to 1) were added. The sample vial was sealed and heated at 100° C. (via a block heater) for 24 hours, then room temperature. The sample was partitioned between ethyl acetate and 1N NaOH solution. The organic extract was washed with another portion of 1N NaOH, saturated KH₂PO₄ and brine solutions, dried (MgSO₄), filtered and rotary evaporated. Chromatography (MPLC, silica gel, 20% EtOAc in hexanes) gave 1.13 g (84%) of 3-{[2-(4-fluoro-phenoxy)-pyridin-3-yloxy]-phenyl-methyl}-(S)-piperidine-1-carboxylic acid tert-butyl ester, stereoisomer A as a foamy white solid. MS (APCl+) m/z 379.1 [M−99, 100%].

### Preparation 22

### Synthesis of (S)-3-methanesulfonylmethyl-piperidine-1-carboxylic acid tert-butyl ester

(S)-3-Hydroxymethyl-piperidine-1-carboxylic acid tert-butyl ester (10.2 g, 0.047 mol) was dissolved in 500 mL dichloromethane, and the solution stirred under N₂ in an ice bath. Triethylamine (6.2 g, 0.061 mol) and methanesulfonyl chloride (6.5 g, 0.057 mol) were added sequentially. After about 0.5 hour, the ice bath was removed. After about 3 hours total reaction time, the reaction mixture was washed with aqueous acid, aqueous base, and brine, then filtered through sodium sulfate and rotary evaporated in vacuo to an oil, which solidified on standing, to give 14 g of the title compound.

### Preparation 23

### Synthesis of (S)-3-[2-(4-chloro-2-fluoro-phenoxy)-6-methyl-pyridin-3-yloxymethyl]-piperidine-1-carboxylic acid tert-butyl ester

(S)-3-(2-Iodo-6-methyl-pyridin-3-yloxymethyl)-piperidine-1-carboxylic acid tert-butyl ester (0.128 g, 0.296 mmol, Preparation 1) and 4-chloro-2-fluorophenol (0.087 g, 0.59 mmol) were charged to an 8 mL septum-capped vial with stir bar and purged with nitrogen. 1,2-dimethoxyethane (0.6 mL) and potassium tert-butoxide/tetrahydrofuran solution (1 M, 0.59 mL) were added via syringe, followed by about 10 mg of copper (I) triflate-toluene complex. The vial was placed in a dry block heated at 100° C. on a stirrer/hot plate for 18-24 hours. The reaction mixture was chromatographed on silica gel, eluting with a linear gradient 0-40% ethyl acetate and 100-60% hexanes to yield (S)-3-[2-(4-chloro-2-fluoro-phenoxy)-6-methyl-pyridin-3-yloxymethyl]-piperidine-1-carboxylic acid tert-butyl ester as an oil (107 mg).

### Preparation 24

### Synthesis of (S)-3-[2-(4-chloro-2,6-difluoro-phenoxy)-pyridin-3-yloxymethyl]-6-methyl piperidine-1-carboxylic acid tert-butyl ester

(S)-3-(2-Iodo-6-methyl-pyridin-3-yloxymethyl)-piperidine-1-carboxylic acid tert-butyl ester (0.60 g, 1.4 mmol, Preparation 1), 4 chloro-2,6-difluoro-phenol (0.32 g, 19 mmol), and pyridine (oxygen free) (12 mL) were charged to an 35 ml thick walled pressure tube equipped with a stir bar. The mixture was stirred, and cesium carbonate (0.87 g, 2.57 mmol) was added, followed by copper (I) triflate benzene complex (0.07 g, 0.12 mmol). The sealed reaction vessel was heated to 120 C on an oil bath. The reaction mixture was chromatographed on silica gel, using hexane/ethyl acetate as a mobile phase. The correct fractions where combined and the solvent removed under reduce pressure to afford the title compound as an oil (0.185 g, 28%).

### Preparation 25

### Synthesis of (S)-3-(2-benzyloxy-phenoxymethyl)-piperidine-1-carboxylic acid tert-butyl ester

To a stirring mixture at room temperature of (S)-3-hydroxymethyl-piperidine-1-carboxylic acid, tert-butyl ester (2.65 g, 12.31 mmol), 2-(benzyloxy)-phenol (2.4 mL, 13.70 mmol), and triphenylphosphine (4.04 g, 15.40 mmol) in 20 mL of 1,2-dimethoxyethane was added drop wise diisopropyl azodicarboxylate (3.1 mL, 15.74 mmol). Addition was exothermic, after which all solid was in solution. The solution was heated at 50° C. under N₂ for 24 hours, rotary evaporated (to remove most of the 1,2-dimethoxyethane), then suspended into 75 mL of hexanes. The solid that formed was removed by filtration. The filtrate was rotary evaporated and chromatographed (MPLC, silica gel, 100% CH₂Cl_{2 [}2L] then 20% EtOAc in hexanes [2L]) to give 3.96 g (81%) of (S)-3-(2-benzyloxy-phenoxymethyl)-piperidine-1-carboxylic acid tert-butyl ester as light yellow oil. MS (APCl⁺) m/z 298.2 [M−99, 100%].

### Preparation 26

### Synthesis of (S)-3-(4-fluoro-2-phenoxy-phenoxymethyl)-piperidine-1-carboxylic acid tert-butyl ester

A mixture of (S)-3-(4-fluoro-2-hydroxy-phenoxymethyl)-piperidine-1-carboxylic acid tert-butyl ester (0.91 g, 2.79 mmol, prepared in a manner analogous to the method of Preparation 27), phenylboronic acid (0.68 g, 5.58 mmol), copper (II) acetate (0.51 g, 2.81 mmol), pyridine (1.1 mL, 13.97 mmol) and powdered 4 Å activated molecular sieves (˜5 g) in 27 mL of CH₂Cl₂ was stirred at room temperature under ambient atmosphere for 24 hours. The sample was filtered through a pad of diatomaceous earth, rotary evaporated then partitioned between EtOAc and 1 N NaOH solution. The organic extract was washed with sat. KH₂PO₄ and brine solutions, dried (MgSO₄), filtered, rotary evaporated and chromatographed (MPLC, silica gel, 20% EtOAc in hexanes) to give 0.74 g (66%) of (S)-3-(4-fluoro-2-phenoxy-phenoxymethyl)-piperidine-1-carboxylic acid tert-butyl ester as light yellow oil. MS (APCl⁺) m/z 402.1 [M+1, 17.4%] and 302.5 [M−99, 100%].

### Preparation 27

### Synthesis of (S)-3-(2-hydroxy-phenoxymethyl)-piperidine-1-carboxylic acid tert-butyl ester

A solution of (S)-3-(2-benzyloxy-phenoxymethyl)-piperidine-1-carboxylic acid tert-butyl ester (Preparation 25, 3.24 g, 8.17 mmol) in 100 mL of ethanol was treated with 0.60 g of 20% Pd/C. The sample was hydrogenated at room temperature under balloon pressure for 1 hour, filtered and rotary evaporated to give 2.44 g (97%) of (S)-3-(2-hydroxy-phenoxymethyl)-piperidine-1-carboxylic acid tert-butyl ester as off-white solid, mp 98-101° C. Elemental Analysis: Calculated for C₁₇H₂₅NO₄ (307.393): C, 66.43; H, 8.20; N, 4.56. Found: C, 66.27; H, 8.60; N, 4.50. MS (APCl+) m/z 208.1 [M−99, 100%].

### Preparation 28

### Synthesis of (S)-3-(2-cyclohexyloxy-phenoxymethyl)-piperidine-1-carboxylic acid tert-butyl ester

To a stirring mixture at room temperature of (S)-3-(2-hydroxy-phenoxymethyl)-piperidine-1-carboxylic acid tert-butyl ester (Preparation 27, 1.00 g, 3.25 mmol), cyclohexanol (0.51 mL, 4.83 mmol) and triphenylphosphine (1.28 g, 4.88 mmol) in 10 mL of THF was added drop wise diisopropyl azodicarboxylate (0.96 mL, 4.88 mmol). Addition was exothermic, after which all solid was in solution. The sample was sealed and heated at 50° C. (via a block heater). TLC after 72 hours still showed starting material present. Another portion of cyclohexanol (0.51 mL), triphenylphosphine (1.28 g) and DIAD (0.96 mL) were added and heated at 50° C. for 24 hours. The sample was cooled, rotary evaporated then suspended into 75 mL of hexanes. The solid that formed was removed by filtration. The filtrate was rotary evaporated and chromatographed (MPLC, silica gel, 20% EtOAc in hexanes) to give 0.75 g (59%) of (S)-3-(2-cyclohexyloxy-phenoxymethyl)-piperidine-1-carboxylic acid tert-butyl ester as colorless oil. MS (APCl⁺) m/z 290.2 [M−99, 100%].

### Preparation 29

### Synthesis of (S)-3-[(2-ethoxy-phenoxy)-(R,S)-phenyl-methyl]-piperidine-1-carboxylic acid tert-butyl ester

A mixture of (S)-3-[(R,S)-hydroxy-phenyl-methyl]-piperidine-1-carboxylic acid tert-butyl ester (1.53 g, 5.24 mmol, Preparation 19), 2-ethoxyphenol (0.83 mL, 6.55 mmol), triphenylphosphine (1.72 g, 6.56 mmol) and diisopropyl azodicarboxylate (1.3 mL, 6.60 mmol) in 10 mL of THF was heated at 60° C. for 24 hours. The sample was cooled, rotary evaporated then suspended into 75 mL of hexanes. The solid that formed was removed by filtration. The filtrate was rotary evaporated and chromatographed (MPLC, silica gel, 100% CH₂Cl₂ [2L] then 20% EtOAc in hexanes [2L]) to give 0.99 g (46%) of (S)-3-[(2-Ethoxy-phenoxy)-(R,S)-phenyl-methyl]-piperidine-1-carboxylic acid tert-butyl ester as colorless oil. MS (APCl⁺) m/z 312.2 [M−99, 100%].

### Preparation 30

### Synthesis of (S)-3-acetyl-piperidine-1-carboxylic acid tert-butyl ester

To a solution stirring solution at 0° C. of piperidine-1,3-carboxylic acid, 1-tert-butyl ester (N-Boc-(S)-nipecotic acid, 7.20 g, 26.4 mmol, Preparation 17) in tetrahydrofuran (20 mL) was added 3.0 M methylmagnesium bromide in diethyl ether (12.5 mL, 37.5 mmol, 1.4 eq.). After stirring at 0° C. for 30 minutes, a saturated solution of ammonium chloride (10 mL) was added and the mixture was extracted with ethyl acetate (2 times 25 mL). The combined organics were dried (Na₂SO₄), filtered and rotary evaporated. The resulting oil was chromatographed (MPLC, silica gel, 8% EtOAc in hexanes) to give 4.86 g (81%) of (S)-3-acetyl-piperidine-1-carboxylic acid tert-butyl ester as a yellow oil with an enantiomeric excess of 94% (HPLC, CHIRALPAK® AD-H (Chiral Technologies, Inc., Exton, Pa.), 20% ethanol in hexanes with 0.1% TFA).

### Preparation 31

### Synthesis of 3-(1-hydroxy-ethyl)-piperidine-1-carboxylic acid tert-butyl ester

A stirring solution of 1 M (S)-2-methyl-CBS-oxazoborolidine (Chemical Abstracts No. 112022-81-8, 262 L, 0.262 mmol, 0.11 eq.) in toluene (5 mL) was placed in a room temperature water bath to control the internal temperature. N,N-diethylaniline borane (472 L, 2.65 mmol, 1.1 eq.) was added drop wise via syringe to the stirring solution. (S)-3-Acetyl-piperidine-1-carboxylic acid tert-butyl ester (Preparation 30) was dissolved in toluene (2 mL) and added dropwise via cannula to the reaction mixture over 30 minutes. The reaction was allowed to stir for 1 hour before an aliquot was quenched and checked by TLC. The completed reaction was quenched with methanol (5 mL—CAUTION—gas evolution), diluted with 1 N HCl (10 mL) and allowed to stir for 5 minutes then extracted with diethyl ether (3 times 20 mL). The combined organic layers were washed with 0.5 N HCl (2 times 10 mL), water (10 mL) and brine solution (20 mL). The organic layer was dried (Na₂SO₄), filtered and rotary evaporated. 531 mg (94%) of 3-(1-hydroxy-ethyl)-piperidine-1-carboxylic acid tert-butyl ester was isolated as a yellow oil with an enantiomeric excess of 88% (HPLC, CHIRALPAK® AD-H, 20% ethanol in hexanes with 0.1% TFA).

### Preparation 32

### Synthesis of (S)-3-(1-Hydroxy-1-methyl-ethyl)-piperidine-1-carboxylic acid tert-butyl ester

To a stirring solution of (S)-piperidine-1,3-dicarboxylic acid 1-tert-butyl ester 3-ethyl ester (ethyl (S)-1-Boc-nipecotate, 3.00 g, 11.7 mmol) in tetrahydrofuran (30 mL) at 0° C., 3 M methylmagnesium bromide in diethyl ether (9.0 mL, 27 mmol, 2.3 eq.) was added via syringe. The reaction was stirred and allowed to warm to room temperature overnight. The reaction was quenched with a saturated solution of ammonium chloride (100 mL) and extracted with dichloromethane (2 times 100 mL). The organic layer was dried (Na₂SO₄), filtered and rotary evaporated to give 2.74 g (96%) of (S)-3-(1-hydroxy-1-methyl-ethyl)-piperidine-1-carboxylic acid tert-butyl ester as a yellow oil. MS (APCl+) m/z 170.0 [M−73, 100%], 144.0 [M−99, 10%].

### Preparation 33

### Synthesis of tert-Butyl-(2-fluoro-benzylidene)-amine

To a stirring solution of 2-fluorobenzaldehyde (8.5 mL, 80 mmol) in benzene (50 mL) was added tert-butylamine (15 mL, 120 mmol, 1.5 eq.). The reaction flask was equipped with a Dean-Stark trap and heated to reflux overnight. The reaction was allowed to cool to room temperature and then was rotary evaporated to give 12.24 g (85%) of tert-butyl-(2-fluoro-benzylidene)-amine as a pale orange oil that was sufficiently pure enough to carry on. MS (APCl+) m/z 180.1 [M+1, 3%], 123.9 [M−55, 100%].

### Preparation 34

### Synthesis of (S)-3-[1-(2-Formyl-phenoxy)-1-methyl-ethyl]-piperidine-1-carboxylic acid tert-butyl ester

To a stirring solution of (S)-3-(1-hydroxy-1-methyl-ethyl)-piperidine-1-carboxylic acid tert-butyl ester (Preparation 32, 6.56 g, 27.0 mmol) in dioxane (18 mL) at 0° C. was added sodium hydride (1.19 g, 29.7 mmol, 1.1 eq.) in four portions. The reaction was allowed to stir for 15 minutes then warmed to room temperature and stirred an additional hour. tert-Butyl-(2-fluoro-benzylidene)-amine (Preparation 33, 7.26 g, 40.5 mmol, 1.5 eq.) was then added and the reaction mixture was equipped with a condenser and heated to reflux temperature overnight. The reaction was allowed to cool to room temperature and was quenched with a saturated solution of monobasic potassium phosphate (50 mL). The mixture was extracted with ethyl acetate (2 times 100 mL) and the organic layer was dried (Na₂SO₄), filtered and rotary evaporated to yield a brown gum. The product was dissolved in acetic acid (35 mL), water (100 mL) and tetrahydrofuran (50 mL) and allowed to stir overnight. The mixture was extracted with ethyl acetate (2 times 200 mL) and the combined organic layers were washed with water (2 times 100 mL) and brine solution (100 mL). The organic layer was dried (Na₂SO₄), filtered and rotary evaporated. The crude product was purified by chromatography (MPLC, silica gel, 2.5% EtOAc in dichloromethane) to give 2.48 g (26%) of (S)-3-[1-(2-formyl-phenoxy)-1-methyl-ethyl]-piperidine-1-carboxylic acid tert-butyl ester as a white solid. MS (APCl+) m/z 248.0 [M−99, 8%], 170.0 [M−177, 100%].

### Preparation 35

### Synthesis of (S)-3-(2-fluoro-6-hydroxy-phenoxymethyl)-piperidine-1-carboxylic acid tert-butyl ester

To a stirring solution of (S)-3-(2-fluoro-6-methoxy-phenoxymethyl)-piperidine-1-carboxylic acid tert-butyl ester (Example 103, 2.70 g, 7.96 mmol) in 1-methyl-2-pyrrolidone (25 mL) was added sodium thioethoxide (1.49 g, 15.9 mmol, 2.0 eq.). The reaction was equipped with a reflux condenser and heated to 100° C. for 8 hours and then allowed to cool to room temperature. The mixture was extracted with diethyl ether (2 times 100 mL) and the combined organic layers were washed with water (2 times 100 mL) and brine solution (100 mL). The organic layer was dried (Na₂SO₄), filtered and rotary evaporated to give 2.49 g (96%) of (S)-3-(2-fluoro-6-hydroxy-phenoxymethyl)-piperidine-1-carboxylic acid tert-butyl ester as a pale yellow oil.

### Preparation 36

### Synthesis of (S)-3-(pyridine-2-carbonyl)-piperidine-1-carboxylic acid tert-butyl ester

To a stirring solution of 2-bromopyridine (5.06 g, 32.0 mmol, 1.3 eq.) in tetrahydrofuran (45 mL) at −78° C. was added 2.49 M n-butyl lithium in hexanes (13.4 mL, 33.3 mmol, 1.33 eq.) dropwise via syringe. The solution turned to a deep red color and was allowed to stir for 15 minutes. In a separate flask, (S)-3-(2-methoxy-propionyl)-piperidine-1-carboxylic acid tert-butyl ester (6.71 g, 24.6 mmol) was dissolved in tetrahydrofuran (30 mL). This solution was then cannulated into the reaction flask drop wise over 15 minutes and allowed to stir for 1 hour at −78° C. The reaction was quenched with a saturated solution of monobasic potassium phosphate (50 mL) and extracted with diethyl ether (2 times 100 mL). The combined organic layers were washed with brine (100 mL), dried (Na₂SO₄), filtered and rotary evaporated. The crude product was purified by chromatography (MPLC, silica gel, 20% hexanes in ethyl acetate) to give 4.76 g (67%) of (S)-3-(pyridine-2-carbonyl)-piperidine-1-carboxylic acid tert-butyl ester as a yellow oil. MS (APCl+) m/z 191.0 [M−99, 37%], 173.0 [M−117, 100%].

### Preparation 37

### Synthesis of (S)-3-((S)-hydroxy-pyridin-2-yl-methyl)-piperidine-1-carboxylic acid tert-butyl ester

(S)-3-(pyridine-2-carbonyl)-piperidine-1-carboxylic acid tert-butyl ester (Preparation 36, 4.75 g, 16.4 mmol), potassium carbonate (0.564 g, 4.1 mmol, 0.25 eq.), dichloro[(S)-(−)-2,2′-bis(diphenylphosphino)-1,1′-binaphthyl][(2S)-(+)-1,1-bis(4-methoxyphenyl)-3-methyl-1,2-butanediamine]ruthenium (II) (0.036 g, 0.033 mmol, 0.002 eq. Strem Chemical Co.), isopropanol (80 mL) and tetrahydrofuran (20 mL) were sealed in a pressure reactor inside a glove box. The reactor was pressurized with 50 psi of H₂ and stirred at room temperature for 16 hours. The reaction was rotary evaporated, taken up in ethyl acetate, and filtered through a pad of diatomaceous earth. The solvent was removed by rotary evaporation to give 4.55 g (95%) of (S)-3-((S)-hydroxy-pyridin-2-yl-methyl)-piperidine-1-carboxylic acid tert-butyl ester as a yellow oil with a diastereomer ratio of 16:1. The crude product was recrystallized from hexanes:diethyl ether (10:1, 11 mL) to give 3.02 g of a crystalline solid with a ratio of diastereomers of 25:1. MS (APCl+) m/z 193.0 [M−99, 100%].

### Example 1

### Synthesis of (S)-3-[2-(4-fluoro-2-methyl-phenoxy)-6-methyl-pyridin-3-yloxymethyl]-piperidine fumaric acid

(S)-3-[2-(4-Fluoro-2-methyl-phenoxy)-6-methyl-pyridin-3-yloxymethyl]-piperidine-1-carboxylic acid tert-butyl ester (270 mg, 0.63 mmol) from Preparation 2 was dissolved in 3.6 mL dichloromethane and cooled in an ice bath. Trifluoroacetic acid (2.4 mL) was added, and, after 45 minutes, the ice bath was removed. After 3 hours, volatiles were removed in vacuo, and the residue was partitioned between dichloromethane (15 mL) and 15% aqueous sodium hydroxide (1 mL). The organic layer was filtered through sodium sulfate and rotary evaporated in vacuo. The residue (200 mg, 0.605 mmol) was dissolved in high pressure liquid chromatography (HPLC) grade acetone (4 mL) and a solution of fumaric acid (70 mg, 0.61 mmol) in acetone (12 mL) was added in one portion. The mixture was stirred overnight and filtered. The solid was washed freely with acetone and dried in vacuo at 35° C. to give 230 mg of (S)-3-[2-(4-fluoro-2-methyl-phenoxy)-6-methyl-pyridin-3-yloxymethyl]-piperidine fumaric acid.

### Example 23

### Synthesis of (S)-2-(4-chloro-2-fluoro-phenoxy)-6-methyl-3-(piperidin-3-ylmethoxy)-pyridine fumaric acid

(S)-3-[2-(4-Chloro-2-fluoro-phenoxy)-6-methyl-pyridin-3-yloxymethyl]-piperidine-1-carboxylic acid tert-butyl ester (85 mg, 0.189 mmol, Preparation 23) was dissolved in 3 mL dichloromethane and cooled in an ice bath. Trifluoroacetic acid (2 mL) was added, and, after about 30 minutes, the ice bath was removed. After 2 hours, volatiles were removed in vacuo, and the residue was partitioned between dichloromethane (15 mL) and 15% aqueous sodium hydroxide (1 mL). The organic layer was filtered through sodium sulfate and rotary evaporated in vacuo. The residue was dissolved in HPLC-grade acetone (3 mL) and a solution of fumaric acid (21.8 mg, 0.188 mmol) in acetone (2.7 mL) was added in portions. The mixture was stirred for four days and filtered. The solid was washed freely with acetone and dried in vacuo at 35° C. to give 70.1 mg of the title compound.

### Example 45

### Synthesis of (S)2-(4-chloro-2,6-difluoro-phenoxy)-6-methyl-3-(piperidin-3-ylmethoxy)-pyridine hydrochloride

A stirred solution of (S)3-[2-(4-chloro-2,6-difluoro-phenoxy)-prridin-3-yloxymethyl]-6-methyl piperidine-1-carboxylic acid tert-butyl ester (0.185 g, 0.38 mol, Preparation 24) in dichloromethane (1.0 mL) was treated with a solution of hydrogen chloride in ether (2M, 2.0 mL) and allowed to stir for 20 hours. The resulting solid was recovered by filtration and washed with ether and hexane to afford the title compound (0.14 g, 69%).

### Example 46

### Synthesis of (S)-3-(4-Fluoro-2-phenoxy-phenoxymethyl)-piperidine fumaric acid

A solution of (S)-3-(4-fluoro-2-phenoxy-phenoxymethyl)-piperidine-1-carboxylic acid tert-butyl ester (Preparation 26, 0.74 g, 1.85 mmol) in 50 mL of CH₂Cl₂ was treated with CF₃CO₂H (5 mL). The solution was stirred at room temperature under N₂ for 2 hours, rotary evaporated then partitioned between CHCl₃ and 10% aqueous NH₄OH solution. The organic extract was washed with brine solution, dried (MgSO₄), filtered, rotary evaporated to give the free base of the titled compound as light yellow oil. The sample was converted to the fumaric acid salt and precipitated from 2-propanol (minimum amount) and CH₃CN to give 0.47 g of (S)-3-(4-fluoro-2-phenoxy-phenoxymethyl)-piperidine fumarate as white solid.

### Example 77

### Synthesis of (S)-2-benzyloxy-3-(piperidin-3-ylmethoxy)-pyridine fumaric acid

A solution of (S)-3-(2-benzyloxy-pyridin-3-yloxymethyl)-piperidine-1-carboxylic acid, tert-butyl ester (1.30 g, 3.27 mmol, Preparation 8) in 100 mL of CH₂Cl₂ was treated with 10 mL of trifluoroacetic acid. The solution was stirred at room temperature under N₂ for 2 hours, rotary evaporated, then partitioned between CHCl₃ and 10% aqueous NH₄OH solution. The organic extract was washed with brine solution, dried (MgSO₄), filtered and rotary evaporated to give 0.74 g (55%) of the free base of the title compound as a light yellow oil. The sample was converted to the fumaric acid salt and precipitated from cold 2-propanol (minimum amount) and acetonitrile to give (S)-2-benzyloxy-3-(piperidin-3-ylmethoxy)-pyridine fumaric acid as a white solid.

### Example 84

### Synthesis of (S,S)-2-Phenoxy-3-(1-piperidin-3-yl-propoxy)-pyridine fumaric acid

This compound was synthesized using a process analogous to Example 93 to give 374 mg (74%) of (S,S)-2-phenoxy-3-(1-piperidin-3-yl-propoxy)-pyridine fumaric acid as a white solid.

### Example 88

### Synthesis of 2-(4-fluoro-phenoxy)-3-[((R or S)-phenyl)-((S)-piperidin-3-yl)-methoxy]-pyridine fumaric acid, stereoisomer A

A solution of 3-{[2-(4-fluoro-phenoxy)-pyridin-3-yloxy]-phenyl-methyl}-(S)-piperidine-1-carboxylic acid tert-butyl ester, stereoisomer A (1.13 g, 2.36 mmol, Preparation 21) in 100 mL of CH₂Cl₂ was treated with 10 mL of trifluoroacetic acid. The solution was stirred at room temperature under N₂ for 2 hours, rotary evaporated, then partitioned between CHCl₃ and aqueous NH₄OH solution. The organic extract was washed with brine solution, dried (MgSO₄), filtered and rotary evaporated. The resulting light yellow oil was converted to the fumaric acid salt and crystallized from cold 2-propanol to give 1.01 g (86%) of stereoisomer A of the title compound as a white solid.

### Example 93

### Synthesis of (S)-2-Phenoxy-3-(piperidin-3-ylmethoxy)-pyridine fumaric acid

A solution of (S)-3-(2-phenoxy-pyridin-3-yloxymethyl)-piperidine-1-carboxylic acid tert-butyl ester (475 mg, 1.24 mmol, Preparation 7) in 10 mL of CH₂Cl₂ was treated with 2 mL of trifluoroacetic acid. The solution was stirred at room temperature under N₂ for 3 hours, rotary evaporated then partitioned between CH₂Cl₂ and 10% aq. NH₄OH solution. The organic extract was washed with brine solution, dried (Na₂SO₄), filtered and rotary evaporated to give 329 mg (94%) of the free base of the titled compound as a light yellow oil. The sample was converted to the fumaric acid salt and precipitated from cold 2-propanol (minimum amount) and acetonitrile to give (S)-2-phenoxy-3-(piperidin-3-ylmethoxy)-pyridine fumaric acid as a white solid.

### Example 95

### Synthesis of (S)-6-methyl-2-phenoxy-3-(piperidin-3-ylmethoxy)-pyridine fumaric acid

A solution of (S)-3-(6-methyl-2-phenoxy-pyridin-3-yloxymethyl)-piperidine-1-carboxylic acid tert-butyl ester (564 mg, 1.42 mmol) in 10 mL of CH₂Cl₂ was treated with 2 mL of trifluoroacetic acid. The solution was stirred at room temperature under N₂ for 3 hours, rotary evaporated, then partitioned between CH₂Cl₂ and 10% aq. NH₄OH solution. The organic extract was washed with brine solution, dried (Na₂SO₄), filtered and rotary evaporated to give 392 mg (93%) of the free base of the titled compound as a light yellow oil. The sample was converted to the fumaric acid salt and precipitated from cold 2-propanol (minimum amount) and acetonitrile to give (S)-6-methyl-2-phenoxy-3-(piperidin-3-ylmethoxy)-pyridine fumaric acid as a white solid.

### Example 99

### Synthesis of (S)-3-(2-Benzyloxy-phenoxymethyl)-piperidine hydrochloride

A solution of (S)-3-(2-benzyloxy-phenoxy-methyl)piperidine-1-carboxylic acid tert-butyl ester (Preparation 25, 0.707 g, 1.778 mmol) in 100 mL of CH₂Cl₂ was treated with 10 mL of trifluoroacetic acid. The solution was stirred at room temperature under N₂ for 2 hours, rotary evaporated then partitioned between CHCl₃ and 10% aqueous NH₄OH solution. The organic extract was washed with brine solution, dried (MgSO₄), filtered and rotary evaporated to give 0.513 g (97%) of the free base of the titled compound as a light yellow oil. The sample was converted to the HCl acid salt and precipitated from diethyl ether to give (S)-3-(2-benzyloxy-phenoxymethyl)-piperidine hydrochloride as white solid.

### Example 101

### Synthesis of (5)-3-(2-cyclohexyloxy-phenoxymethyl)-piperidine hydrochloride

A solution of (S)-3-(2-cyclohexyloxy-phenoxymethyl)-piperidine-1-carboxylic acid tert-butyl ester (Preparation 28, 0.75 g, 1.92 mmol) in 100 mL of CH₂Cl₂ was treated with 10 mL of trifluoroacetic acid. The solution was stirred at room temperature under N₂ for 2 hours, rotary evaporated then partitioned between CHCl₃ and 10% aqueous NH₄OH solution. The organic extract was washed with brine solution, dried (MgSO₄), filtered and rotary evaporated to give 0.52 g (94%) of the free base of the titled compound as a light yellow oil. The sample was converted to the HCl acid salt and precipitated from diethyl ether to give (S)-3-(2-cyclohexyloxy-phenoxymethyl)-piperidine hydrochloride as white solid.

### Example 106

### Synthesis of (S)-3-[(2-ethoxy-phenoxy)-(R,S)-phenyl-methyl]piperidine fumaric acid

A solution of (S)-3-[(2-ethoxy-phenoxy)-(R,S)-phenyl-methyl]-piperidine-1-carboxylic acid tert-butyl ester (Preparation 29, 0.99 g, 2.42 mmol) in 100 mL of CH₂Cl₂ was treated with 10 mL of trifluoroacetic acid. The solution was stirred at room temperature under N₂ for 2 hours, rotary evaporated then partitioned between CHCl₃ and 10% aq. NH₄OH solution. The organic extract was washed with brine solution, dried (MgSO₄), filtered and rotary evaporated to give 0.79 g (>100%) of the free base of the titled compound as yellow oil. The sample was converted to the fumaric acid salt and precipitated from acetonitrile to give (S)-3-[(2-ethoxy-phenoxy)-(R,S)-phenyl-methyl]-piperidine fumarate as white solid.

The compounds of Examples 2 to 22 and 24 to 31 were prepared by adapting the procedures of Preparations 1, 2, and Example 1.

The compound of Example 23 was prepared by adapting the procedures of Preparations 1, 23, and Example 23.

The compounds of Examples 32 to 40 were prepared by adapting the procedures of Preparations 3 to 5 and Example 1.

The compound of Example 41 was prepared by adapting the procedures of Preparations 9, 10, and Example 95.

The compounds of Examples 42, 44, and 47 to 50 were prepared by adapting the procedures of Preparations 1 and 24 and Example 45.

The compounds of Examples 51 to 76, and 94 were prepared by adapting the procedures of Preparations 6, 7, and Example 93.

The compounds of Examples 78 to 83 were prepared by adapting the procedures of Preparations 6, 8, and Example 77.

The compounds of Examples 85 to 87 were prepared by adapting the procedures of Preparations 11 to 16 and Example 84.

The compounds of Examples 89 to 92 were prepared by adapting the procedures of Preparations 17 to 21 and Example 88.

The compounds of Examples 43 and 100 were prepared by adapting the procedures of Preparation 25 and Example 99.

The compounds of Examples 96 and 97 were prepared by adapting the procedures of Preparation 26 and Example 46.

The compounds of Example 98 were prepared by adapting the procedures of Preparation 25 and Example 99, wherein the TFA salt precipitated from the deprotection (i.e., BOC removal) step.

The compounds of Examples 102, 103, and 105 were prepared by adapting the procedures of Preparations 27 and 28 and Example 101.

The compound of Example 104 was prepared by using the procedure of Preparation 35 and by adapting the procedures of Preparation 28 and Example 101.

The compound of Example 107 was prepared by using the procedures of Preparations 30 and 31 and by adapting the procedure of Preparation 25. The HCl salt of Example 107 was prepared from the free base by adapting the procedure of Example 99.

The compound of Example 108 was prepared by adapting the procedures of Preparations 30, 31, and 25 and Example 99, and then dissolving the free base of the title compound in ethyl ether, adding oxalic acid, and filtering off the precipitated oxalic acid salt.

The compounds of Examples 109, 110, 111, 112, and 113 were prepared by adapting the procedures of Preparations 30, 31, and 28 and Example 101.

The compounds of Examples 114, 115, 116, and 117 were prepared by using the procedures of Preparations 36 and 37 and by adapting the procedure of Example 106.

The compound of Example 118 was prepared by using the procedures of Preparations 32 to 34, and then by adapting the procedures of Preparations 4, 28, and Example 101.

One of ordinary skill in the art may adapt the procedures of the Preparations and Examples to synthesize the invention compounds. In the adaptation of Preparation 2, for example, an appropriately ring-substituted phenol would be used in place of the 4-fluoro-2-methyl-phenol to provide the desired compounds of Examples 2 to 31, 43, and 46, wherein the substituents R^{2A}, R^{2B}, R^{3A}, R^{3B}, and R⁴ of the compounds of Examples 2 to 31 would derive from the phenol ring substituents. In the adaptation of Preparation 3, an appropriately ring-substituted 2-fluoro-benzaldehyde would be used where necessary in place of the 4-chloro-2-fluoro-benzaldehyde and an appropriately ring-substituted phenol would be used where necessary in place of the 2-fluoro-6-methoxy phenol to provide the desired compounds of Examples 32 to 40, wherein the substituents R^{2A}, R^{2B}, R^{3A}, R^{3B}, and R⁴ of the compounds of Examples 32 to 40 would derive from the phenol ring substituents and the substituents R¹, R⁶, R⁷, and R⁸ would derive from the 2-fluoro-benzaldehyde ring substituents.

The compounds of Examples 1 to 41 are fumaric acid salts of compounds of Formula (T-1) and all have (S) stereochemistry at the first chiral carbon atom, which is indicated by the symbol *. The definitions of X¹, R⁶, R^{2A}, R^{2B}, R^{3A}, R^{3B}, and R⁴ for the compounds of Examples 1 to 41 are provided below in Table 1.

TABLE 1

| | |
|---|---|
| | (T-1) |
| 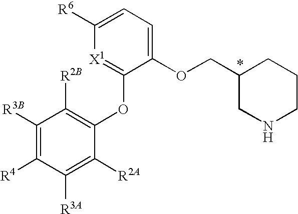 | |

| Example | | | | | | | |
|---|---|---|---|---|---|---|---|
| No. | X¹ | R⁶ | R^{2B} | R^{3B} | R⁴ | R^{3A} | R^{2A} |
| 1 | N | —CH₃ | H | H | F | H | —CH₃ |
| 2 | N | —CH₃ | —CH₃ | H | H | H | —CH₃ |
| 3 | N | —CH₃ | H | H | H | H | —OCH₃ |
| 4 | N | —CH₃ | H | H | —CH₃ | H | —OCH₃ |
| 5 | N | —CH₃ | H | H | H | H | —CH₃ |
| 6 | N | —CH₃ | H | H | —OCH₃ | H | H |
| 7 | N | —CH₃ | H | H | H | H | Cl |
| 8 | N | —CH₃ | H | H | —CH₃ | H | H |
| 9 | N | —CH₃ | H | H | H | —OCH₃ | —OCH₃ |
| 10 | N | —CH₃ | H | H | H | H | -OiPr^{a} |
| 11 | N | —CH₃ | H | H | —CH₂CH₃ | H | —OCH₃ |
| 12 | N | —CH₃ | H | H | Cl | H | —OCH₃ |
| 13 | N | —CH₃ | H | H | Cl | H | H |
| 14 | N | —CH₃ | H | H | —CH₃ | H | Cl |
| 15 | N | —CH₃ | H | Cl | H | H | H |
| 16 | N | —CH₃ | H | —CH₃ | Cl | H | iPr |
| 17 | N | —CH₃ | H | Cl | Cl | H | H |
| 18 | N | —CH₃ | H | —(CH₂)₃— | | H | H |
| 19 | N | —CH₃ | —(CH2)₄— | | H | H | H |
| 20 | N | —CH₃ | H | H | iPr | H | H |
| 21 | N | —CH₃ | F | H | H | H | —OCH₃ |
| 22 | N | —CH₃ | H | H | Cl | H | Cl |
| 23 | N | —CH₃ | H | H | Cl | H | F |
| 24 | N | —CH₃ | H | H | F | H | Cl |
| 25 | N | —CH₃ | H | H | F | H | F |
| 26 | N | —CH₃ | H | H | —OCH₃ | H | Cl |
| 27 | N | —CH₃ | H | —CF₃ | H | H | H |
| 28 | N | —CH₃ | H | H | F | H | —OCH₃ |
| 29 | N | —CH₃ | H | F | F | H | F |
| 30 | N | —CH₃ | H | H | —OCH₃ | H | F |
| 31 | N | —CH₃ | H | F | Cl | H | H |
| 32 | C—H | —OMe | H | H | Cl | H | —OCH₃ |
| 33 | C—H | Cl | H | H | Cl | H | F |
| 34 | C—H | Cl | H | H | Cl | H | —OCH₃ |
| 35 | C—H | —CF₃ | H | H | Cl | H | —OCH₃ |
| 36 | C—H | F | H | H | Cl | H | —OCH₃ |
| 37 | C—H | —CF₃ | H | H | F | H | —OCH₃ |
| 38 | C—F | H | H | H | F | H | Cl |
| 39 | C—H | F | H | H | F | H | Cl |
| 40 | C—H | Cl | F | H | H | H | —OCH₃ |
| 41 | N | —CH₃ | H | H | F | H | H |

^{a}iPr means isopropyl.

The compounds of Examples 42, 44, 45 and 47-50 are all hydrochloride salts of compounds of Formula (T-2) and all have (S) stereochemistry at the first chiral carbon atom (*). The definitions of X¹, R⁶, R^{2A}, R^{2B}, R^{3A}, R^{3B}, and R⁴ for the compounds of Examples 42, 44, 45 and 47-50 are provided below in Table 2.

TABLE 2

| | |
|---|---|
| | (T-2) |
| 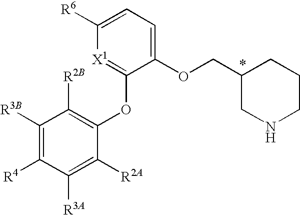 | |

| Example | | | | | | | |
|---|---|---|---|---|---|---|---|
| No. | X¹ | R⁶ | R^{2B} | R^{3B} | R⁴ | R^{3A} | R^{2A} |
| 42 | N | —CH₃ | F | H | H | H | F |
| 44 | N | —CH₃ | F | H | F | H | F |
| 45 | N | —CH₃ | F | H | Cl | H | F |
| 47 | N | —CH₃ | Cl | H | H | H | Cl |
| 48 | N | —CH₃ | Cl | H | F | H | Cl |
| 49 | N | —CH₃ | F | H | H | F | F |
| 50 | N | —CH₃ | F | H | H | Cl | F |

The compounds of Examples 43 and 46 are included with Examples 96 to 98 below.

The compounds of Examples 51 to 76 are all fumaric acid salts of compounds of Formula (T-3). The definitions of stereochemistry at the first chiral carbon atom (*) and groups R^{2B}, R^{2B}, R^{3A}, R^{3B}, R⁴ for the compounds of Examples 51 to 76 are provided below in Table 3.

TABLE 3

| | |
|---|---|
| | (T-3) |
| 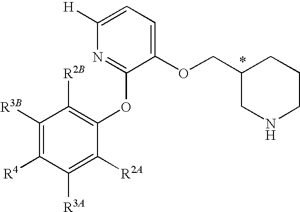 | |

| Example | | | | | | |
|---|---|---|---|---|---|---|
| No. | * | R^{2B} | R^{3B} | R⁴ | R^{3A} | R^{2A} |
| 51 | (S) | H | H | —OCH₃ | H | H |
| 52 | (S) | H | H | —CH₃ | H | H |
| 53 | (S) | H | H | F | H | H |
| 54 | (S) | H | H | F | H | F |
| 55 | (S) | H | H | Cl | H | H |
| 56 | (S) | H | H | F | H | H |
| 57 | (S) | H | H | —CN | H | H |
| 58 | (S) | H | H | —CN | —CN | H |
| 59 | (S) | H | H | H | Cl | H |
| 60 | (S) | H | H | F | F | H |
| 61 | (S) | H | H | —CN | F | H |
| 62 | (S) | H | H | —CN | H | —OCH₃ |
| 63 | (S) | H | H | F | F | H |
| 64 | (S) | H | H | Cl | Cl | H |
| 65 | (S) | H | H | —OCH₃ | —OCH₃ | H |
| 66 | (S) | H | H | F | Cl | H |
| 67 | (S) | H | H | F | —CH₃ | H |
| 68 | (S) | H | H | H | —CH₃ | H |
| 69 | (S) | H | H | Cl | F | H |
| 70 | (S) | H | H | H | —OCF₃ | H |
| 71 | (S) | H | H | F | H | Cl |
| 72 | (S) | F | H | H | H | F |
| 73 | (S) | H | H | H | H | —CH₃ |
| 74 | (S) | H | H | H | H | -OiPr^{a} |
| 75 | (S) | H | H | H | H | -iPr^{b} |
| 76 | (S) | H | —CH₃ | H | H | Cl |
| 77 | (S) | H | H | F | H | Cl |
| 78 | (S) | H | H | F | H | Cl |
| 79 | (S) | H | H | F | H | Cl |
| 80 | (S) | H | H | F | H | Cl |
| 81 | (S) | H | H | F | H | Cl |
| 82 | (S) | H | H | F | H | Cl |
| 83 | (S) | H | H | F | H | Cl |

^{a}-OiPr means isopropyloxy;
^{b}-iPr means isopropyl

The compounds of Examples 77 to 83 are all fumaric acid salts of compounds of Formula (T-4). The definitions of stereochemistry at the first chiral carbon atom (*) and X² for the compounds of Examples 77 to 83 are provided below in Table 4.

TABLE 4

| | |
|---|---|
| | (T-4) |
| 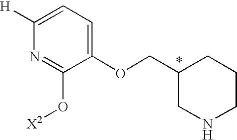 | |

| Example | | |
|---|---|---|
| No. | * | X² |
| 77 | (S) | —CH₂-phenyl |
| 78 | (S) | —CH(CH₃)CH₂CH₃ |
| 79 | (S) | —CH₂CH₃ |
| 80 | (S) | —CH(CH₃)CH₃ |
| 81 | (S) | cyclohexyl |
| 82 | (S) | —CH₂CH₂-phenyl |
| 83 | (S) | —CH₂CH₂CH₂-phenyl |

The compounds of Examples 84 to 92 are all fumaric acid salts of compounds of Formula (T-5). The definitions of stereochemistry at the first chiral carbon (*), stereochemistry at the second chiral carbon atom, which is identified by the symbol ̂, and the groups X², and R^{5A} for the compounds of Examples 84 to 92 are provided below in Table 5.

TABLE 5

| | |
|---|---|
| | (T-5) |
| 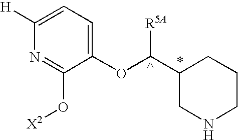 | |

| Example | | | | |
|---|---|---|---|---|
| No. | * | \hat{ } | X² | R^{5A} |
| 84 | (S) | (S) | 4-fluorophenyl | —CH₂CH₃ |
| 85 | (S) | (S) | phenyl | —CH₂CH₃ |
| 86 | (S) | (S) | —CH₂CH₃ | —CH₂CH₃ |
| 87 | (S) | (S) | —CH(CH₃)CH₂CH₃ | —CH₂CH₃ |
| 88 | (S) | stereoisomer (A)^{a} | 4-fluorophenyl | phenyl |
| 89 | (S) | stereoisomer (B)^{a} | 4-fluorophenyl | phenyl |
| 90 | (S) | stereoisomer (C)^{b} | —CH₂CH₃ | phenyl |
| 91 | (S) | stereoisomer (D)^{b} | —CH₂CH₃ | phenyl |
| 92 | (S) | stereoisomer (E)^{c} | —CH(CH₃)CH₂CH₃ | phenyl |

^{a}stereoisomer (A) and stereoisomer (B) refer to the separated enantiomers of the compounds of Examples 88 and 89, respectively, wherein the stereochemistry at their second chiral carbons indicated with the symbol \hat{ } is unassigned and the compounds of Examples 88 and 89 are epimeric to each other at the second chiral carbons;
^{b}stereoisomer (C) and stereoisomer (D) refer to the separated enantiomers of the compounds of Examples 90 and 91, respectively, wherein the stereochemistry at their second chiral carbons indicated with the symbol \hat{ } is unassigned and the compounds of Examples 90 and 91 are epimeric to each other at the second chiral carbons;
^{c}stereoisomer (E) refers to one of the two possible stereoisomers of the compound of Example 92, wherein the stereochemistry at its second chiral carbon indicated with the symbol \hat{ } is unassigned.

The compounds of Examples 93 to 95 are all fumaric acid salts of compounds of Formula (T-6). The definitions of stereochemistry at the first chiral carbon atom (*) and groups R⁶ and X² for the compounds of Examples 93 to 95 are provided below in Table 6.

TABLE 6

| | |
|---|---|
| | (T-6) |
| 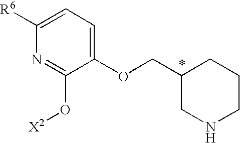 | |

| Example | | | |
|---|---|---|---|
| No. | * | R⁶ | X² |
| 93 | (S) | H | phenyl |
| 94 | (R) | H | phenyl |
| 95 | (S) | —CH₃ | phenyl |

The definitions of stereochemistry at the first chiral carbon atom (*) and groups R¹, R^{2A}, R^{2B}, R^{3A}, R^{3B}, R⁴, R⁶, and R⁸ and the acid component of the salts of the compounds of Examples 43, 46, and 96 to 98 are provided below in Table 7.

TABLE 7

| | |
|---|---|
| | (T-7) |
| 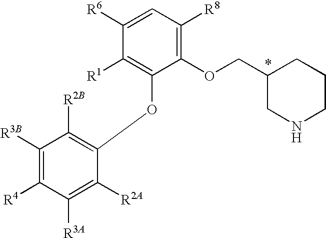 | |

| Ex- | | | | | | | | | | |
|---|---|---|---|---|---|---|---|---|---|---|
| ample | | | | | | | | | | |
| No. | * | R^{2B} | R^{3B} | R⁴ | R^{3A} | R^{2A} | R¹ | R⁶ | R⁸ | Acid |
| 43 | S | H | H | H | H | H | H | H | H | HCl |
| 46 | S | H | H | H | H | H | H | F | H | fumaric |
| | | | | | | | | | | acid |
| 96 | S | H | H | F | H | H | H | H | F | fumaric |
| | | | | | | | | | | acid |
| 97 | S | H | F | F | H | H | H | H | F | fumaric |
| | | | | | | | | | | acid |
| 98 | S | H | H | F | H | H | F | H | H | CF₃CO₂H |

The definitions of stereochemistry at the first chiral carbon atom (*) and groups R¹, R⁶, R⁸ and X² and the acid component of the salts of the compounds of Examples 99 to 105 are provided below in Table 8.

TABLE 8

| | |
|---|---|
| | (T-8) |
| 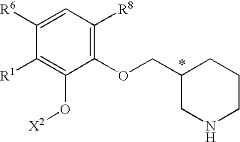 | |

| Example | | | | | | |
|---|---|---|---|---|---|---|
| No. | * | R¹ | R⁶ | R⁸ | X² | Acid |
| 99 | (S) | H | H | H | —CH₂phenyl | HCl |
| 100 | (S) | H | H | H | —CH₂CH₃ | fumaric acid |
| 101 | (S) | H | H | H | cyclohexyl | HCl |
| 102 | (S) | H | H | H | —CH₂CH(CH₃)₂ | HCl |
| 103 | (S) | H | H | F | —CH₃ | fumaric acid |
| 104 | (S) | H | H | F | —CH₂CH(CH₃)₂ | fumaric acid |
| 105 | (S) | H | H | F | —CH₂CH₃ | fumaric acid |

For the compounds of Examples 106 to 117, the definitions of stereochemistry at the first (*) and second (̂) chiral carbon atoms and the groups R¹, R^{5A}, R⁶, R⁷, R⁸ and X² and the acid component of the salts are provided below in Table 9.

TABLE 9

| | |
|---|---|
| | (T-9) |
| 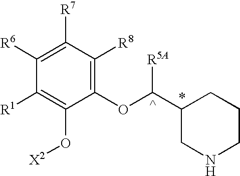 | |

| Example | | | | | | | | | |
|---|---|---|---|---|---|---|---|---|---|
| No. | * | \hat{ } | R¹ | R⁶ | R⁷ | R⁸ | R^{5A} | X² | Acid |
| 106 | (S) | (R/S)^{a} | H | H | H | H | phenyl | —CH₂CH₃ | HCl |
| 107 | (S) | (S) | H | H | H | H | —CH₃ | —CH₂CH₃ | none^{b} |
| 108 | (S) | (S) | H | H | H | H | —CH₃ | —CH₂phenyl | oxalic |
| | | | | | | | | | acid |
| 109 | (S) | (S) | H | H | H | H | —CH₃ | —CH₂CH(CH₃)₂ | none |
| 110 | (S) | (S) | H | H | H | H | —CH₃ | —CH₂cyclobutyl | oxalic |
| | | | | | | | | | acid |
| 111 | (S) | (S) | H | H | H | H | —CH₃ | cyclohexyl | oxalic |
| | | | | | | | | | acid |
| 112 | (S) | (S) | H | H | H | H | —CH₃ | —(CH₂)₂CH(CH₃)₂ | HCl |
| 113 | (S) | (S) | H | H | H | H | —CH₃ | —(CH₂)₂OCH₃ | HCl |
| 114 | (S) | (R) | H | H | H | H | pyridin- | —CH₂CH₃ | fumaric |
| | | | | | | | 2-yl | | acid |
| 115 | (S) | (R) | H | H | H | H | pyridin- | —CH₃ | fumaric |
| | | | | | | | 2-yl | | acid |
| 116 | (S) | (R) | H | H | H | H | pyridin- | —CF₃ | fumaric |
| | | | | | | | 2-yl | | acid |
| 117 | (S) | (R) | H | H | F | H | pyridin- | —CH₃ | fumaric |
| | | | | | | | 2-yl | | acid |

^{a}(R/S) means a 50:50 mixture of epimers at \hat{ };
^{b}free base

The definitions of stereochemistry at the first (*) and second (̂) chiral carbon atoms and groups R¹, R^{5A}, R^{5B}, R⁶, R⁷, R⁸ and the acid component of the salt of the compound of Example 118 is provided below in Table 10.

TABLE 10

| | |
|---|---|
| | (T-10) |
| 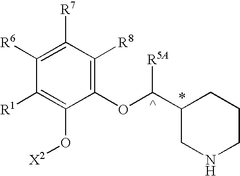 | |

| Example | | | | | | | | | |
|---|---|---|---|---|---|---|---|---|---|
| No. | * | R¹ | R⁶ | R⁷ | R⁸ | R^{5A} | R^{5B} | X² | Acid |
| 118 | (S) | H | H | H | H | —CH₃ | —CH₃ | —(CH₂)₂OCH₃ | Fumaric acid |

Another embodiment is a compound of Formula (T-1), (T-2), (T-3), (T-4), (T-5), (T-6), (T-7), (T-8), (T-9), or (T-10), or a pharmaceutically acceptable acid addition salt thereof, wherein *, X¹, X², R¹, R^{2A}, R^{2B}, R^{3A}, R^{3B}, R⁴, R^{5A}, R^{5B}, R⁶, R⁷, R^{7A}, R^{7B}, R^{7C}, and R⁸ are as defined for Formula (I).

The names of the invention compounds or salts of Examples 1 to 118 are provided in Table 11. In Table 11, “Ex. No.” means Example Number.

TABLE 11

| Ex. | |
|---|---|
| No. | Compound Name |
| 1 | (S)-2-(4-fluoro-2-methyl-phenoxy)-6-methyl-3-(piperidin-3-ylmethoxy)-pyridine |
| | fumaric acid |
| 2 | (S)-2-(2,6-dimethyl-phenoxy)-6-methyl-3-(piperidin-3-ylmethoxy)-pyridine fumaric |
| | acid |
| 3 | (S)-2-(2-methoxy-phenoxy)-6-methyl-3-(piperidin-3-ylmethoxy)-pyridine fumaric |
| | acid |
| 4 | (S)-2-(2-methoxy-4-methyl-phenoxy)-6-methyl-3-(piperidin-3-ylmethoxy)-pyridine |
| | fumaric acid |
| 5 | (S)-2-(2-methyl-phenoxy)-6-methyl-3-(piperidin-3-ylmethoxy)-pyridine fumaric acid |
| 6 | (S)-2-(4-methoxy-phenoxy)-6-methyl-3-(piperidin-3-ylmethoxy)-pyridine fumaric |
| | acid |
| 7 | (S)-2-(2-chloro-phenoxy)-6-methyl-3-(piperidin-3-ylmethoxy)-pyridine fumaric acid |
| 8 | (S)-2-(4-methyl-phenoxy)-6-methyl-3-(piperidin-3-ylmethoxy)-pyridine fumaric acid |
| 9 | (S)-2-(2,3-dimethoxy-phenoxy)-6-methyl-3-(piperidin-3-ylmethoxy)-pyridine fumaric |
| | acid |
| 10 | (S)-2-[2-(1-methyl-ethoxy)-phenoxy]-6-methyl-3-(piperidin-3-ylmethoxy)-pyridine |
| | fumaric acid |
| 11 | (S)-2-(4-ethyl-2-methoxy-phenoxy)-6-methyl-3-(piperidin-3-ylmethoxy)-pyridine |
| | fumaric acid |
| 12 | (S)-2-(4-chloro-2-methoxy-phenoxy)-6-methyl-3-(piperidin-3-ylmethoxy)-pyridine |
| | fumaric acid |
| 13 | (S)-2-(4-chloro-phenoxy)-6-methyl-3-(piperidin-3-ylmethoxy)-pyridine fumaric acid |
| 14 | (S)-2-(2-chloro-4-methyl-phenoxy)-6-methyl-3-(piperidin-3-ylmethoxy)-pyridine |
| | fumaric acid |
| 15 | (S)-2-(3-chloro-phenoxy)-6-methyl-3-(piperidin-3-ylmethoxy)-pyridine fumaric acid |
| 16 | (S)-2-[4-chloro-5-methyl-2-(1-methyl-ethyl)-phenoxy]-6-methyl-3-(piperidin-3- |
| | ylmethoxy)-pyridine fumaric acid |
| 17 | (S)-2-(3,4-dichloro-phenoxy)-6-methyl-3-(piperidin-3-ylmethoxy)-pyridine fumaric |
| | acid |
| 18 | (S)-2-(2,3-dihydro-1H-inden-5-yloxy)-6-methyl-3-(piperidin-3-ylmethoxy)pyridine |
| | fumaric acid |
| 19 | (S)-6-methyl-3-(piperidin-3-ylmethoxy)-2-(5,6,7,8-tetrahydronaphthalen-1-yloxy)- |
| | pyridine fumaric acid |
| 20 | (S)-2-[4-(1-methyl-ethyl)-phenoxy]-6-methyl-3-(piperidin-3-ylmethoxy)-pyridine |
| | fumaric acid |
| 21 | (S)-2-(2-fluoro-6-methoxy-phenoxy)-6-methyl-3-(piperidin-3-ylmethoxy)-pyridine |
| | fumaric acid |
| 22 | (S)-2-(2,4-dichloro-phenoxy)-6-methyl-3-(piperidin-3-ylmethoxy)-pyridine fumaric |
| | acid |
| 23 | (S)-2-(4-chloro-2-fluoro-phenoxy)-6-methyl-3-(piperidin-3-ylmethoxy)-pyridine |
| | fumaric acid |
| 24 | (S)-2-(2-chloro-4-fluoro-phenoxy)-6-methyl-3-(piperidin-3-ylmethoxy)-pyridine |
| | fumaric acid |
| 25 | (S)-2-(2,4-difluoro-phenoxy)-6-methyl-3-(piperidin-3-ylmethoxy)-pyridine fumaric |
| | acid |
| 26 | (S)-2-(2-chloro-4-methoxy-phenoxy)-6-methyl-3-(piperidin-3-ylmethoxy)-pyridine |
| | fumaric acid |
| 27 | (S)-6-methyl-2-(3-trifluoromethy-phenoxy)-3-(piperidin-3-ylmethoxy)-pyridine |
| | fumaric acid |
| 28 | (S)-2-(4-fluoro-2-methoxy-phenoxy)-6-methyl-3-(piperidin-3-ylmethoxy)-pyridine |
| | fumaric acid |
| 29 | (S)-6-methyl-2-(2,4,5-trifluoro-phenoxy)-3-(piperidin-3-ylmethoxy)-pyridine fumaric |
| | acid |
| 30 | (S)-2-(2-fluoro-4-methoxy-phenoxy)-6-methyl-3-(piperidin-3-ylmethoxy)-pyridine |
| | fumaric acid |
| 31 | (S)-2-(4-chloro-3-fluoro-phenoxy)-6-methyl-3-(piperidin-3-ylmethoxy)-pyridine |
| | fumaric acid |
| 32 | (S)-3-[2-(4-chloro-2-methoxy-phenoxy)-4-methoxy-phenoxymethyl]-piperidine |
| | fumaric acid |
| 33 | (S)-3-[4-chloro-2-(4-chloro-2-fluoro-phenoxy)-phenoxymethyl]-piperidine fumaric |
| | acid |
| 34 | (S)-3-[4-chloro-2-(4-chloro-2-methoxy-phenoxy)-phenoxymethyl]-piperidine fumaric |
| | acid |
| 35 | (S)-3-[2-(4-chloro-2-methoxy-phenoxy)-4-trifluoromethyl-phenoxymethyl]-piperidine |
| | fumaric acid |
| 36 | (S)-3-[2-(4-chloro-2-methoxy-phenoxy)-4-fluoro-phenoxymethyl]-piperidine fumaric |
| | acid |
| 37 | (S)-3-[2-(4-fluoro-2-methoxy-phenoxy)-4-trifluoromethyl-phenoxymethyl]-piperidine |
| | fumaric acid |
| 38 | (S)-3-[2-(2-chloro-4-fluoro-phenoxy)-3-fluoro-phenoxymethyl]-piperidine fumaric |
| | acid |
| 39 | (S)-3-[2-(2-chloro-4-fluoro-phenoxy)-4-fluoro-phenoxymethyl]-piperidine fumaric |
| | acid |
| 40 | (S)-3-[4-chloro-2-(2-fluoro-6-methoxy-phenoxy)-phenoxymethyl]-piperidine fumaric |
| | acid |
| 41 | (S)-2-(4-fluoro-phenoxy)-6-methyl-3-(piperidin-3-ylmethoxy)-pyridine fumaric acid |
| 42 | (S)-2-(2,6-difluoro-phenoxy)-6-methyl-3-(piperidin-3-ylmethoxy)-pyridine |
| | hydrochloride |
| 43 | (S)-3-(2-phenoxy-phenoxymethyl)-piperidine hydrochloride |
| 44 | (S)-6-methyl-2-(2,4,6-trifluoro-phenoxy)-3-(piperidin-3-ylmethoxy)-pyridine |
| | hydrochloride |
| 45 | (S)-2-(4-chloro-2,6-difluoro-phenoxy)-6-methyl-3-(piperidin-3-ylmethoxy)-pyridine |
| | hydrochloride |
| 46 | (S)-3-(4-fluoro-2-phenoxy-phenoxymethyl)-piperidine fumaric acid |
| 47 | (S)-2-(2,6-dichloro-phenoxy)-6-methyl-3-(piperidin-3-ylmethoxy)-pyridine |
| | hydrochloride |
| 48 | (S)-2-(2,6-dichloro-4-fluoro-phenoxy)-6-methyl-3-(piperidin-3-ylmethoxy)-pyridine |
| | hydrochloride |
| 49 | (S)-6-methyl-3-(piperidin-3-ylmethoxy)-2-(2,3,6-trifluoro-phenoxy)-pyridine |
| | hydrochloride |
| 50 | (S)-2-(3-chloro-2,6-difluoro-phenoxy)-6-methyl-3-(piperidin-3-ylmethoxy)-pyridine |
| | hydrochloride |
| 51 | (S)-2-(4-methoxy-phenoxy)-3-(piperidin-3-ylmethoxy)-pyridine fumaric acid |
| 52 | (S)-2-(4-methyl-phenoxy)-3-(piperidin-3-ylmethoxy)-pyridine fumaric acid |
| 53 | (S)-2-(4-fluoro-phenoxy)-3-(piperidin-3-ylmethoxy)-pyridine fumaric acid |
| 54 | (S)-2-(2,4-difluoro-phenoxy)-3-(piperidin-3-ylmethoxy)-pyridine fumaric acid |
| 55 | (S)-2-(4-chloro-phenoxy)-3-(piperidin-3-ylmethoxy)-pyridine fumaric acid |
| 56 | (R)-2-(4-fluoro-phenoxy)-3-(piperidin-3-ylmethoxy)-pyridine fumaric acid |
| 57 | (S)-4-[3-(piperidin-3-ylmethoxy)-pyridin-2-yloxy]-benzonitrile fumaric acid |
| 58 | (S)-4-[3-(piperidin-3-ylmethoxy)-pyridin-2-yloxy]-phthalonitrile fumaric acid |
| 59 | (S)-2-(3-chloro-phenoxy)-3-(piperidin-3-ylmethoxy)-pyridine fumaric acid |
| 60 | (S)-2-(3,4-difluoro-phenoxy)-3-(piperidin-3-ylmethoxy)-pyridine fumaric acid |
| 61 | (S)-2-fluoro-4-[3-(piperidin-3-ylmethoxy)-pyridin-2-yloxy]-benzonitrile fumaric acid |
| 62 | (S)-3-methoxy-4-[3-(piperidin-3-ylmethoxy)-pyridin-2-yloxy]-benzonitrile fumaric |
| | acid |
| 63 | (R)-2-(3,4-difluoro-phenoxy)-3-(piperidin-3-ylmethoxy)-pyridine fumaric acid |
| 64 | (S)-2-(3,4-dichloro-phenoxy)-3-(piperidin-3-ylmethoxy)-pyridine fumaric acid |
| 65 | (S)-2-(3,4-dimethyl-phenoxy)-3-(piperidin-3-ylmethoxy)-pyridine fumaric acid |
| 66 | (S)-2-(3-chloro-4-fluoro-phenoxy)-3-(piperidin-3-ylmethoxy)-pyridine fumaric acid |
| 67 | (S)-2-(4-fluoro-3-methyl-phenoxy)-3-(piperidin-3-ylmethoxy)-pyridine fumaric acid |
| 68 | (S)-2-(3-methyl-phenoxy)-3-(piperidin-3-ylmethoxy)-pyridine fumaric acid |
| 69 | (S)-2-(4-chloro-3-fluoro-phenoxy)-3-(piperidin-3-ylmethoxy)-pyridine fumaric acid |
| 70 | (S)-3-(piperidin-3-ylmethoxy)-2-(3-trifluoromethoxy-phenoxy)-pyridine fumaric acid |
| 71 | (S)-2-(2-chloro-4-fluoro-phenoxy)-3-(piperidin-3-ylmethoxy)-pyridine fumaric acid |
| 72 | (S)-2-(2,6-difluoro-phenoxy)-3-(piperidin-3-ylmethoxy)-pyridine fumaric acid |
| 73 | (S)-2-(2-methyl-phenoxy)-3-(piperidin-3-ylmethoxy)-pyridine fumaric acid |
| 74 | (S)-2-(2-isopropoxy-phenoxy)-3-(piperidin-3-ylmethoxy)-pyridine fumaric acid |
| 75 | (S)-2-(2-isopropyl-phenoxy)-3-(piperidin-3-ylmethoxy)-pyridine fumaric acid |
| 76 | (S)-2-(2-chloro-5-methyl-phenoxy)-3-(piperidin-3-ylmethoxy)-pyridine fumaric acid |
| 77 | (S)-2-benzyloxy-3-(piperidin-3-ylmethoxy)-pyridine fumaric acid |
| 78 | (S)-2-isobutoxy-3-(piperidin-3-ylmethoxy)-pyridine fumaric acid |
| 79 | (S)-2-ethoxy-3-(piperidin-3-ylmethoxy)-pyridine fumaric acid |
| 80 | (S)-2-isopropoxy-3-(piperidin-3-ylmethoxy)-pyridine fumaric acid |
| 81 | (S)-2-cyclohexyloxy-3-(piperidin-3-ylmethoxy)-pyridine fumaric acid |
| 82 | (S)-2-phenethyloxy-3-(piperidin-3-ylmethoxy)-pyridine fumaric acid |
| 83 | (S)-2-(3-phenyl-propoxy)-3-(piperidin-3-ylmethoxy)-pyridine fumaric acid |
| 84 | (S,S)-2-phenoxy-3-(1-piperidin-3-yl-propoxy)-pyridine fumaric acid |
| 85 | (S,S)-2-(4-fluoro-phenoxy)-3-(1-piperidin-3-yl-propoxy)-pyridine fumaric acid |
| 86 | (S,S)-2-ethoxy-3-(1-piperidin-3-yl-propoxy)-pyridine fumaric acid |
| 87 | (S,S)-2-isobutoxy-3-(1-piperidin-3-yl-propoxy)-pyridine fumaric acid |
| 88 | 2-(4-fluoro-phenoxy)-3-[((R or S)-phenyl)-((S)-piperidin-3-yl)-methoxy]-pyridine |
| | fumaric acid, stereoisomer A^{a} |
| 89 | 2-(4-fluoro-phenoxy)-3-[((R or S)-phenyl)-((S)-piperidin-3-yl)-methoxy]-pyridine |
| | fumaric acid, stereoisomer B^{a} |
| 90 | 2-ethoxy-3-[((R or S)-phenyl)-((S)-piperidin-3-yl)-methoxy]-pyridine fumaric acid, |
| | stereoisomer A^{b} |
| 91 | 2-ethoxy-3-[((R or S)-phenyl)-((S)-piperidin-3-yl)-methoxy]-pyridine fumaric acid, |
| | stereoisomer B^{b} |
| 92 | 2-isobutoxy-3-[((R or S)-phenyl)-((S)-piperidin-3-yl)-methoxy]-pyridine fumaric acid, |
| | stereoisomer A^{c} |
| 93 | (S)-2-phenoxy-3-(piperidin-3-ylmethoxy)-pyridine fumaric acid |
| 94 | (R)-2-phenoxy-3-(piperidin-3-ylmethoxy)-pyridine fumaric acid |
| 95 | (S)-6-methyl-2-phenoxy-3-(piperidin-3-ylmethoxy)-pyridine fumaric acid |
| 96 | (S)-3-[2-fluoro-6-(4-fluoro-phenoxy)-phenoxymethyl]-piperidine fumaric acid |
| 97 | (S)-3-[2-(3,4-difluoro-phenoxy)-6-fluoro-phenoxymethyl]-piperidine fumaric acid |
| 98 | (S)-3-[3-fluoro-2-(4-fluoro-phenoxy)-phenoxymethyl]-piperidine trifluoroacetic acid |
| 99 | (S)-3-(2-benzyloxy-phenoxymethyl)-piperidine hydrochloride |
| 100 | (S)-3-(2-ethoxy-phenoxymethyl)-piperidine fumaric acid |
| 101 | (S)-3-(2-cyclohexyloxy-phenoxymethyl)-piperidine hydrochloride |
| 102 | 3-(2-isobutoxy-phenoxymethyl)-piperidine hydrochloride |
| 103 | (S)-3-(2-fluoro-6-methoxy-phenoxymethyl)-piperidine fumaric acid |
| 104 | (S)-3-(2-fluoro-6-isobutoxy-phenoxymethyl)-piperidine fumaric acid |
| 105 | (S)-3-(2-ethoxy-6-fluoro-phenoxymethyl)-piperidine fumaric acid |
| 106 | (S)-3-[(2-ethoxy-phenoxy)-(R,S)-phenyl-methyl]-piperidine fumaric acid |
| 107 | (S)-3-[(S)-1-(2-ethoxy-phenoxy)-ethyl]-piperidine |
| 108 | (S)-3-[(S)-1-(2-benzyloxy-phenoxy)-ethyl]-piperidine oxalic acid |
| 109 | (S)-3-[(S)-1-(2-isobutoxy-phenoxy)-ethyl]-piperidine |
| 110 | (S)-3-[(S)-1-(2-cyclobutylmethoxy-phenoxy)-ethyl]-piperidine oxalic acid |
| 111 | (S)-3-[(S)-1-(2-cyclohexyloxy-phenoxy)-ethyl]-piperidine oxalic acid |
| 112 | (S)-3-{(S)-1-[2-(3-methyl-butoxy)-phenoxy]-ethyl}-piperidine oxalic acid |
| 113 | (S)-3-{(S)-1-[2-(2-methoxy-ethoxy)-phenoxy]-ethyl}-piperidine hydrochloride |
| 114 | 2-[{(R)-2-ethoxy-phenoxy}-(S)-piperidin-3-yl-methyl]-pyridine fumaric acid |
| 115 | 2-[{(R)-2-fluoro-6-methoxy-phenoxy}-(S)-piperidin-3-yl-methyl]-pyridine fumaric |
| | acid |
| 116 | 2-[(S)-piperidin-3-yl-{(R)-2-trifluoromethoxy-phenoxy}-methyl]-pyridine fumaric acid |
| 117 | 2-[{(R)-5-fluoro-2-methoxy-phenoxy}-(S)-piperidin-3-yl-methyl]-pyridine fumaric |
| | acid |
| 118 | (S)-3-{1-[2-(2-methoxy-ethoxy)-phenoxy]-1-methyl-ethyl}-piperidine fumaric acid |

^{a}The compound of Example 88 is predominantly one stereoisomer, but it has not been determined whether that stereoisomer is 2-(4-fluoro-phenoxy)-3-[((S)-phenyl)-((S)-piperidin-3-yl)-methoxy]-pyridine fumaric acid or 2-(4-fluoro-phenoxy)-3-[((R)-phenyl)-((S)-piperidin-3-yl)-methoxy]-pyridine fumaric acid; the compound of Example 89 is predominantly the other stereoisomer.
^{b}The compound of Example 90 is predominantly one stereoisomer, but it has not been determined whether that stereoisomer is 2-ethoxy-3-[((S)-phenyl)-((S)-piperidin-3-yl)-methoxy]-pyridine fumaric acid or 2-ethoxy-3-[((R)-phenyl)-((S)-piperidin-3-yl)-methoxy]-pyridine fumaric acid; the compound of Example 91 is predominantly the other stereoisomer.
^{c}The compound of Example 92 is predominantly one stereoisomer, but it has not been determined whether that stereoisomer is 2-isobutoxy-3-[((S)-phenyl)-((S)-piperidin-3-yl)-methoxy]-pyridine fumaric acid or 2-isobutoxy-3-[((R)-phenyl)-((S)-piperidin-3-yl)-methoxy]-pyridine fumaric acid.

Physical characterization data for the compounds of Examples 1 to 118 are provided below in Table 12. In Table 12, the molecular weights (Mol. Wt.) of the free base forms of the compounds, not the molecular weights of the salts, are given.

TABLE 12

| Ex. | |
|---|---|
| No. | Physical Characterizing Data |
| 1 | 1H NMR (400 MHz, dimethylsulfoxide deuterium-6 (DMSO-d6)) delta (δ) parts per |
| | million (ppm) 1.19-1.34 (multiplet (m), J = 12.14, 12.14, 11.99, 3.51 Hz, 1H) |
| | 1.49-1.65 (m, 1H) 1.68-1.84 (m, 2H) 2.07-2.11 (m, 3H) 2.15-2.20 (m, 3H) |
| | 2.16-2.20 (m, 3H) 2.56-2.73 (m, 2H) 3.14 (doublet (d), J = 12.48 Hz, 1H) 3.26 (d, |
| | J = 10.53 Hz, 2H) 3.87-3.94 (m, 1H) 3.95-4.02 (m, 1H) 6.42 (singlet (s), 4H) |
| | 6.90 (d, J = 8.58 Hz, 1H) 6.98-7.02 (m, 2H) 7.13-7.16 (m, 2H) 7.39 (d, J = 7.80 |
| | Hertz (Hz), 1H) |
| | Mol. Wt. 330.4007; MS [M + 1] m/z 331.2 |
| 2 | 1H NMR (400 MHz, DMSO-d6) δ ppm 1.26-1.40 (m, 1H) 1.53-1.68 (m, 1H) |
| | 1.70-1.92 (m, 2H) 2.02 (s, 6H) 2.12 (s, 3H) 2.15-2.25 (m, 1H) 2.63-2.76 (m, |
| | 2H) 3.16 (d, J = 12.48 Hz, 1H) 3.33 (doublet of doublets (dd), J = 11.89, 2.53 Hz, 2H) |
| | 3.33 (none, 5H) 3.90-4.08 (m, 2H) 3.99-4.09 (m, 1H) 6.42 (s, 1H) 6.83 (d, |
| | J = 8.19 Hz, 1H) 6.99-7.13 (m, 3H) 7.00-7.12 (m, 3H) 7.36 (d, J = 7.80 Hz, 1H) |
| | Mol. Wt. 326.4374; MS [M + 1] m/z 327.2 |
| 3 | 1H NMR (400 MHz, DMSO-d6) δ ppm 1.18-1.36 (m, 1H) 1.49-1.69 (m, 1H) |
| | 1.67-1.88 (m, 2H) 2.14 (s, 3H) 2.55-2.77 (m, 2H) 3.15 (d, J = 12.21 Hz, 1H) |
| | 3.27 (dd, J = 12.09, 2.81 Hz, 1H) 3.68 (s, 3H) 3.83-3.95 (m, 1H) 3.94-4.04 (m, 1H) |
| | 6.42 (s, 2H) 6.85 (d, J = 8.30 Hz, 1H) 6.89-6.99 (m, 1H) 6.98-7.07 (m, 1H) |
| | 7.08-7.24 (m, 2H) 7.34 (d, J = 7.82 Hz, 1H) |
| | Mol. Wt. 328.4096; MS [M + 1] m/z 329.2 |
| 4 | 1H NMR (400 MHz, DMSO-d6) δ ppm 1.18-1.35 (m, 1H) 1.50-1.66 (m, 1H) |
| | 1.69-1.88 (m, 2H) 2.10-2.19 (m, 4H) 2.32 (s, 3H) 2.56-2.75 (m, 2H) 3.14 (d, |
| | J = 12.09 Hz, 1H) 3.66 (s, 3H) 3.86-3.93 (m, 1H) 3.95-4.01 (m, 1H) 6.39 (s, 2H) |
| | 6.66-6.75 (m, 1H) 6.80 (d, J = 7.80 Hz, 1H) 6.83-6.94 (m, 2H) 7.29 (d, |
| | J = 7.80 Hz, 1H) |
| | Mol. Wt. 342.4364; MS [M + 1] m/z 343.2 |
| 5 | 1H NMR (400 MHz, DMSO-d6) δ ppm 1.15-1.32 (m, 1H) 1.47-1.65 (m, 1H) |
| | 1.67-1.84 (m, 2H) 2.11 (s, 3H) 2.19 (s, 3H) 2.53-2.71 (m, 2H) 3.13 (d, |
| | J = 12.48 Hz, 1H) 3.22 (dd, J = 12.48, 3.12 Hz, 1H) 3.84-3.94 (m, 1H) |
| | 3.94-4.03 (m, 1H) 6.42 (s, 2H) 6.86-6.96 (m, 2H) 7.03-7.12 (m, 1H) 7.13-7.22 (m, 1H) |
| | 7.27 (d, J = 6.63 Hz, 1H) 7.39 (d, J = 7.80 Hz, 1H) |
| | Mol. Wt. 312.4106; MS [M + 1] m/z 313.2 |
| 6 | 1H NMR (400 MHz, DMSO-d6) δ ppm 1.18-1.34 (m, 1H) 1.50-1.67 (m, 1H) |
| | 1.69-1.85 (m, 2H) 2.21 (s, 3H) 2.55-2.74 (m, 2H) 3.15 (d, J = 12.09 Hz, 1H) |
| | 3.26 (dd, J = 12.48, 3.12 Hz, 1H) 3.76 (s, 3H) 3.85-3.93 (m, 1H) 3.94-4.01 (m, 1H) |
| | 6.43 (s, 2H) 6.88-6.96 (m, 3H) 6.99 (d, 1H) |
| | Mol. Wt. 328.4096; MS [M + 1] m/z 329.1 |
| 7 | 1H NMR (400 MHz, DMSO-d6) δ ppm 1.18-1.31 (m, 1H) 1.48-1.63 (m, 1H) |
| | 1.68-1.83 (m, 2H) 2.09 (s, 1H) 2.19 (s, 3H) 2.53-2.64 (m, 2H) 2.64-2.72 (m, |
| | 1H) 3.12 (d, J = 11.31 Hz, 1H) 3.24 (d, J = 12.87 Hz, 1H) 3.87-3.95 (m, 1H) |
| | 3.97-4.04 (m, 1H) 6.42 (s, 2H) 7.14-7.19 (m, 1H) 7.23 (triplet of doublets (td), J = 7.80, |
| | 1.56 Hz, 1H) 7.36 (td, J = 7.80, 1.56 Hz, 1H) 7.44 (d, J = 8.19 Hz, 1H) |
| | Mol. Wt. 332.8289; MS [M + 1] m/z 333.1 |
| 8 | 1H NMR (400 MHz, DMSO-d6) δ ppm 1.47-1.62 (m, 1H) 1.66-1.79 (m, 2H) |
| | 2.02-2.14 (m, 1H) 2.21 (s, 3H) 2.29 (s, 3H) 2.55 (triplet (t), 1H) 2.64 (td, |
| | J = 12.18, 3.31 Hz, 1H) 3.12 (d, J = 12.09 Hz, 1H) 3.20 (dd, J = 12.09, 3.12 Hz, 3H) |
| | 3.83-3.91 (m, 1H) 3.92-3.98 (m, 1H) 6.42 (s, 2H) 6.90 (doublet of doublets of |
| | doublets (ddd), J = 8.97, 2.73, 2.34 Hz, 2H) 6.94 (d, J = 7.80 Hz, 1H) 7.16 (d, |
| | J = 8.58 Hz, 2H) 7.39 (d, J = 7.80 Hz, 1H) |
| | Mol. Wt. 312.4106; MS [M + 1] m/z 313.1 |
| 9 | 1H NMR (400 MHz, DMSO-d6) δ ppm 1.18-1.35 (m, 1H) 1.49-1.64 (m, 1H) |
| | 1.67-1.85 (m, 2H) 2.06-2.17 (m, 1H) 2.18 (s, 3H) 2.55-2.73 (m, 2H) 3.14 (d, |
| | J = 12.48 Hz, 1H) 3.21-3.28 (m, J = 12.87 Hz, 1H) 3.58 (s, 3H) 3.83 (s, 3H) |
| | 3.87-3.94 (m, 1H) 3.95-4.05 (m, 1H) 6.42 (s, 2H) 6.66 (dd, J = 8.19, 1.56 Hz, 1H) |
| | 6.87-6.92 (m, J = 8.19 Hz, 2H) 7.04 (t, J = 8.19 Hz, 1H) 7.37 (d, J = 7.80 Hz, 1H) |
| | Mol. Wt. 358.4354; MS [M + 1] m/z 360.1 |
| 10 | 1H NMR (400 MHz, DMSO-d6) δ ppm 1.01 (dd, J = 6.04, 2.92 Hz, 6H) |
| | 1.20-1.34 (m, 1H) 1.52-1.66 (m, 1H) 1.69-1.86 (m, 2H) 2.14 (s, 3H) 2.55-2.74 (m, 2H) |
| | 3.14 (d, J = 11.31 Hz, 1H) 3.22-3.29 (m, J = 11.70 Hz, 1H) 3.84-3.94 (m, 1H) |
| | 3.94-4.02 (m, 1H) 4.41-4.55 (m, 1H) 6.42 (s, 2H) 6.85 (d, J = 7.80 Hz, 1H) |
| | 6.93 (td, J = 7.51, 1.75 Hz, 1H) 7.02-7.15 (m, 3H) 7.33 (d, J = 7.80 Hz, 1H) |
| | Mol. Wt. 356.4632; MS [M + 1] m/z 357.1 |
| 11 | 1H NMR (400 MHz, DMSO-d6) δ ppm 1.14-1.40 (m, 4H) 1.48-1.68 (m, 1H) |
| | 1.70-1.89 (m, 2H) 2.01-2.23 (m, 4H) 2.56-2.78 (m, 4H) 3.15 (d, J = 12.09 Hz, |
| | 1H) 3.14 (none, 2H) 3.68 (s, 3H) 3.84-3.95 (m, 1H) 3.94-4.04 (m, 1H) |
| | 6.42 (s, 2H) 6.73-6.88 (m, 2H) 6.89-7.02 (m, 2H) 7.33 (d, J = 7.80 Hz, 1H) |
| | Mol. Wt. 356.4632; MS [M + 1] m/z 357.1 |
| 12 | 1H NMR (400 MHz, DMSO-d6) δ ppm 1.20-1.35 (m, 1H) 1.51-1.66 (m, 1H) |
| | 1.70-1.87 (m, 2H) 2.10-2.15 (m, 1H) 2.16 (s, 3H) 2.57-2.75 (m, 2H) |
| | 3.09-3.20 (m, J = 12.09 Hz, 1H) 3.25-3.31 (m, 1H) 3.72 (s, 3H) 3.86-3.95 (m, 1H) |
| | 3.95-4.03 (m, 1H) 6.43 (s, 2H) 6.87 (d, J = 8.19 Hz, 1H) 6.98-7.10 (m, 2H) |
| | 7.21 (d, J = 2.34 Hz, 1H) 7.36 (d, J = 7.80 Hz, 1H) |
| | Mol. Wt. 362.8547; MS [M + 1] m/z 363 |
| 13 | 1H NMR (400 MHz, DMSO-d6) δ ppm 1.13-1.27 (m, 1H) 1.47-1.64 (m, 1H) |
| | 1.67-1.80 (m, 2H) 2.08 (s, 1H) 2.24 (s, 3H) 2.52-2.71 (m, 2H) 3.07-3.16 (m, |
| | J = 11.70 Hz, 1H) 3.20 (dd, J = 11.89, 2.53 Hz, 1H) 3.85-3.93 (m, 1H) |
| | 3.92-4.00 (m, 1H) 6.42 (s, 2H) 7.00 (d, J = 7.80 Hz, 1H) 7.03-7.11 (m, 2H) 7.38-7.49 (m, |
| | 3H) |
| | Mol. Wt. 332.8289; MS [M + 1] m/z 333 |
| 14 | 1H NMR (400 MHz, DMSO-d6) δ ppm 1.16-1.36 (m, 1H) 1.48-1.65 (m, 1H) |
| | 1.68-1.85 (m, 2H) 2.11 (s, 1H) 2.18 (s, 3H) 2.33 (s, 3H) 2.56-2.73 (m, 2H) |
| | 3.10-3.17 (m, J = 11.70 Hz, 1H) 3.88-3.96 (m, 1H) 3.97-4.05 (m, 1H) 6.42 (s, 2H) |
| | 6.93 (d, J = 7.80 Hz, 1H) 7.07 (d, J = 8.19 Hz, 1H) 7.16 (dd, J = 8.38, 1.75 Hz, 1H) |
| | 7.35-7.39 (m, J = 1.95 Hz, 1H) 7.41 (d, J = 7.80 Hz, 1H) |
| | Mol. Wt. 346.8557; MS [M + 1] m/z 347 |
| 15 | 1H NMR (400 MHz, DMSO-d6) δ ppm 1.11-1.28 (m, 1H) 1.49-1.64 (m, 1H) |
| | 1.66-1.77 (m, 2H) 2.10 (s, 1H) 2.26 (s, 3H) 2.52-2.59 (m, 1H) 2.59-2.70 (m, |
| | 1H) 3.15 (dd, J = 20.08, 12.67 Hz, 2H) 3.85-3.93 (m, 1H) 3.93-4.00 (m, 1H) |
| | 6.42 (s, 2H) 6.99 (dd, J = 8.19, 2.34 Hz, 1H) 7.03 (d, J = 8.19 Hz, 1H) 7.12 (t, |
| | J = 2.14 Hz, 1H) 7.18-7.23 (m, 1H) 7.39 (t, J = 8.19 Hz, 1H) 7.46 (d, J = 8.19 Hz, 1H) |
| | Mol. Wt. 332.8289; MS [M + 1] m/z 333.2 |
| 16 | 1H NMR (400 MHz, DMSO-d6) δ ppm 1.14 (d, J = 7.03 Hz, 6H) 1.19-1.31 (m, 1H) |
| | 1.52-1.65 (m, 1H) 1.69-1.82 (m, 2H) 2.13 (broad (br.) s., 1H) 2.20 (s, 3H) |
| | 2.25 (s, 3H) 2.56-2.71 (m, 2H) 2.91-3.04 (m, 1H) 3.10-3.19 (m, J = 12.10 Hz, 1H) |
| | 3.24 (dd, J = 12.10, 3.12 Hz, 1H) 3.92 (dd, J = 9.90, 5.60 Hz, 1H) 3.99 (dd, |
| | J = 9.90, 7.10 Hz, 1H) 6.43 (s, 2H) 6.90-6.96 (m, 2H) 7.34 (s, 1H) 7.40 (d, |
| | J = 7.81 Hz, 1H) 7.40 (d, J = 7.81 Hz, 1H) |
| | Mol. Wt. 388.9361; MS [M + 1] m/z 389.2 |
| 17 | 1H NMR (400 MHz, DMSO-d6) δ ppm 1.12-1.30 (m, 1H) 1.50-1.66 (m, 1H) |
| | 1.67-1.80 (m, 2H) 2.10 (br. s., 1H) 2.26 (s, 3H) 2.63 (none, 7H) 2.53-2.73 (m, |
| | 2H) 3.10-3.18 (m, J = 12.49 Hz, 1H) 3.22 (dd, J = 12.49, 3.12 Hz, 1H) 3.91 (dd, |
| | J = 9.80, 7.00 Hz, 1H) 3.98 (dd, J = 9.80, 5.50 Hz, 1H) 6.43 (s, 2H) 7.01-7.05 (m, |
| | J = 8.20 Hz, 1H) 7.07 (dd, J = 8.79, 2.93 Hz, 1H) 7.40 (none, 3H) 7.47 (d, J = 8.20 Hz, |
| | 1H) 7.62 (d, J = 8.59 Hz, 1H) |
| | Mol. Wt. 367.274; MS [M + 1] m/z 367.1 |
| 18 | 1H NMR (400 MHz, DMSO-d6) δ ppm 1.14-1.28 (m, 1H) 1.50-1.65 (m, 1H) |
| | 1.67-1.78 (m, 2H) 2.03 (dt, J = 14.82, 7.41 Hz, 2H) 2.11 (br. s., 1H) 2.23 (s, 3H) |
| | 2.56 (t, J = 11.70 Hz, 1H) 2.64 (td, J = 12.28, 2.73 Hz, 1H) 3.11-3.17 (m, J = 12.09 Hz, |
| | 1H) 3.20 (dd, J = 12.48, 3.12 Hz, 1H) 3.88 (dd, J = 9.80, 7.20 Hz, 1H) 3.96 (dd, |
| | J = 9.80, 5.30 Hz, 1H) 6.43 (s, 2H) 6.76 (dd, J = 8.19, 2.34 Hz, 1H) 6.86 (d, J = 1.95 Hz, |
| | 1H) 7.17 (d, J = 8.19 Hz, 1H) 7.39 (d, J = 7.80 Hz, 1H) |
| | Mol. Wt. 338.4484; MS [M + 1] m/z 339.2 |
| 19 | 1H NMR (400 MHz, DMSO-d6) δ ppm 1.14-1.30 (m, 1H) 1.53-1.65 (m, 1H) |
| | 1.66-1.79 (m, 6H) 2.13 (br. s., 1H) 2.20 (s, 3H) 2.54-2.59 (m, 1H) 2.64 (td, |
| | J = 12.38, 2.92 Hz, 1H) 2.75 (br. s., 2H) 3.10-3.18 (m, J = 12.09 Hz, 1H) 3.21 (dd, |
| | J = 12.28, 2.92 Hz, 1H) 3.87 (dd, J = 9.80, 6.80 Hz, 1H) 3.96 (dd, J = 9.80, 5.50 Hz, |
| | 1H) 6.42 (s, 2H) 6.67 (d, J = 7.80 Hz, 1H) 6.87 (d, J = 7.41 Hz, 1H) 6.91 (d, J = 8.19 Hz, |
| | 1H) 7.37 (d, J = 8.19 Hz, 1H) |
| | Mol. Wt. 352.4752; MS [M + 1] m/z 353.2 |
| 20 | 1H NMR (400 MHz, DMSO-d6) δ ppm 1.14-1.27 (m, 1H) 1.21 (d, J = 6.63 Hz, 6H) |
| | 1.49-1.63 (m, 1H) 1.67-1.79 (m, 2H) 2.03-2.15 (m, 1H) 2.23 (s, 3H) |
| | 2.58 (t, J = 11.80 Hz, 1H) 2.61-2.70 (m, 1H) 2.83-2.95 (m, J = 6.80 Hz, 1H) 3.13 (d, |
| | J = 12.09 Hz, 1H) 3.22 (dd, J = 12.48, 3.12 Hz, 1H) 3.39 (br. s., 2H) 3.88 (dd, |
| | J = 9.80, 7.20 Hz, 1H) 3.95 (dd, J = 9.90, 6.60 Hz, 1H) 6.42 (s, 2H) 6.90-6.99 (m, |
| | 3H) 7.19-7.26 (m, 2H) 7.41 (d, J = 8.19 Hz, 1H) |
| | Mol. Wt. 340.4642; MS [M + 1] m/z 341.2 |
| 21 | 1H NMR (400 MHz, DMSO-d6) δ ppm 1.24-1.39 (m, 1H) 1.52-1.68 (m, 1H) |
| | 1.73-1.81 (m, 1H) 1.81-1.90 (m, 1H) 2.13 (s, 3H) 2.17 (br. s., 1H) |
| | 2.60-2.78 (m, 2H) 3.16 (d, J = 12.09 Hz, 1H) 3.31 (d, J = 12.09 Hz, 1H) 3.74 (s, 3H) |
| | 3.88-3.97 (m, 1H) 3.97-4.06 (m, 1H) 6.42 (s, 2H) 6.87 (d, J = 7.80 Hz, 1H) |
| | 6.89-6.96 (m, J = 9.16, 9.16 Hz, 1H) 6.98 (d, J = 8.58 Hz, 1H) 7.17-7.27 (m, 1H) 7.37 (d, |
| | J = 7.80 Hz, 1H) |
| | Mol. Wt. 346.3997; MS [M + 1] m/z 347.1 |
| 22 | 1H NMR (400 MHz, DMSO-d6) δ ppm 1.14-1.35 (m, 1H) 1.46-1.66 (m, 1H) |
| | 1.66-1.86 (m, 2H) 2.12 (br. s., 1H) 2.19 (s, 3H) 2.55-2.74 (m, 2H) 3.14 (d, |
| | J = 12.09 Hz, 1H) 3.25 (dd, J = 12.28, 2.92 Hz, 1H) 3.87-3.96 (m, 1H) |
| | 3.97-4.05 (m, 1H) 6.42 (s, 2H) 6.97 (d, J = 8.58 Hz, 1H) 7.23 (d, J = 8.97 Hz, 1H) |
| | 7.40-7.49 (m, 2H) 7.73 (d, J = 2.73 Hz, 1H) |
| | Mol. Wt. 367.274; MS [M + 1] m/z 367 |
| 23 | 1H NMR (400 MHz, DMSO-d6) δ ppm 1.16-1.39 (m, 1H) 1.50-1.69 (m, 1H) |
| | 1.69-1.87 (m, 2H) 2.15 (br. s., 1H) 2.18 (s, 3H) 2.58-2.75 (m, 2H) 3.15 (d, |
| | J = 12.48 Hz, 1H) 3.28 (dd, J = 12.09, 3.12 Hz, 1H) 3.89-3.97 (m, 1H) |
| | 3.97-4.05 (m, 1H) 6.42 (s, 2H) 6.95 (d, J = 8.19 Hz, 1H) 7.27-7.34 (m, 2H) 7.43 (d, J = 8.19 Hz, |
| | 1H) 7.56-7.62 (m, 1H) |
| | Mol. Wt. 350.819; MS [M + 1] m/z 351 |
| 24 | 1H NMR (400 MHz, DMSO-d6) δ ppm 1.15-1.37 (m, 1H) 1.57 (q, J = 12.48 Hz, 1H) |
| | 1.67-1.88 (m, 2H) 2.12 (br. s., 1H) 2.17 (s, 3H) 2.56-2.73 (m, 2H) 3.13 (d, |
| | J = 11.70 Hz, 1H) 3.23-3.30 (m, 1H) 3.88-3.96 (m, 1H) 3.97-4.04 (m, 1H) |
| | 6.41 (s, 2H) 6.94 (d, J = 8.19 Hz, 1H) 7.21-7.30 (m, 2H) 7.43 (d, J = 7.80 Hz, 1H) |
| | 7.57 (dd, J = 8.77, 2.14 Hz, 1H) |
| | Mol. Wt. 350.819; MS [M + 1] m/z 351 |
| 25 | 1H NMR (400 MHz, DMSO-d6) δ ppm 1.16-1.40 (m, 1H) 1.48-1.66 (m, 1H) |
| | 1.68-1.88 (m, 2H) 2.13 (br. s., 1H) 2.17 (s, 3H) 2.57-2.78 (m, 2H) 3.14 (d, |
| | J = 12.09 Hz, 1H) 3.23-3.31 (m, 1H) 3.87-3.96 (m, 1H) 3.96-4.04 (m, 1H) |
| | 6.42 (s, 2H) 6.93 (d, J = 7.80 Hz, 1H) 7.07-7.15 (m, 1H) 7.28-7.36 (m, 1H) |
| | 7.38-7.45 (m, 2H) |
| | Mol. Wt. 334.364; MS [M + 1] m/z 335 |
| 26 | 1H NMR (400 MHz, DMSO-d6) δ ppm 1.20-1.38 (m, 1H) 1.52-1.68 (m, 1H) |
| | 1.70-1.88 (m, 2H) 2.16 (s, 3H) 2.59-2.78 (m, 2H) 3.16 (d, J = 12.48 Hz, 1H) |
| | 3.30 (dd, J = 12.09, 3.12 Hz, 1H) 3.79 (s, 3H) 3.89-3.96 (m, 1H) 3.98-4.05 (m, 1H) |
| | 6.42 (s, 2H) 6.89 (d, J = 7.80 Hz, 1H) 6.93 (dd, J = 8.97, 3.12 Hz, 1H) 7.12 (d, |
| | J = 3.12 Hz, 1H) 7.15 (d, J = 8.97 Hz, 1H) 7.39 (d, J = 8.19 Hz, 1H) |
| | Mol. Wt. 362.8547; MS [M + 1] m/z 363 |
| 27 | 1H NMR (400 MHz, DMSO-d6) δ ppm 1.10-1.26 (m, 1H) 1.47-1.64 (m, 1H) |
| | 1.65-1.77 (m, 2H) 2.08 (br. s., 1H) 2.25 (s, 3H) 2.51-2.58 (m, 1H) |
| | 2.59-2.68 (m, 1H) 3.13 (d, J = 12.48 Hz, 1H) 3.19 (dd, J = 12.09, 3.12 Hz, 1H) 3.87-3.94 (m, |
| | 1H) 3.94-4.00 (m, 1H) 6.42 (s, 2H) 7.02-7.06 (m, J = 7.41 Hz, 1H) 7.34 (dd, |
| | J = 7.99, 2.14 Hz, 1H) 7.39 (br. s., 1H) 7.48 (d, J = 7.80 Hz, 1H) 7.49-7.53 (m, 1H) |
| | Mol. Wt. 366.3809; MS [M + 1] m/z 367.2 |
| 28 | 1H NMR (400 MHz, DMSO-d6) δ ppm 1.21-1.37 (m, 1H) 1.54-1.67 (m, 1H) |
| | 1.71-1.88 (m, 2H) 2.15 (s, 3H) 2.16 (br. s., 1H) 2.60-2.76 (m, 2H) 3.17 (d, |
| | J = 12.09 Hz, 1H) 3.30 (dd, J = 11.50, 2.53 Hz, 1H) 3.70 (s, 3H) 3.86-3.95 (m, 1H) |
| | 3.96-4.04 (m, 1H) 6.43 (s, 2H) 6.72-6.81 (m, 1H) 6.85 (d, J = 7.80 Hz, 1H) |
| | 7.01-7.13 (m, 2H) 7.34 (d, J = 7.80 Hz, 1H) |
| | Mol. Wt. 346.3997; MS [M + 1] m/z 347 |
| 29 | 1H NMR (400 MHz, DMSO-d6) δ ppm 1.22-1.38 (m, 1H) 1.54-1.70 (m, 1H) |
| | 1.72-1.89 (m, 1H) 2.17 (br. s., 4H) 2.60-2.79 (m, 2H) 3.17 (d, J = 12.48 Hz, 2H) |
| | 3.30 (dd, J = 12.09, 3.12 Hz, 2H) 3.90-3.98 (m, 1H) 3.98-4.06 (m, 1H) 6.43 (s, 2H) |
| | 6.96 (d, J = 7.80 Hz, 1H) 7.41-7.47 (m, 1H) 7.58-7.66 (m, 1H) 7.69-7.79 (m, |
| | 1H) |
| | Mol. Wt. 352.3541; MS [M + 1] m/z 353 |
| 30 | 1H NMR (400 MHz, DMSO-d6) δ ppm 1.18-1.40 (m, 1H) 1.52-1.70 (m, 1H) |
| | 2.16 (s, 3H) 2.18 (br. s., 1H) 2.61-2.77 (m, 2H) 3.16 (d, J = 12.09 Hz, 1H) |
| | 3.30 (dd, J = 12.09, 2.73 Hz, 2H) 3.78 (s, 3H) 3.89-3.95 (m, 1H) 3.97-4.03 (m, 1H) |
| | 6.42 (s, 2H) 6.74-6.80 (m, 1H) 6.89 (d, J = 8.58 Hz, 1H) 6.96 (dd, J = 12.48, 3.12 Hz, |
| | 1H) 7.17 (t, J = 8.97 Hz, 1H) 7.38 (d, J = 7.80 Hz, 1H) |
| | Mol. Wt. 346.3997; MS [M + 1] m/z 347 |
| 31 | 1H NMR (400 MHz, DMSO-d6) δ ppm 1.11-1.30 (m, 1H) 1.48-1.64 (m, 1H) |
| | 1.66-1.80 (m, 2H) 2.09 (br. s., 1H) 2.27 (s, 3H) 2.57 (t, J = 11.70 Hz, 1H) |
| | 2.65 (td, J = 12.28, 2.73 Hz, 1H) 3.13 (d, J = 12.48 Hz, 1H) 3.20 (dd, J = 12.48, 3.51 Hz, 1H) |
| | 3.87-3.94 (m, 1H) 3.94-4.01 (m, 1H) 6.43 (s, 2H) 6.90-6.96 (m, 1H) |
| | 7.05 (d, J = 7.80 Hz, 1H) 7.23 (dd, J = 10.53, 2.73 Hz, 1H) 7.48 (d, J = 8.19 Hz, 1H) |
| | 7.57 (t, J = 8.77 Hz, 1H) |
| | Mol. Wt. 350.819; MS [M + 1] m/z 351 |
| 32 | 1H NMR (400 MHz, DMSO-d6) δ ppm 1.00-1.16 (m, 1H) 1.44-1.58 (m, 1H) |
| | 1.58-1.71 (m, 1H) 1.95 (br. s., 1H) 2.44 (t, J = 11.70 Hz, 1H) 2.53-2.62 (m, 1H) |
| | 3.06-3.15 (m, J = 12.48 Hz, 1H) 3.64-3.69 (m, 3H) 3.71-3.79 (m, 1H) |
| | 3.79-3.88 (m, 4H) 6.41 (s, 2H) 6.47 (d, J = 3.12 Hz, 1H) 6.67 (dd, J = 8.97, 3.12 Hz, 1H) |
| | 6.71 (d, J = 8.58 Hz, 1H) 6.91 (dd, J = 8.58, 2.73 Hz, 1H) 7.04 (d, J = 8.97 Hz, 1H) |
| | 7.18 (d, J = 2.73 Hz, 1H) |
| | Mol. Wt. 377.8656; MS [M + 1] m/z 378.2 |
| 33 | 1H NMR (400 MHz, DMSO-d6) δ ppm 0.95-1.09 (m, 1H) 1.41-1.53 (m, 1H) |
| | 1.53-1.69 (m, 2H) 1.92 (br. s., 1H) 2.40 (t, J = 11.70 Hz, 1H) 2.50-2.60 (m, 1H) |
| | 3.02 (dd, J = 11.50, 3.70 Hz, 1H) 3.08 (d, J = 12.87 Hz, 1H) 3.81-3.92 (m, 2H) |
| | 6.42 (s, 2H) 6.91 (t, J = 8.97 Hz, 1H) 7.17-7.22 (m, 2H) 7.23-7.30 (m, 2H) |
| | 7.60 (dd, J = 10.92, 2.34 Hz, 1H) |
| | Mol. Wt. 370.2492; MS [M + 1] m/z 369.9 |
| 34 | 1H NMR (400 MHz, DMSO-d6) δ ppm 0.99-1.17 (m, 1H) 1.44-1.59 (m, 1H) |
| | 1.60-1.73 (m, 2H) 1.99 (br. s., 1H) 2.45 (t, J = 11.89 Hz, 1H) 2.52-2.62 (m, 1H) |
| | 3.10 (d, J = 12.09 Hz, 2H) 3.81 (s, 3H) 3.82-3.88 (m, 1H) 3.88-3.94 (m, 1H) |
| | 6.41 (s, 2H) 6.83 (d, J = 8.58 Hz, 1H) 6.87 (d, J = 2.34 Hz, 1H) 6.92-6.97 (m, 1H) |
| | 7.12-7.17 (m, 2H) 7.22 (d, J = 2.34 Hz, 1H) |
| | Mol. Wt. 382.2849; MS [M + 1] m/z 382.1 |
| 35 | 1H NMR (400 MHz, DMSO-d6) δ ppm 1.01-1.21 (m, 1H) 1.48-1.60 (m, 1H) |
| | 1.61-1.73 (m, 2H) 2.44-2.51 (m, 1H) 2.54-2.64 (m, 1H) 3.09-3.18 (m, |
| | J = 12.48 Hz, 2H) 3.81 (s, 3H) 3.92-3.98 (m, 1H) 3.98-4.05 (m, 1H) 6.43 (s, 2H) |
| | 6.84 (d, J = 8.58 Hz, 1H) 6.93-6.98 (m, 1H) 7.13 (d, J = 2.34 Hz, 1H) 7.24 (d, |
| | J = 2.34 Hz, 1H) 7.30 (d, J = 8.19 Hz, 1H) 7.49 (dd, J = 8.58, 1.56 Hz, 1H) |
| | Mol. Wt. 415.8369; MS [M + 1] m/z 416 |
| 36 | 1H NMR (400 MHz, DMSO-d6) δ ppm 1.20-1.39 (m, 1H) 1.52-1.69 (m, 1H) |
| | 1.70-1.87 (m, 2H) 2.13 (br. s., 1H) 2.59-2.76 (m, 2H) 3.16 (d, J = 12.09 Hz, 1H) |
| | 3.28 (dd, J = 11.89, 2.92 Hz, 1H) 3.77 (s, 3H) 3.84-3.90 (m, 1H) 3.91-3.97 (m, 1H) |
| | 6.43 (s, 2H) 6.60-6.65 (m, 1H) 6.86 (dd, J = 12.48, 3.12 Hz, 1H) |
| | 6.99-7.01 (m, 2H) 7.11 (t, J = 9.36 Hz, 1H) 7.24 (t, J = 1.17 Hz, 1H) |
| | Mol. Wt. 365.8299; MS [M + 1] m/z 366 |
| 37 | 1H NMR (400 MHz, DMSO-d6) δ ppm 1.06-1.27 (m, 1H) 1.44-1.63 (m, 1H) |
| | 1.63-1.84 (m, 2H) 1.94-2.17 (m, 1H) 2.56-2.71 (m, 1H) 3.01-3.20 (m, 2H) |
| | 3.78 (s, 3H) 3.89-4.13 (m, 2H) 6.42 (s, 2H) 6.73 (d, J = 2.92 Hz, 1H) |
| | 6.90-6.99 (m, 2H) 7.11 (d, J = 10.72 Hz, 1H) |
| | Mol. Wt. 399.3819; MS [M + 1] m/z 400 |
| 38 | 1H NMR (400 MHz, DMSO-d6) δ ppm 0.87-1.15 (m, 1H) 1.37-1.71 (m, 3H) |
| | 1.84-2.07 (m, 1H) 2.36 (t, J = 11.60 Hz, 1H) 2.56-2.58 (m, 1H) 3.05 (dd, J = 43.47, |
| | 12.67 Hz, 2H) 3.77-4.01 (m, 2H) 6.43 (s, 2H) 6.69 (dd, J = 9.07, 4.97 Hz, 1H) |
| | 7.01 (s, 1H) 7.04 (s, 1H) 7.05-7.13 (m, 1H) 7.21-7.34 (m, 1H) 7.56 (dd, |
| | J = 8.38, 3.12 Hz, 1H) |
| | Mol. Wt. 353.7942; MS [M + 1] m/z 354 |
| 39 | 1H NMR (400 MHz, DMSO-d6) δ ppm 1.19-1.43 (m, 1H) 1.50-1.68 (m, 1H) |
| | 1.68-1.95 (m, 2H) 2.04-2.29 (m, 1H) 2.58-2.86 (m, 2H) 3.03-3.36 (m, 2H) |
| | 3.74-4.13 (m, 2H) 6.43 (s, 2H) 6.60-6.78 (m, 1H) 7.00 (dd, J = 12.18, 2.83 Hz, 1H) |
| | 7.08-7.21 (m, 2H) 7.24 (dd, J = 8.09, 3.02 Hz, 1H) 7.62 (dd, J = 8.38, 3.12 Hz, 1H) |
| | Mol. Wt. 353.7942; MS [M + 1] m/z 354 |
| 40 | 1H NMR (400 MHz, DMSO-d6) δ ppm 1.15-1.34 (m, 2H) 1.47-1.65 (m, 1H) |
| | 1.67-1.85 (m, 2H) 2.02-2.18 (m, 1H) 3.07-3.17 (m, 1H) 3.19-3.27 (m, |
| | J = 14.04 Hz, 1H) 3.86-3.95 (m, 1H) 3.95-4.04 (m, 1H) 6.42 (s, 1H) 6.49 (s, 1H) |
| | 6.99 (t, J = 9.45 Hz, 1H) 7.04 (d, J = 2.53 Hz, 1H) 7.06 (d, J = 2.34 Hz, 1H) |
| | 7.08-7.15 (m, 1H) 7.19-7.38 (m, 1H) |
| | Mol. Wt. 365.8299; MS [M + 1] m/z 366 |
| 41 | White solid, Elemental Analysis: Calculated for C₁₈H₂₁F₁N₂O₂ × C₄H₄O₄ (432.453): |
| | C, 61.10; H, 5.83; N, 6.48. Found: C, 61.01; H, 5.77; N, 6.26, MS (APCI+) m/z |
| | 317.1 [M + 1, 33%]. |
| 42 | 1H NMR (400 MHz, DMSO-d6) δ ppm 1.17-1.47 (m, 1H) 1.52-1.76 (m, 1H) |
| | 1.75-1.98 (m, 2H) 2.09-2.19 (m, 3H) 2.20-2.42 (m, 1H) 2.65-2.90 (m, 2H) |
| | 3.19-3.29 (m, 1H) 3.34-3.45 (m, 1H) 3.88-4.02 (m, 2H) 6.96 (d, J = 8.59 Hz, 1H) |
| | 7.15-7.29 (m, 2H) 7.30-7.39 (m, 1H) 7.46 (d, J = 8.00 Hz, 1H) 8.64 (s, 1H) |
| | Mol. Wt. 334.14; MS [M + 1] m/z 335 |
| 43 | White solid, mp 134-136° C. Elemental Analysis: Calculated for C₁₈H₂₁NO₂ × HCl |
| | (319.834): C, 67.60; H, 6.93; N, 4.38. Found: C, 67.27; H, 7.01; N, 4.35. MS |
| | (APCI⁺) m/z 284.1 [parent + 1, 100%]. |
| 44 | 1H NMR (400 MHz, DMSO-d6) δ ppm 1.22-1.46 (m, 1H) 1.57-1.75 (m, 1H) |
| | 1.76-1.94 (m, 2H) 2.17 (s, 3H) 2.21-2.37 (m, J = 17.96, 13.47 Hz, 1H) |
| | 2.66-2.88 (m, 2H) 3.17-3.28 (m, J = 15.91, 3.03 Hz, 2H) 3.94 (m, 1H) 4.04 (m, 1H) |
| | 6.97 (d, J = 8.20 Hz, 1H) 7.29-7.43 (m, 2H) 7.46 (d, J = 8.39 Hz, 1H) 8.71 (s, 1H) |
| | Mol. Wt. 352.14; MS [M + 1] m/z 353 |
| 45 | 1H NMR (400 MHz, DMSO-d6) δ ppm 1.22-1.44 (m, 1H) 1.57-1.75 (m, 1H) |
| | 1.77-1.92 (m, 2H) 2.17 (s, 3H) 2.20-2.31 (m, 1H) 2.69-2.89 (m, 2H) |
| | 3.18-3.28 (m, J = 12.69 Hz, 1H) 3.33-3.46 (m, 1H) 3.92-4.02 (m, J = 17.18 Hz, 1H) |
| | 4.03-4.13 (m, 1H) 6.99 (d, J = 8.00 Hz, 1H) 7.47 (d, J = 8.00 Hz, 1H) |
| | 7.51-7.66 (m, J = 7.42 Hz, 2H) 8.70 (br. s., 2H) |
| | Mol. Wt. 368; MS [M + 3] m/z 371 |
| 46 | Mp 128-130° C. Elemental Analysis: Calculated for C₁₈H₂₀FNO₂•C₄H₄O₄ |
| | (417.438): C, 63.30; H, 5.80; N, 3.36. Found: C, 62.90; H, 5.66; N, 3.30. MS |
| | (APCI⁺) m/z 302.2 [parent + 1, 100%]. |
| 47 | 1H NMR (400 MHz, DMSO-d6) δ ppm 1.30-1.34 (m, 1H) 1.65 (m, 1H) |
| | 1.73-1.88 (m, 2H) 2.11 (s, 3H) 2.17-2.34 (m, 1H) 2.65-2.85 (m, 2H) 3.17-3.27 (m, 1H) |
| | 3.34-3.37 (m, 1H) 3.94-3.98 (m, 1H) 4.04-4.15 (m, 1H) 6.91 (d, J = 8.39 Hz, 1H) |
| | 7.21-7.36 (m, 1H) 7.43 (d, J = 8.00 Hz, 1H) 7.56 (d, J = 8.00 Hz, 2H) 8.65 (br. |
| | s., 1H) |
| | Mol. Wt. 366; MS [M + 1] m/z 367 |
| 48 | 1H NMR (400 MHz, DMSO-d6) δ ppm 1.15-1.44 (m, 1H) 1.54-1.74 (m, 1H) |
| | 1.75-1.90 (m, 2H) 2.07-2.16 (m, 3H) 2.19-2.32 (m, 1H) 2.67-2.84 (m, 2H) |
| | 3.17-3.28 (m, 1H) 3.32-3.43 (m, 1H) 3.93-3.98 (m, 1H) 4.04-4.14 (m, 1H) |
| | 6.93 (d, J = 8.59 Hz, 1H) 7.44 (d, J = 8.00 Hz, 1H) 7.65 (d, J = 8.39 Hz, 2H) 8.62 (br. s., 2H) |
| | Mol. Wt. 384; MS [M + 1] m/z 385 |
| 49 | 1H NMR (400 MHz, DMSO-d6) δ ppm 1.21-1.47 (m, 1H) 1.59-1.77 (m, 1H) |
| | 1.77-1.90 (m, 2H) 2.18 (s, 3H) 2.24-2.40 (m, 1H) 2.67-2.84 (m, 2H) 3.24 (m, |
| | 1H) 3.36 (m, 1H) 3.92-4.03 (m, 1H) 4.04-4.14 (m, 1H) 7.00 (d, J = 8.00 Hz, 1H) |
| | 7.27-7.37 (m, 1H) 7.46-7.55 (m, 2H) 8.92 (br. s., 2H) |
| | Mol. Wt. 352; MS [M + 1] m/z 353 |
| 50 | 1H NMR (400 MHz, DMSO-d6) δ ppm 1.14-1.46 (m, 1H) 1.72 (m, 1H) |
| | 1.77-2.00 (m, 2H) 2.17 (s, 3H) 2.24-2.42 (m, 1H) 2.62-2.87 (m, 2H) 3.23 (m, 1H) |
| | 3.36 (m, 1H) 3.91-4.03 (m, 1H) 4.03-4.12 (m, 1H) 7.00 (d, J = 8.00 Hz, 1H) |
| | 7.29-7.43 (m, 1H) 7.49 (d, J = 8.00 Hz, 1H) 7.52-7.64 (m, 1H) 8.90 (br. s., 1H) |
| | Mol. Wt. 368; MS [M + 1] m/z 369 |
| 51 | White solid, Elemental Analysis: Calculated for C₁₈H₂₂N₂O₃ × C₄H₄O₄ (430.462): C, |
| | 61.39; H, 6.09; N, 6.51. Found: C, 61.04; H, 6.16; N, 6.39, MS (APCI+) m/z |
| | 315.2 [M + 1, 100%]. |
| 52 | White solid, Elemental Analysis: Calculated for C₁₈H₂₂N₂O₂ × C₄H₄O₄ (414.462): C, |
| | 63.76; H, 6.32; N, 6.76. Found: C, 63.56; H, 6.36; N, 6.63, MS (APCI+) m/z |
| | 299.2 [M + 1, 100%]. |
| 53 | White solid, Elemental Analysis: Calculated for C₁₈H₁₉F₁N₂O₂ × C₄H₄O₄ (418.426): |
| | C, 60.28; H, 5.54; N, 6.69. Found: C, 59.96; H, 5.55; N, 6.55, MS (APCI+) m/z |
| | 303.2 [M + 1, 100%]. |
| 54 | White solid, Elemental Analysis: Calculated for C₁₈H₁₈F₂N₂O₂ × C₄H₄O₄ (436.416): |
| | C, 57.80; H, 5.08; N, 6.42. Found: C, 57.48; H, 5.06; N, 6.24, MS (APCI+) m/z |
| | 321.2 [M + 1, 100%]. |
| 55 | White solid, Elemental Analysis: Calculated for C₁₇H₁₉Cl₁N₂O₂ × C₄H₄O₄ (434.880): |
| | C, 58.00; H, 5.33; N, 6.44. Found: C, 57.98; H, 5.31; N, 6.28, MS (APCI+) m/z |
| | 319.1 [M + 1, 100%], 321.2 [M + 3, 33%]. |
| 56 | White solid, mp 172-173° C. Elemental Analysis: Calculated for C₁₇H₁₉FN₂O₂ × C₄H₄O₄ |
| | (418.426): C, 60.28; H, 5.54; N, 6.69. Found: C, 60.34; H, 5.58; N, 6.66. |
| 57 | White solid, Elemental Analysis: Calculated for C₁₈H₁₉N₃O₂ × C₄H₄O₄ × (463.595): |
| | C, 62.11; H, 5.45; N, 9.88. Found: C, 61.95; H, 5.35; N, 9.57, MS (APCI+) m/z |
| | 310.1 [M + 1, 62%]. |
| 58 | Off white solid, Elemental Analysis: Calculated for C₁₉H₁₈N₄O₂ × C₄H₄O₄ × 0.73 |
| | H₂O (463.595): C, 59.59; H, 5.10; N, 12.09. Found: C, 59.78; H, 4.74; N, 11.69, |
| | MS (APCI+) m/z 335.2 [M + 1, 11%]. |
| 59 | Off-white solid, mp 128-129° C. Elemental Analysis: Calculated for C₁₇H₁₉ClN₂O₂ × C₄H₄O₄ |
| | (434.880): C, 58.00; H, 5.33; N, 6.44. Found: C, 57.93; H, 5.08; N, 6.44. |
| 60 | White solid, mp 148-150° C. Elemental Analysis: Calculated for C₁₇H₁₈F₂N₂O₂ × C₄H₄O₄ |
| | (436.416): C, 57.80; H, 5.08; N, 6.42. Found: C, 57.91; H, 5.00; N, 6.32. |
| 61 | White solid, Elemental Analysis: Calculated for C₁₈H₁₈F₁N₃O₂ × C₄H₄O₄ × 0.26 H₂O |
| | (448.109): C, 58.97; H, 5.07; N, 9.38. Found: C, 58.59; H, 5.00; N, 9.48, MS |
| | (APCI+) m/z 328.1 [M + 1, 100%]. |
| 62 | White solid, Elemental Analysis: Calculated for C₁₉H₂₁N₃O₃ × C₄H₄O₄ (455.472): C, |
| | 60.65; H, 5.53; N, 9.23. Found: C, 60.72; H, 5.62; N, 9.01, MS (APCI+) m/z |
| | 340.1 [M + 1, 100%]. |
| 63 | White solid, mp 130-131° C. Elemental Analysis: Calculated for C₁₇H₁₈F₂N₂O₂ × C₄H₄O₄ |
| | (436.416): C, 57.80; H, 5.08; N, 6.42. Found: C, 57.67; H, 4.94; N, 6.26. |
| 64 | White solid, mp 160-162° C. Elemental Analysis: Calculated for C₁₇H₁₈C₁₂N₂O₂ × C₄H₄O₄ |
| | (469.325): C, 53.74; H, 4.72; N, 5.97. Found: C, 53.90; H, 4.43; N, 5.94. |
| 65 | White solid, mp 169-170° C. Elemental Analysis: Calculated for C₁₉H₂₄N₂O₂ × C₄H₄O₄ |
| | (428.489): C, 64.47; H, 6.59; N, 6.54. Found: C, 64.26; H, 6.52; N, 6.59. |
| 66 | White solid, mp 143-144° C., dec. Elemental Analysis: Calculated for |
| | C₁₇H₁₈ClFN₂O₂ × C₄H₄O₄ (452.871): C, 55.70; H, 4.90; N, 6.19. Found: C, 55.73; |
| | H, 4.89; N, 5.95. |
| 67 | White solid, mp 136-138° C. Elemental Analysis: Calculated for C₁₈H₂₁FN₂O₂ × C₄H₄O₄ |
| | (432.453): C, 61.10; H, 5.83; N, 6.48. Found: C, 60.81; H, 5.82; N, 6.38. |
| 68 | White solid, mp 130-132° C. Elemental Analysis: Calculated for C₁₈H₂₂N₂O₂ × C₄H₄O₄ |
| | (414.462): C, 63.76; H, 6.32; N, 6.76. Found: C, 63.37; H, 6.25; N, 6.65. |
| 69 | White solid, mp 150-151° C. Elemental Analysis: Calculated for C₁₇H₁₈ClFN₂O₂ × C₄H₄O₄ |
| | (452.871): C, 55.70; H, 4.90; N, 6.19. Found: C, 55.57; H, 4.86; N, 6.25. |
| 70 | White solid, mp 144-146° C. Elemental Analysis: Calculated for C₁₈H₁₉F₃N₂O₃ × C₄H₄O₄ |
| | (484.433): C, 54.55; H, 4.79; N, 5.78. Found: C, 54.25; H, 4.63; N, 5.60. |
| 71 | White solid, mp 189-190° C., dec. Elemental Analysis: Calculated for |
| | C₁₇H₁₈ClFN₂O₂ × C₄H₄O₄ (452.871): C, 55.70; H, 4.90; N, 6.19. Found: C, 55.61; |
| | H, 4.57; N, 6.08. |
| 72 | White solid, mp 182-183° C., dec. Elemental Analysis: Calculated for C₁₇H₁₈F₂N₂O₂ × C₄H₄O₄ |
| | (436.416): C, 57.80; H, 5.08; N, 6.42. Found: C, 57.62; H, 5.14; N, 6.33. |
| 73 | White solid, mp 165-167° C., dec. Elemental Analysis: Calcd for C₁₈H₂₂N₂O₂ × C₄H₄O₄ |
| | (414.462): C, 63.76; H, 6.32; N, 6.76. Found: C, 63.47; H, 6.46; N, 6.72. |
| 74 | White solid, mp 129-134° C., dec. Elemental Analysis: Calcd for C₂₀H₂₆N₂O₃ × C₄H₄O₄ |
| | (458.516): C, 62.87; H, 6.59; N, 6.11. Found: C, 62.81; H, 6.53; N, 6.03. |
| 75 | White solid, mp 112-114° C., dec. Elemental Analysis: Calcd for C₂₀H₂₆N₂O₂ × C₄H₄O₄ |
| | (442.517): C, 65.14; H, 6.83; N, 6.33. Found: C, 64.95; H, 6.93; N, 6.42. |
| 76 | White solid, mp 183-184° C., dec. Elemental Analysis: Calcd for C₁₈H₂₁ClN₂O₂ × C₄H₄O₄ |
| | (448.907): C, 58.86; H, 5.61; N, 6.24. Found: C, 58.74; H, 5.52; N, 6.11. |
| 77 | Mp 165-167° C., dec. Elemental Analysis: Calculated for C₁₈H₂₂N₂O₂ × C₄H₄O₄ |
| | (414.462): C, 63.76; H, 6.32; N, 6.76. Found: C, 63.50; H, 6.29; N, 6.70. |
| 78 | White solid, Elemental Analysis: Calculated for C₁₅H₂₄N₂O₂ × C₄H₄O₄ (380.445): C, |
| | 59.99; H, 7.42; N, 7.36. Found: C, 59.96; H, 7.50; N, 7.08, MS (APCI+) m/z |
| | 265.1 [M + 1, 43%]. |
| 79 | White solid, Elemental Analysis: Calculated for C₁₃H₂₀N₂O₂ × C₄H₄O₄ (352.391): C, |
| | 57.94; H, 6.86; N, 7.95. Found: C, 58.15; H, 7.02; N, 7.83, MS (APCI+) m/z |
| | 237.2 [M + 1, 3%]. |
| 80 | White solid, Elemental Analysis: Calculated for C₁₄H₂₂N₂O₂ × C₄H₄O₄ (366.418): C, |
| | 59.00; H, 7.15; N, 7.65. Found: C, 58.83; H, 7.43; N, 7.32, MS (APCI+) m/z |
| | 251.2 [M + 1, 5%]. |
| 81 | White solid, Elemental Analysis: Calculated for C₁₇H₂₆N₂O₂ × C₄H₄O₄ (406.483): C, |
| | 62.05; H, 7.44; N, 6.89. Found: C, 61.86; H, 7.63; N, 6.81, MS (APCI+) m/z |
| | 291.2 [M + 1, 1%]. |
| 82 | White solid, mp 146-148° C., dec. Elemental Analysis: Calculated for C₁₉H₂₄N₂O₂ × C₄H₄O₄ |
| | (428.489): C, 64.47; H, 6.59; N, 6.54. Found: C, 64.43; H, 6.53; N, 6.44. |
| 83 | White solid, mp 141-142° C., dec. Elemental Analysis: Calculated for C₂₀H₂₆N₂O₂ × C₄H₄O₄ |
| | (442.517): C, 65.14; H, 6.83; N, 6.33. Found: C, 65.14; H, 7.01; N, 6.26. |
| 84 | Elemental Analysis: Calculated for C₁₉H₂₄N₂O₂ × C₄H₄O₄ × 0.20 H₂O (432.081): C, |
| | 63.93; H, 6.63; N, 6.48. Found: C, 63.56; H, 6.55; N, 6.35, MS (APCI+) m/z |
| | 313.2 [M + 1, 77%]. |
| 85 | White solid, Elemental Analysis: Calculated for C₁₉H₂₃F₁N₂O₂ × C₄H₄O₄ × 0.10 H₂O |
| | (448.270): C, 61.62; H, 6.12; N, 6.25. Found: C, 61.24; H, 6.01; N, 6.17, MS |
| | (APCI+) m/z 331.2 [M + 1, 100%]. |
| 86 | White solid, Elemental Analysis: Calculated for C₁₅H₂₄N₂O₂ × C₄H₄O₄ × 0.48 H₂O |
| | (417.136): C, 59.99; H, 7.42; N, 7.36. Found: C, 59.98; H, 7.42; N, 7.33, MS |
| | (APCI+) m/z 265.1 [M + 1, 83%]. |
| 87 | White solid. Elemental Analysis: Calculated for C₁₇H₂₈N₂O₂ × C₄H₄O₄ × 0.48 H₂O |
| | (417.136): C, 60.47; H, 7.96; N, 6.72. Found: C, 60.07; H, 7.78; N, 6.37, MS |
| | (APCI+) m/z 293.1 [M + 1, 10%]. |
| 88 | Mp 191-192° C. Elemental Analysis: Calculated for C₂₃H₂₃FN₂O₂ × C₄H₄O₄ × 0.19 |
| | H₂O (497.947): C, 65.13; H, 5.54; N, 5.62. Found: C, 65.13; H, 5.59; N, 5.35. |
| 89 | White solid, mp 150-152° C. Elemental Analysis: Calculated for C₂₃H₂₃FN₂O₂ × C₄H₄O₄ |
| | (494.524): C, 65.58; H, 5.50; N, 5.66. Found: C, 65.32; H, 5.50; N, 5.68. |
| 90 | White solid, mp 159-161° C. Elemental Analysis: Calculated for C₁₉H₂₄N₂O₂ × C₄H₄O₄ |
| | (428.489): C, 64.47; H, 6.59; N, 6.54. Found: C, 64.44; H, 6.60; N, 6.47. |
| 91 | Hygroscopic white solid, mp 58-68° C., dec. MS (APCI+) m/z 313.5 [parent + 1, |
| | 100%]. Elemental Analysis: Calculated for C₁₉H₂₄N₂O₂ × C₄H₄O₄ (428.489): C, |
| | 64.47; H, 6.59; N, 6.54. Found: C, 63.36; H, 6.69; N, 7.21. |
| 92 | White solid, mp 186-188° C. Elemental Analysis: Calculated for C₂₁H₂₈N₂O₂ × C₄H₄O₄ |
| | (456.544): C, 65.77; H, 7.07; N, 6.14. Found: C, 65.75; H, 7.07; N, 6.03. |
| 93 | Elemental Analysis: Calculated for C₁₇H₂₀N₂O₂ × C₄H₄O₄ (400.435): C, 62.99; H, |
| | 6.04; N, 7.00. Found: C, 62.69; H, 5.99; N, 6.97, MS (APCI+) m/z 285.1 [M + 1, |
| | 38%]. |
| 94 | White solid, mp 141-143° C., dec. Elemental Analysis: Calculated for C₁₇H₂₀N₂O₂ × C₄H₄O₄ |
| | (400.435): C, 62.99; H, 6.04; N, 7.00. Found: C, 62.87; H, 5.96; N, 6.88. |
| 95 | Elemental Analysis: Calculated for C₁₈H₂₂N₂O₂ × C₄H₄O₄ (414.462): C, 63.76; H, |
| | 6.32; N, 6.76. Found: C, 63.50; H, 6.29; N, 6.65, MS (APCI+) m/z 299.1 [M + 1, |
| | 95%]. |
| 96 | Elemental Analysis: Calculated for C₁₈H₁₉F₂NO₂ × C₄H₄O₄ (435.429): C, 60.69; H, |
| | 5.32; N, 3.22. Found: C, 60.53; H, 5.52; N, 2.93, MS (APCI+) m/z 320.0 [M + 1, |
| | 48%], 1H NMR (400 MHz, DMSO-d₆) δ ppm 1.11-1.30 (m, 1H) 1.49-1.65 (m, 1H) |
| | 1.70 (d, J = 11.52 Hz, 2H) 2.04 (s, 1H) 2.53-2.71 (m, 2H) 3.14 (d, J = 13.08 Hz, |
| | 1H) 3.21 (dd, J = 12.40, 3.42 Hz, 1H) 3.84-3.99 (m, 2H) 6.43 (s, 2H) |
| | 6.79-6.84 (m, 1H) 7.00-7.06 (m, 2H) 7.08-7.14 (m, 2H) 7.17-7.24 (m, 2H). |
| 97 | Elemental Analysis: Calculated for C₁₈H₁₈F₃NO₂ × C₄H₄O₄ (453.419): C, 58.28; H, |
| | 4.89; N, 3.09. Found: C, 58.12; H, 4.92; N, 3.09, MS (APCI+) m/z 338.0 [M + 1, |
| | 25%], 1H NMR (400 MHz, DMSO-d₆) δ ppm 1.10-1.29 (m, 1H) 1.46-1.62 (m, 1H) |
| | 1.69 (d, J = 11.33 Hz, 2H) 2.00 (s, 1H) 2.53-2.71 (m, 2H) 3.08-3.25 (m, 2H) |
| | 3.82-4.01 (m, 2H) 6.43 (s, 2H) 6.76-6.85 (m, J = 8.98, 4.98, 1.71, 1.71 Hz, 1H) |
| | 6.88-6.94 (m, 1H) 7.09-7.22 (m, 3H) 7.42 (dt, J = 10.40, 9.25 Hz, 1H). |
| 98 | Elemental Analysis: Calculated for C₁₈H₁₉F₂NO₂ × C₂H₁F₃O₂ (433.369): C, 55.43; |
| | H, 4.65; N, 3.23. Found: C, 55.27; H, 4.38; N, 3.17, MS (APCI+) m/z 320.0 [M + 1, |
| | 16%], 1H NMR (400 MHz, DMSO-d₆) δ ppm 0.97-1.19 (m, 1H) 1.43-1.64 (m, 3H) |
| | 1.67-1.78 (m, 1H) 2.01 (s, 1H) 2.42-2.48 (m, 1H) 2.62 (td, J = 12.55, 3.42 Hz, |
| | 1H) 3.03 (dd, J = 12.30, 3.51 Hz, 1H) 3.20 (d, J = 12.11 Hz, 1H) 3.86 (dd, |
| | J = 9.67, 6.74 Hz, 1H) 3.93-4.02 (m, 1H) 6.84-6.93 (m, 2H) 6.97-7.06 (m, 2H) |
| | 7.10-7.19 (m, 2H) 7.26 (td, J = 8.49, 6.44 Hz, 1H) 8.46 (S, 2H). |
| 99 | Mp 137-140° C., dec. Elemental Analysis: Calculated for C₁₉H₂₃NO₂ × HCl × 0.36 |
| | Et₂O (360.546): C, 68.09; H, 7.72; N, 3.88. Found: C, 67.70; H, 7.75; N, 4.03. |
| | MS (APCI⁺) m/z 298.1 [parent + 1, 100%]. |
| 100 | Off-white solid, mp 145-147° C. Elemental Analysis: Calculated for C₁₄H₂₁NO₂ × C₄H₄O₄ |
| | (351.403): C, 61.52; H, 7.17; N, 3.99. Found: C, 61.41; H, 7.28; N, 3.95. |
| | MS (APCI⁺) m/z 236.2 [parent + 1, 100%]. |
| 101 | Mp 149-150° C. Elemental Analysis: Calculated for C₁₈H₂₇NO₂ × HCl (325.882): |
| | C, 66.34; H, 8.66; N, 4.30. Found: C, 66.06; H, 8.92; N, 4.24. MS (APCI+) m/z |
| | 290.2 [parent + 1, 100%]. |
| 102 | Mp 162° C. (shrink). Elemental Analysis: Calculated for C₁₆H₂₅NO₂ × HCl |
| | (299.844): C, 64.09; H, 8.74; N, 4.67. Found: C, 64.04; H, 8.96; N, 4.61, MS |
| | (APCI+) m/z 264.1 [M + 1, 100%], 1H NMR (400 MHz, DMSO-d₆) δ ppm 0.99 (d, |
| | J = 6.59 Hz, 6H) 1.27-1.42 (m, 1H) 1.59-1.75 (m, 1H) 1.82 (d, J = 10.75 Hz, 2H) |
| | 1.95-2.08 (m, 1H) 2.21 (s, 1H) 2.75 (t, J = 11.84 Hz, 2H) 3.24 (d, J = 12.46 Hz, 1H) |
| | 3.72 (d, J = 6.59 Hz, 2H) 3.82 (dd, J = 9.65, 7.20 Hz, 1H) 3.94 (dd, J = 9.65, 5.25 Hz, |
| | 1H) 6.83-6.92 (m, 2H) 6.93-7.02 (m, 2H) 8.81 (s, 2H) |
| 103 | Elemental Analysis: Calculated for C₁₃H₁₈F₁NO₂ × C₄H₄O₄ (355.366): C, 57.46; H, |
| | 6.24; N, 3.94. Found: C, 57.51; H, 6.30; N, 3.84, MS (APCI+) m/z 240.1 [M + 1, |
| | 62%], 1H NMR (400 MHz, DMSO-d₆) δ ppm 1.22-1.37 (m, 1H) 1.52-1.69 (m, 1H) |
| | 1.71-1.85 (m, 2H) 2.08 (s, 1H) 2.62-2.75 (m, 2H) 3.17 (d, J = 12.50 Hz, 1H) |
| | 3.35 (dd, J = 12.50, 3.51 Hz, 1H) 3.76-3.84 (m, 4H) 3.86-3.94 (m, 1H) 6.42 (s, 2H) |
| | 6.83 (ddd, J = 10.30, 8.54, 1.46 Hz, 1H) 6.88 (dt, J = 8.45, 1.44 Hz, 1H) 7.05 (td, |
| | J = 8.40, 6.25 Hz, 1H). |
| 104 | Elemental Analysis: Calculated for C₁₆H₂₄FNO₂ × C₄H₄O₄ (397.448): C, 60.44; H, |
| | 7.10; N, 3.52. Found: C, 60.35; H, 7.16; N, 3.44, MS (APCI+) m/z 282.0 [M + 1, |
| | 65%], 1H NMR (400 MHz, DMSO-d₆) δ ppm 0.99 (d, J = 6.83 Hz, 6H) |
| | 1.25-1.40 (m, 1H) 1.55-1.71 (m, 1H) 1.72-1.86 (m, 2H) 1.97-2.19 (m, 2H) |
| | 2.64-2.78 (m, 2H) 3.18 (d, J = 12.69 Hz, 1H) 3.35 (dd, J = 12.11, 3.32 Hz, 1H) 3.78 (d, J = 6.44 Hz, |
| | 2H) 3.81 (dd, J = 9.57, 6.83 Hz, 1H) 3.89-3.95 (m, 1H) 6.43 (s, 2H) |
| | 6.78-6.89 (m, 2H) 7.02 (td, J = 8.45, 6.35 Hz, 1H). |
| 105 | Elemental Analysis: Calculated for C₁₄H₂₀FNO₂ × C₄H₄O₄ (369.385): C, 58.53; H, |
| | 6.55; N, 3.79. Found: C, 58.51; H, 6.76; N, 3.72, MS (APCI+) m/z 254.1 [M + 1, |
| | 100%], 1H NMR (400 MHz, DMSO-d₆) δ ppm 1.27-1.33 (m, 1H) 1.35 (t, J = 6.93 Hz, |
| | 3H) 1.56-1.69 (m, 1H) 1.70-1.86 (m, 2H) 2.09 (s, 1H) 2.64-2.76 (m, 2H) |
| | 3.17 (d, J = 12.30 Hz, 1H) 3.37 (dd, J = 12.30, 3.51 Hz, 1H) 3.80 (dd, J = 9.76, 7.03 Hz, |
| | 1H) 3.91 (dd, J = 9.76, 5.27 Hz, 1H) 4.06 (q, J = 7.03 Hz, 2H) 6.42 (s, 2H) |
| | 6.79-6.88 (m, 2H) 7.03 (td, J = 8.40, 6.25 Hz, 1H). |
| 106 | Mp 82-88° C., dec. Elemental Analysis: Calculated for C₂₀H₂₅NO₂ × C₄H₄O₄ × 0.40 |
| | H₂O (434.708): C, 66.31: H, 6.91; N, 3.22. Found: C, 66.32; H, 6.67; N, 3.45. |
| | MS (APCI⁺) m/z 312.2 [parent + 1, 100%]. |
| 107 | Elemental Analysis: Calculated for C₁₅H₂₃NO₂ (249.356): C, 72.25; H, 9.30; N, |
| | 5.62. Found: C, 72.00; H, 9.19; N, 5.41, MS (APCI+) m/z 250.1 [M + 1, 17%], 1H |
| | NMR (400 MHz, CDCl₃) δ ppm 1.26 (d, J = 6.34 Hz, 3H) 1.29-1.39 (m, 1H) |
| | 1.40-1.46 (m, 4H) 1.46-1.62 (m, 2H) 1.67-1.86 (m, 2H) 2.04 (s, 1H) 2.57 (s, 2H) |
| | 2.97-3.07 (m, 1H) 3.15 (s, 1H) 4.06 (dd, J = 6.91 Hz, 1H) 4.10-4.17 (m, 1H) |
| | 6.85-6.95 (m, 4H) |
| 108 | Elemental Analysis: Calculated for C₂₀H₂₅NO₂ × C₂H₂O × 0.14 H₂O (403.975): C, |
| | 65.41; H, 6.81; N, 3.47. Found: C, 65.02; H, 6.93; N, 3.41, MS (APCI+) m/z |
| | 312.2 [M + 1, 40%], 1H NMR (400 MHz, CDCl₃) δ ppm 1.26 (d, J = 6.34 Hz, 3H) |
| | 1.29-1.52 (m, 5H) 1.67-1.86 (m, 2H) 2.00-2.08 (m, 1H) 2.51-2.63 (m, 2H) |
| | 2.98-3.07 (m, 1H) 3.11-3.20 (m, 1H) 4.03-4.10 (m, 2H) 4.13 (ddd, J = 12.32, 6.34, |
| | 6.22 Hz, 1H) 6.83-6.96 (m, 4H). |
| 109 | Elemental Analysis: Calculated for C₁₇H₂₇NO₂ (277.410): C, 73.61; H, 9.81; N, |
| | 5.05. Found: C, 73.41; H, 9.94; N, 4.83, MS (APCI+) m/z 278.2 [M + 1, 100%], 1H |
| | NMR (400 MHz, CDCl₃) δ ppm 1.05 (dd, J = 6.59, 2.44 Hz, 6H) 1.25 (d, J = 6.35 Hz, |
| | 3H) 1.32-1.53 (m, 2H) 1.69-1.85 (m, 2H) 1.97-2.08 (m, 1H) 2.14 (ddd, |
| | J = 20.09, 13.37, 6.84 Hz, 1H) 2.50-2.62 (m, 2H) 3.03 (dd, J = 12.21, 0.98 Hz, 1H) |
| | 3.15 (d, J = 11.72 Hz, 1H) 3.74 (ddd, J = 13.07, 8.91, 6.59 Hz, 2H) 4.16 (ddd, |
| | J = 12.15, 6.35, 6.17 Hz, 1H) 6.82-6.95 (m, 4H). |
| 110 | Elemental Analysis: Calculated for C₁₈H₂₇NO₂ × C₂H₂O (379.457): C, 63.61; H, |
| | 7.70; N, 3.69. Found: C, 63.14; H, 7.80; N, 3.62, MS (APCI+) m/z 290.2 [M + 1, |
| | 100%], 1H NMR (400 MHz, DMSO-d₆) δ ppm 1.15 (d, J = 6.35 Hz, 3H) |
| | 1.31-1.46 (m, 1H) 1.53-1.69 (m, 1H) 1.78-2.13 (m, 8H) 2.68-2.87 (m, 3H) 3.24 (d, |
| | J = 11.97 Hz, 1H) 3.30 (dd, J = 12.21, 2.20 Hz, 1H) 3.91 (d, J = 6.35 Hz, 2H) |
| | 4.25 (dt, J = 10.75, 6.23 Hz, 1H) 6.85 (td, J = 7.57, 1.71 Hz, 1H) 6.93 (td, J = 7.63, 1.59 Hz, |
| | 1H) 6.95-7.00 (m, 2H). |
| 111 | Elemental Analysis: Calculated for C₁₉H₂₉NO₂ × C₂H₂O × 0.05 H₂O (394.375): C, |
| | 63.96; H, 7.95; N, 3.55. Found: C, 63.57; H, 8.34; N, 3.40, MS (APCI+) m/z |
| | 304.2 [M + 1, 90%], 1H NMR (400 MHz, DMSO-d₆) δ ppm 1.16 (d, J = 6.35 Hz, 3H) |
| | 1.23-1.54 (m, 6H) 1.61 (d, J = 13.68 Hz, 1H) 1.66-1.77 (m, 2H) 1.78-1.93 (m, 4H) |
| | 1.95-2.07 (m, 1H) 2.68-2.87 (m, 2H) 3.24 (d, J = 12.46 Hz, 1H) 3.31 (dd, |
| | J = 12.09, 2.56 Hz, 1H) 4.23 (ddd, J = 12.39, 8.73, 3.79 Hz, 1H) 4.29 (ddd, J = 10.68, |
| | 6.47, 6.29 Hz, 1H) 6.83-6.93 (m, 2H) 6.99 (ddd, J = 7.51, 4.09, 2.08 Hz, 2H). |
| 112 | Elemental Analysis: Calculated for C₁₈H₂₉NO₂ × C₂H₂O 0.12 H₂O (383.625): C, |
| | 62.62; H, 8.21; N, 3.65. Found: C, 62.22; H, 8.38; N, 3.64, MS (APCI+) m/z |
| | 292.2 [M + 1, 40%], 1H NMR (400 MHz, DMSO-d₆) δ ppm 0.93 (d, J = 6.59 Hz, 6H) |
| | 1.15 (d, J = 6.35 Hz, 3H) 1.31-1.44 (m, 1H) 1.61 (q, J = 6.76 Hz, 3H) 1.74-1.92 (m, 3H) |
| | 1.95-2.06 (m, 1H) 2.69-2.84 (m, 2H) 3.24 (d, J = 12.70 Hz, 1H) 3.30 (dd, |
| | J = 12.58, 2.81 Hz, 1H) 3.96 (t, J = 6.59 Hz, 2H) 4.26 (dt, J = 10.81, 6.32 Hz, 2H) |
| | 6.85 (td, J = 7.57, 1.71 Hz, 1H) 6.92 (td, J = 7.69, 1.71 Hz, 1H) 6.98 (td, J = 8.24, |
| | 1.59 Hz, 2H). |
| 113 | Elemental Analysis: Calculated for C₁₆H₂₅N₁O₃ × HCl (315.844): C, 60.85; H, |
| | 8.30; N, 4.43. Found: C, 60.53; H, 8.48; N, 4.29, MS (APCI+) m/z 280.1 [M + 1, |
| | 100%], 1H NMR (400 MHz, DMSO-d₆) δ ppm 1.16 (d, J = 6.35 Hz, 3H) |
| | 1.33-1.47 (m, 1H) 1.64 (q, J = 13.59 Hz, 1H) 1.77-1.91 (m, 2H) 1.97-2.10 (m, 1H) 2.79 (s, |
| | 2H) 3.22 (d, J = 12.70 Hz, 1H) 3.30 (d, J = 14.41 Hz, 1H) 3.33-3.34 (m, 3H) |
| | 3.57-3.77 (m, 2H) 4.07 (t, J = 4.64 Hz, 2H) 4.27 (dt, J = 10.75, 6.35 Hz, 1H) 6.87 (td, |
| | J = 7.51, 1.59 Hz, 1H) 6.94 (td, J = 7.63, 1.83 Hz, 1H) 6.99 (ddd, J = 7.82, 3.91, 1.71 Hz, |
| | 2H) 8.97 (d, J = 98.42 Hz, 2H). |
| 114 | Elemental Analysis: Calculated for C₁₉H₂₄N₂O₂ × C₄H₄O₄ × 0.27 H₂O (433.342): |
| | C, 63.65; H, 6.64; N, 6.46. Found: C, 63.36; H, 6.82; N, 6.28, MS (APCI+) m/z |
| | 313.0 [M + 1, 48%], 1H NMR (400 MHz, DMSO-d₆) δ ppm 1.35 (t, J = 6.93 Hz, 3H) |
| | 1.43-1.61 (m, 2H) 1.78 (d, J = 13.28 Hz, 2H) 2.37 (s, 1H) 2.62-2.80 (m, 2H) |
| | 3.06-3.21 (m, 2H) 4.05 (q, J = 6.90 Hz, 2H) 5.16 (d, J = 5.47 Hz, 2H) 6.42 (s, 2H) |
| | 6.67-6.78 (m, 2H) 6.81-6.90 (m, 1H) 6.96 (dd, J = 8.10, 1.27 Hz, 1H) 7.31 (ddd, |
| | J = 7.52, 4.88, 1.07 Hz, 1H) 7.44 (d, J = 7.81 Hz, 1H) 7.80 (td, J = 7.71, 1.76 Hz, 1H) |
| | 8.56 (ddd, J = 4.83, 1.71, 0.88 Hz, 1H). |
| 115 | Elemental Analysis: Calculated for C₁₈H₂₁FN₂O₂ × C₄H₄O₄ × 0.04 H₂O (433.369): |
| | C, 61.00; H, 5.84; N, 6.47. Found: C, 60.61; H, 6.21; N, 6.23, MS (APCI+) m/z |
| | 317.1 [M + 1, 61%], 1H NMR (400 MHz, DMSO-d₆) δ ppm 1.38-1.67 (m, 2H) |
| | 1.69-2.07 (m, 2H) 2.41 (s, 1H) 2.69 (t, J = 11.91 Hz, 2H) 3.06 (dd, J = 72.06, 12.11 Hz, |
| | 2H) 3.76 (s, 3 H) 5.14 (d, J = 6.25 Hz, 1H) 6.42 (s, 2H) 6.72 (ddd, J = 10.35, 8.59, |
| | 1.37 Hz, 1H) 6.82 (d, J = 8.40 Hz, 1H) 6.97 (td, J = 8.49, 6.25 Hz, 1H) 7.30 (ddd, |
| | J = 7.52, 4.88, 1.07 Hz, 1H) 7.52 (d, J = 7.81 Hz, 1H) 7.81 (td, J = 7.66, 1.66 Hz, 1H) |
| | 8.33-8.63 (m, 1H). |
| 116 | Elemental Analysis: Calculated for C₁₈H₁₉F₃N₂O₂ × C₄H₄O₄ (468.434): C, 56.41; |
| | H, 4.95; N, 5.98. Found: C, 56.24; H, 4.84; N, 5.83, MS (APCI+) m/z 353.0 [M + 1, |
| | 37%], 1H NMR (400 MHz, DMSO-d₆) δ ppm 1.36-1.61 (m, 2H) 1.77 (d, J = 11.33 Hz, |
| | 2H) 2.40 (s, 1H) 2.64 (q, J = 12.37 Hz, 2H) 3.05-3.20 (m, 2H) 5.38 (d, |
| | J = 5.47 Hz, 1H) 6.43 (s, 2H) 6.87 (dd, J = 8.49, 1.27 Hz, 1H) 6.97 (dt, J = 7.81, 1.37 Hz, |
| | 1H) 7.19 (dt, J = 7.91, 1.56 Hz, 1H) 7.27 (d, J = 8.01 Hz, 1H) 7.30-7.39 (m, 2H) |
| | 7.81 (dt, J = 7.76, 1.86 Hz, 1H) 8.61 (d, J = 3.91 Hz, 1H). |
| 117 | Elemental Analysis: Calculated for C₁₈H₂₁FN₂O₂ × C₄H₄O₄ (432.170): C, 61.10; H, |
| | 5.83; N, 6.48. Found: C, 61.00; H, 5.84; N, 6.41, MS (APCI+) m/z 317.1 [M + 1, |
| | 100%], ¹H NMR (400 MHz, methanol-d₄) δ ppm 1.57-1.77 (m, 2H) 1.99 (t, |
| | J = 11.01 Hz, 2H) 2.40-2.54 (m, 1H) 2.94 (s, 1H) 2.99-3.07 (m, 2H) 3.86 (s, 2H) |
| | 5.23 (d, J = 5.46 Hz, 1H) 6.49 (dd, J = 9.94, 2.92 Hz, 1H) 6.60-6.66 (m, 1H) |
| | 6.68 (s, 1H) 6.95 (dd, J = 8.97, 5.26 Hz, 1H) 7.33-7.43 (m, 1H) 7.50 (d, J = 7.99 Hz, |
| | 1H) 7.79-7.94 (m, 1H) 8.54-8.65 (m, 1H). |
| 118 | Elemental Analysis: Calculated for C₁₇H₂₇NO₃ × C₄H₄O₄ × 0.07 H₂O (410.734): C, |
| | 61.41; H, 7.64; N, 3.41. Found: C, 61.03; H, 7.70; N, 3.34, MS (APCI+) m/z |
| | 294.2 [M + 1, 58%], 1H NMR (400 MHz, DMSO-d₆) δ ppm 1.14 (d, J = 16.36 Hz, 6H) |
| | 1.34-1.47 (m, 1H) 1.55-1.71 (m, 1H) 1.78-1.88 (m, 1H) 1.89-2.03 (m, 2H) |
| | 2.66-2.83 (m, 2H) 3.21 (d, J = 13.68 Hz, 1H) 3.57 (d, J = 13.19 Hz, 1H) 3.65-3.70 (m, 2H) |
| | 4.02 (dd, J = 5.50, 3.79 Hz, 2H) 6.42 (s, 2H) 6.84 (td, J = 7.51, 1.83 Hz, 1H) |
| | 6.95-7.08 (m, 3H). |

Accordingly, another embodiment is a compound selected from the group consisting of:
2-(4-fluoro-2-methyl-phenoxy)-6-methyl-3-(piperidin-3-ylmethoxy)-pyridine;
2-(2,6-dimethyl-phenoxy)-6-methyl-3-(piperidin-3-ylmethoxy)-pyridine;
2-(2-methoxy-phenoxy)-6-methyl-3-(piperidin-3-ylmethoxy)-pyridine;
2-(2-methoxy-4-methyl-phenoxy)-6-methyl-3-(piperidin-3-ylmethoxy)-pyridine;
2-(2-methyl-phenoxy)-6-methyl-3-(piperidin-3-ylmethoxy)-pyridine;
2-(4-methoxy-phenoxy)-6-methyl-3-(piperidin-3-ylmethoxy)-pyridine;
2-(2-chloro-phenoxy)-6-methyl-3-(piperidin-3-ylmethoxy)-pyridine;
2-(4-methyl-phenoxy)-6-methyl-3-(piperidin-3-ylmethoxy)-pyridine;
2-(2,3-dimethoxy-phenoxy)-6-methyl-3-(piperidin-3-ylmethoxy)-pyridine;
2-[2-(1-methyl-ethoxy)-phenoxy]-6-methyl-3-(piperidin-3-ylmethoxy)-pyridine;
2-(4-ethyl-2-methoxy-phenoxy)-6-methyl-3-(piperidin-3-ylmethoxy)-pyridine;
2-(4-chloro-2-methoxy-phenoxy)-6-methyl-3-(piperidin-3-ylmethoxy)-pyridine;
2-(4-chloro-phenoxy)-6-methyl-3-(piperidin-3-ylmethoxy)-pyridine;
2-(2-chloro-4-methyl-phenoxy)-6-methyl-3-(piperidin-3-ylmethoxy)-pyridine;
2-(3-chloro-phenoxy)-6-methyl-3-(piperidin-3-ylmethoxy)-pyridine;
2-[4-chloro-5-methyl-2-(1-methyl-ethyl)-phenoxy]-6-methyl-3-(piperidin-3-ylmethoxy)-pyridine;
2-(3,4-dichloro-phenoxy)-6-methyl-3-(piperidin-3-ylmethoxy)-pyridine;
2-(2,3-dihydro-1H-inden-5-yloxy)-6-methyl-3-(piperidin-3-ylmethoxy)pyridine;
6-methyl-3-(piperidin-3-ylmethoxy)-2-(5,6,7,8-tetrahydronaphthalen-1-yloxy)-pyridine;
2-[4-(1-methyl-ethyl)-phenoxy]-6-methyl-3-(piperidin-3-ylmethoxy)-pyridine;
2-(2-fluoro-6-methoxy-phenoxy)-6-methyl-3-(piperidin-3-ylmethoxy)-pyridine;
2-(2,4-dichloro-phenoxy)-6-methyl-3-(piperidin-3-ylmethoxy)-pyridine;
2-(4-chloro-2-fluoro-phenoxy)-6-methyl-3-(piperidin-3-ylmethoxy)-pyridine;
2-(2-chloro-4-fluoro-phenoxy)-6-methyl-3-(piperidin-3-ylmethoxy)-pyridine;
2-(2,4-difluoro-phenoxy)-6-methyl-3-(piperidin-3-ylmethoxy)-pyridine;
2-(2-chloro-4-methoxy-phenoxy)-6-methyl-3-(piperidin-3-ylmethoxy)-pyridine;
6-methyl-2-(3-trifluoromethyl-phenoxy)-3-(piperidin-3-ylmethoxy)-pyridine;
2-(4-fluoro-2-methoxy-phenoxy)-6-methyl-3-(piperidin-3-ylmethoxy)-pyridine;
6-methyl-2-(2,4,5-trifluoro-phenoxy)-3-(piperidin-3-ylmethoxy)-pyridine;
2-(2-fluoro-4-methoxy-phenoxy)-6-methyl-3-(piperidin-3-ylmethoxy)-pyridine;
2-(4-chloro-3-fluoro-phenoxy)-6-methyl-3-(piperidin-3-ylmethoxy)-pyridine;
3-[2-(4-chloro-2-methoxy-phenoxy)-4-methoxy-phenoxymethyl]-piperidine;
3-[4-chloro-2-(4-chloro-2-fluoro-phenoxy)-phenoxymethyl]-piperidine;
3-[4-chloro-2-(4-chloro-2-methoxy-phenoxy)-phenoxymethyl]-piperidine;
3-[2-(4-chloro-2-methoxy-phenoxy)-4-trifluoromethyl-phenoxymethyl]-piperidine;
3-[2-(4-chloro-2-methoxy-phenoxy)-4-fluoro-phenoxymethyl]-piperidine;
3-[2-(4-fluoro-2-methoxy-phenoxy)-4-trifluoromethyl-phenoxymethyl]-piperidine;
3-[2-(2-chloro-4-fluoro-phenoxy)-3-fluoro-phenoxymethyl]-piperidine;
3-[2-(2-chloro-4-fluoro-phenoxy)-4-fluoro-phenoxymethyl]-piperidine;
3-[4-chloro-2-(2-fluoro-6-methoxy-phenoxy)-phenoxymethyl]-piperidine;
2-(4-fluoro-phenoxy)-6-methyl-3-(piperidin-3-ylmethoxy)-pyridine;
2-(2,6-difluoro-phenoxy)-6-methyl-3-(piperidin-3-ylmethoxy)-pyridine;
3-(2-phenoxy-phenoxymethyl)-piperidine;
6-methyl-2-(2,4,6-trifluoro-phenoxy)-3-(piperidin-3-ylmethoxy)-pyridine;
2-(4-chloro-2,6-difluoro-phenoxy)-6-methyl-3-(piperidin-3-ylmethoxy)-pyridine;
3-(4-fluoro-2-phenoxy-phenoxymethyl)-piperidine;
2-(2,6-dichloro-phenoxy)-6-methyl-3-(piperidin-3-ylmethoxy)-pyridine;
2-(2,6-dichloro-4-fluoro-phenoxy)-6-methyl-3-(piperidin-3-ylmethoxy)-pyridine;
6-methyl-3-(piperidin-3-ylmethoxy)-2-(2,3,6-trifluoro-phenoxy)-pyridine;
2-(3-chloro-2,6-difluoro-phenoxy)-6-methyl-3-(piperidin-3-ylmethoxy)-pyridine;
2-(4-methoxy-phenoxy)-3-(piperidin-3-ylmethoxy)-pyridine;
2-(4-methyl-phenoxy)-3-(piperidin-3-ylmethoxy)-pyridine;
2-(4-fluoro-phenoxy)-3-(piperidin-3-ylmethoxy)-pyridine;
2-(2,4-difluoro-phenoxy)-3-(piperidin-3-ylmethoxy)-pyridine;
2-(4-chloro-phenoxy)-3-(piperidin-3-ylmethoxy)-pyridine;
2-(4-fluoro-phenoxy)-3-(piperidin-3-ylmethoxy)-pyridine;
4-[3-(piperidin-3-ylmethoxy)-pyridin-2-yloxy]-benzonitrile;
4-[3-(piperidin-3-ylmethoxy)-pyridin-2-yloxy]-phthalonitrile;
2-(3-chloro-phenoxy)-3-(piperidin-3-ylmethoxy)-pyridine;
2-(3,4-difluoro-phenoxy)-3-(piperidin-3-ylmethoxy)-pyridine;
2-fluoro-4-[3-(piperidin-3-ylmethoxy)-pyridin-2-yloxy]-benzonitrile;
3-methoxy-4-[3-(piperidin-3-ylmethoxy)-pyridin-2-yloxy]-benzonitrile;
2-(3,4-difluoro-phenoxy)-3-(piperidin-3-ylmethoxy)-pyridine;
2-(3,4-dichloro-phenoxy)-3-(piperidin-3-ylmethoxy)-pyridine;
2-(3,4-dimethyl-phenoxy)-3-(piperidin-3-ylmethoxy)-pyridine;
2-(3-chloro-4-fluoro-phenoxy)-3-(piperidin-3-ylmethoxy)-pyridine;
2-(4-fluoro-3-methyl-phenoxy)-3-(piperidin-3-ylmethoxy)-pyridine;
2-(3-methyl-phenoxy)-3-(piperidin-3-ylmethoxy)-pyridine;
2-(4-chloro-3-fluoro-phenoxy)-3-(piperidin-3-ylmethoxy)-pyridine;
3-(piperidin-3-ylmethoxy)-2-(3-trifluoromethoxy-phenoxy)-pyridine;
2-(2-chloro-4-fluoro-phenoxy)-3-(piperidin-3-ylmethoxy)-pyridine;
2-(2,6-difluoro-phenoxy)-3-(piperidin-3-ylmethoxy)-pyridine;
2-(2-methyl-phenoxy)-3-(piperidin-3-ylmethoxy)-pyridine;
2-(2-isopropoxy-phenoxy)-3-(piperidin-3-ylmethoxy)-pyridine;
2-(2-isopropyl-phenoxy)-3-(piperidin-3-ylmethoxy)-pyridine;
2-(2-chloro-5-methyl-phenoxy)-3-(piperidin-3-ylmethoxy)-pyridine;
2-benzyloxy-3-(piperidin-3-ylmethoxy)-pyridine;
2-isobutoxy-3-(piperidin-3-ylmethoxy)-pyridine;
2-ethoxy-3-(piperidin-3-ylmethoxy)-pyridine;
2-isopropoxy-3-(piperidin-3-ylmethoxy)-pyridine;
2-cyclohexyloxy-3-(piperidin-3-ylmethoxy)-pyridine;
2-phenethyloxy-3-(piperidin-3-ylmethoxy)-pyridine;
2-(3-phenyl-propoxy)-3-(piperidin-3-ylmethoxy)-pyridine;
2-phenoxy-3-(1-piperidin-3-yl-propoxy)-pyridine;
2-(4-fluoro-phenoxy)-3-(1-piperidin-3-yl-propoxy)-pyridine;
2-ethoxy-3-(1-piperidin-3-yl-propoxy)-pyridine;
2-isobutoxy-3-(1-piperidin-3-yl-propoxy)-pyridine;
2-(4-fluoro-phenoxy)-3-[(phenyl)-(piperidin-3-yl)-methoxy]-pyridine;
2-ethoxy-3-[(phenyl)-(piperidin-3-yl)-methoxy]-pyridine;
2-isobutoxy-3-[(phenyl)-(piperidin-3-yl)-methoxy]-pyridine;
2-phenoxy-3-(piperidin-3-ylmethoxy)-pyridine;
2-phenoxy-3-(piperidin-3-ylmethoxy)-pyridine;
6-methyl-2-phenoxy-3-(piperidin-3-ylmethoxy)-pyridine;
3-[2-fluoro-6-(4-fluoro-phenoxy)-phenoxymethyl]-piperidine;
3-[2-(3,4-difluoro-phenoxy)-6-fluoro-phenoxymethyl]-piperidine;
3-[3-fluoro-2-(4-fluoro-phenoxy)-phenoxymethyl]-piperidine;
3-(2-benzyloxy-phenoxymethyl)-piperidine;
3-(2-ethoxy-phenoxymethyl)-piperidine;
3-(2-cyclohexyloxy-phenoxymethyl)-piperidine;
3-(2-isobutoxy-phenoxymethyl)-piperidine
3-(2-fluoro-6-methoxy-phenoxymethyl)-piperidine;
3-(2-fluoro-6-isobutoxy-phenoxymethyl)-piperidine;
3-(2-ethoxy-6-fluoro-phenoxymethyl)-piperidine;
3-[(2-ethoxy-phenoxy)-phenyl-methyl]-piperidine;
3-[1-(2-ethoxy-phenoxy)-ethyl]-piperidine;
3-[1-(2-benzyloxy-phenoxy)-ethyl]-piperidine;
3-[1-(2-isobutoxy-phenoxy)-ethyl]-piperidine;
3-[1-(2-cyclobutylmethoxy-phenoxy)-ethyl]-piperidine;
3-[1-(2-cyclohexyloxy-phenoxy)-ethyl]-piperidine;
3-{1-[2-(3-methyl-butoxy)-phenoxy]-ethyl}-piperidine;
3-{1-[2-(2-methoxy-ethoxy)-phenoxy]-ethyl}-piperidine;
2-[{2-ethoxy-phenoxy}-piperidin-3-yl-methyl]-pyridine;
2-[{2-fluoro-6-methoxy-phenoxy}-piperidin-3-yl-methyl]-pyridine;
2-[piperidin-3-yl-{2-trifluoromethoxy-phenoxy}-methyl]-pyridine;
2-[{5-fluoro-2-methoxy-phenoxy}-piperidin-3-yl-methyl]-pyridine;
3-{1-[2-(2-methoxy-ethoxy)-phenoxy]-1-methyl-ethyl}-piperidine; or
   a pharmaceutically acceptable acid addition salt thereof.

### Biological Methods

Compounds and salts of the invention can be assayed for their ability to inhibit a norepinephrine transporter receptor, serotonin transporter receptor, or both the norepinephrine and serotonin transporter receptors by, for example, using conventional radioligand receptor transport assays. The receptors can be heterologously expressed in cell lines and the assays can be conducted with membrane preparations from the cell lines that express at least one of the transporter receptors. Examples of useful assays are provided in Biological Methods 1 and 2.

### Biological Method 1

### Human Norepinephrine (hNET) Receptor Binding

Cell pastes of human embryonic kidney 293 (HEK-293) cells transfected with a human norepinephrine transporter cDNA were prepared. The cell pastes were resuspended in 400 to 700 mL of Krebs-N-2-hydroxyethylpiperazine-N′-2-ethanesulfonic acid (HEPES) assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 seconds. Aliquots of membranes (5 mg/mL protein) were stored in liquid nitrogen until used.

The binding assay was set up in Beckman deep-well polypropylene plates with a total volume of 250 μL containing: test compound (concentration of 10⁻⁵M to 10⁻¹²M), cell membranes, and 50 pM [¹²⁵I]-RTI-55 ([¹²⁵I]-3 beta-(4-iodophenyl)tropan-2 beta-carboxylic acid methyl ester) (Perkin Elmer, NEX-272; specific activity 2200 Ci/mmol). The reaction was incubated by gentle agitation for 90 minutes at room temperature and was terminated by filtration through Whatman GF/C filter plates using a Brandel 96-well plate harvester. Scintillation fluid (100 μL) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to Kᵢ values using the Cheng-Prusoff equation. The Kᵢ values (nM) for the hNET are reported below in Table 13.

### Biological Method 2

### Human Serotonin (hSERT) Receptor Binding

Cell pastes of HEK-293 cells transfected with a human serotonin transporter cDNA were prepared. The cell pastes were resuspended in 400 to 700 ml of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at Setting 7 for 30 seconds. Aliquots of membranes (˜2.5 mg/mL protein) were stored in liquid nitrogen until used.

Assays were set up in FlashPlates pre-coated with 0.1% polyethyleneimine (PEI) in a total volume of 250 μL containing: test compound (concentration 10⁻⁵M to 10⁻¹²M), cell membranes, and 50 μM [¹²⁵I]-RTI-55 (Perkin Elmer, NEX-272; specific activity 2200 Ci/mmol). The reaction was incubated and gently agitated for 90 minutes at room temperature, and terminated by removal of assay volume. Plates were covered, and bound [¹²⁵I]-RTI-55 was determined using a Wallac Trilux Beta Plate Counter. Test compounds were run in duplicate, and specific binding was defined as the difference between binding in the presence and absence of 10 μM citalopram.

Excel and GraphPad Prism software were used for data calculation and analysis. IC₅₀ values were converted to Kᵢ values using the Cheng-Prusoff equation. The Kᵢ values (nM) for the hSERT are reported below in Table 13.

TABLE 13

| Ex. | hNET | hSERT | | hNET | hSERT |
|---|---|---|---|---|---|
| No. | Ki (nM) | Ki (nM) | Ex. No. | Ki (nM) | Ki (nM) |
| 1 | 28 | 580 | 24 | 2.7 | 160 |
| 2 | 17 | 10000 | 25 | 3.3 | 72 |
| 3 | 2.2 | 1200 | 26 | 18 | 860 |
| 4 | 7.4 | 170 | 27 | 310 | 560 |
| 5 | 8.4 | 2800 | 28 | 6.1 | 300 |
| 6 | 16 | 360 | 29 | 5.3 | 130 |
| 7 | 2.7 | 350 | 30 | 15 | 230 |
| 8 | 10.3 | 159 | 31 | 8.4 | 101 |
| 9 | 3200 | 10000 | 32 | 46 | 120 |
| 10 | 10.1 | 2600 | 33 | 32 | 36 |
| 11 | 18 | 180 | 34 | 16 | 21 |
| 12 | 8.7 | 120 | 35 | 250 | 35 |
| 13 | 8.9 | 140 | 36 | 830 | 110 |
| 14 | 2.8 | 40.0 | 37 | 77 | 22 |
| 15 | 9.8 | 287 | 38 | 3.6 | 1900 |
| 16 | 2600 | 1700 | 39 | 802 | 110 |
| 17 | 12 | 63 | 40 | 7.5 | 14 |
| 18 | 54 | 620 | 41 | 3.8 | 210 |
| 19 | 86 | 1900 | 42 | 3.6 | 110 |
| 20 | 4800 | 2400 | 43 | 0.7 | 2300 |
| 21 | 4.9 | 72 | 44 | 4.8 | 82 |
| 22 | 4.7 | 79 | 45 | 8.4 | 29 |
| 23 | 5.3 | 28 | 46 | 2 | 320 |
| 47 | 10.4 | >9200 | 48 | 61 | >9200 |
| 49 | 6.1 | 25 | 50 | 5.5 | 60.3 |
| 51 | 27 | 7300 | 52 | 4 | 5200 |
| 53 | 3 | 7050 | 54 | 3 | 2700 |
| 55 | 17 | >10000 | 56 | 330 | 3800 |
| 57 | 58 | >10000 | 58 | 1200 | 3990 |
| 59 | 4 | 6300 | 60 | 2 | 6600 |
| 61 | 140 | >10000 | 62 | 68 | >10000 |
| 63 | 140 | 803 | 64 | 17 | 860 |
| 65 | 7 | 2200 | 66 | 20 | 2900 |
| 67 | 6 | 3300 | 68 | 6 | 5800 |
| 69 | 4 | 5500 | 70 | 102 | 9700 |
| 71 | 2 | 6600 | 72 | 2 | 3300 |
| 73 | 4 | >10000 | 74 | 11 | >10000 |
| 75 | 760 | >10000 | 76 | 4 | 4800 |
| 77 | 6100 | 7200 | 78 | 54 | >10000 |
| 79 | 108 | >10000 | 80 | 102 | >10000 |
| 81 | 84 | 6134 | 82 | 210 | 3700 |
| 83 | 210 | 10 | 84 | 3 | 2030 |
| 85 | 5 | 2030 | 86 | 13 | 705 |
| 87 | 7 | 350 | 88 | 4 | 150 |
| 89 | 43 | 590 | 90 | 2 | 71 |
| 91 | 99 | 430 | 92 | 29 | 140 |
| 93 | 5 | 7500 | 94 | 410 | 4700 |
| 95 | 4 | 530 | 96 | 1 | 530 |
| 97 | 1 | 690 | 98 | 1 | 1300 |
| 99 | 11 | 4800 | 100 | 33 | 2600 |
| 101 | 20 | >9700 | 102 | 32 | 3960 |
| 103 | 45 | 1200 | 104 | 26 | 1900 |
| 105 | 32 | 2800 | 106 | 7 | 430 |
| 107 | 7 | 2700 | 108 | 3 | 730 |
| 109 | 3 | 1200 | 110 | 3 | 420 |
| 111 | 5 | 1800 | 112 | 16 | 790 |
| 113 | 17 | 3600 | 114 | 7 | 3700 |
| 115 | 4 | 5020 | 116 | 3 | 3700 |
| 117 | 16 | 4600 | 118 | 38 | 2500 |

Another embodiment is a compound of Formula (I), or a pharmaceutically acceptable acid addition salt thereof, having an hNET Ki (nM) of less than 10 nM. Another embodiment is a compound of Formula (I), or a pharmaceutically acceptable acid addition salt thereof, having an hSERT Ki (nM) of less than 50 nM.

Another embodiment is a compound of Formula (I), or a pharmaceutically acceptable acid addition salt thereof, having a ratio of hSERT Ki (nM) divided by hNET Ki (nM) of from >1 to 50. Another embodiment is a compound of Formula (I), or a pharmaceutically acceptable acid addition salt thereof, having a ratio of hSERT Ki (nM) divided by hNET Ki (nM) of >50. Another embodiment is a compound of Formula (I), or a pharmaceutically acceptable acid addition salt thereof, having a ratio of hSERT Ki (nM) divided by hNET Ki (nM) of from 0.1 to 5; in still another embodiment the ratio is from 0.1 to <1.

In all such embodiments, the hSERT Ki is determined according to Biological Method 2 and the hNET Ki is determined according to Biological Method 1. The ratios of hSERT Ki (nM) divided by hNET Ki (nM) for the compounds of Examples 1-118 may be determined from the data provided in Table 13.

Another embodiment of the present invention is a compound of Formula (I), or a pharmaceutically acceptable acid addition salt thereof, having a human dopamine reuptake (hDAT) binding Ki of >5,000 nM. The hDAT binding assay is run in a manner similar to the assays described in Biological Methods 1 and 2.

The compounds and salts thereof of the invention may be assayed for their ability to alleviate capsaicin-induced mechanical allodynia in a rat (e.g., Sluka, KA, (2002) J of Neuroscience, 22(13): 5687-5693). For example, a rat model of capsaicin-induced mechanical allodynia) was carried out as described in Biological Method 3.

### Biological Method 3

### Capsaicin-Induced Mechanical Allodynia Rat Model

On Day 0, male Sprague-Dawley rats (about 150 g each) in the dark cycle were placed in suspended wire-bottom cages and allowed to acclimate for 0.5 hour in a darkened, quiet room. The Day 0 paw withdrawal threshold (PWT) was determined on the left hind paw by Von Frey hair assessment using the Dixon up and down method. After assessment, the plantar muscle of the right hind paw was injected with 100 μL of capsaicin (0.25% weight/volume (w/v) in 10% ethanol, 10% Tween 80, in sterile saline).

On Day 6, the PWT of the left hind paw (contralateral from the injected paw) was determined for each animal. Animals on Day 6 with PWT≦11.7 g were considered allodynic responders and were regrouped so that the animals in each cage had similar mean PWT values.

On Day 7, the responders were dosed subcutaneously with 10 mg of the test compound per kg of rat body weight in vehicle, or were administered vehicle (10 mL/kg) alone. The vehicle was phosphate buffered saline containing 2% CREMOPHOR® EL (BASF). The contralateral (i.e., left hind paw) PWT values were determined at 1 hour after the single dose, with the investigator blinded to the dosing scheme. For each animal, the Day 6 PWT value was subtracted from the Day 7, 1 hour PWT value for the 10 mg/kg doses of test compound to give a delta PWT value (Delta PWT (drug)), which represents the change in PWT due to the 1 hour drug treatment. In the case of vehicle-alone treated animals, the Day 6 PWT value was subtracted from the Day 7, 1 hour PWT for the 10 mL/kg doses of vehicle and the values averaged (mean Delta PWT (vehicle)). In addition, the Day 6 PWT was subtracted from the Day 0 PWT to give the baseline level (Baseline) of allodynia present in each animal. Percent inhibition of allodynia of each animal, normalized for vehicle controls, was determined using the following formula:

$$Percent Inhibition of Allodynia = 100 \times \frac{( \begin{matrix}Delta PWT ( drug ) - \\ mean Delta PWT ( vehicle )\end{matrix} )}{( \begin{matrix}Baseline - \\ mean Delta PWT ( vehicle )\end{matrix} )} .$$

The mean percent inhibition of allodynia values for eight animals assayed per test compound are shown in Table 14. Compounds in Table 14 exhibiting a greater than 30% inhibition are considered to be active in the allodynia assay when administered as a single 10 mg/kg subcutaneous dose.

TABLE 14
Single subcutaneous 10 mg/kg dose on Day 7; measurement 1 hour post dose

| | | | Inhibition | | Inhibition | | Inhibition |
|---|---|---|---|---|---|---|---|
| | Inhibition of | | of | | of | | of |
| | Allodynia | | Allodynia | | Allodynia | | Allodynia |
| Ex. No. | (%) | Ex. No. | (%) | Ex. No. | (%) | Ex. No. | (%) |
| 4 | 43 | 6 | 16 | 8 | 22 | 11 | 9 |
| 12 | 88 | 14 | 48 | 23 | 65 | 25 | 77 |
| 29 | 11 | 44 | 18 | 45 | 1.0 | — | — |

Alternatively, the animals may be subcutaneously dosed according to the above protocol except with 30 mg/kg of test compound. For animals dosed with the 30 mg/kg of test compound, the contralateral (i.e., left hind paw) PWT values are determined at 2 hours after the single dose. Additional compounds such as the compounds of Examples 6, 8, 29, and 44 may show activity (i.e., greater than 30% inhibition) in this assay when dosed at 30 mg/kg.

Alternatively, the animals may be orally dosed according to the above protocol with 10 mg/kg (or 30 mg/kg) of test compound. For oral dosing the vehicle is phosphate buffered saline containing 0.5% hydroxy-propylmethylcellulose (HPMC) and 0.2% TWEEN™ 80 and PWT values are determined at 2 hours after the single dose.

Alternatively in any of the protocols, PWT values are determined at about the time corresponding to the estimated Cₘₐₓ of the test compound, as determined by one of ordinary skill in the art.

The compounds and pharmaceutically acceptable acid addition salts thereof of the invention inhibit binding of norepinephrine and serotonin, and inhibit capsaicin-induced mechanical allodynia in rats, a model of neuropathic pain, including the pain of fibromyalgia.

The compounds and salts are effective for treating diseases and disorders such as depression, generalized anxiety disorder, attention deficit hyperactivity disorder (ADHD), fibromyalgia, neuropathic pain, urinary incontinence, and schizophrenia.

All publications, patents, patent applications, and patent application publications cited herein are hereby incorporated by reference in their entirety for all purposes. Examples used to illustrate embodiments do not limit the invention.

1. A compound of Formula (I) 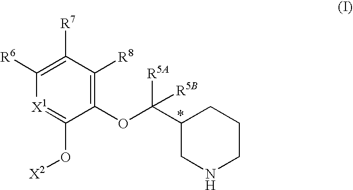
or a pharmaceutically acceptable acid addition salt thereof,
wherein:
* indicates a first chiral carbon atom;
R^{5A} and R^{5B} independently are H, (C₁-C₄)alkyl, phenyl, or pyridyl;
X¹ is N or C—R¹;
R¹ is H or halo;
R⁶ independently is H, halo, (C₁-C₄)alkyl, or —O(C₁-C₄)alkyl;
R⁷ and R⁸ independently are H or F;
X² is 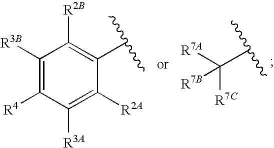
R^{2A}, R^{2B}, R^{3A}, R^{3B}, and R⁴ independently are H, halo, (C₁-C₄)alkyl, —CN, or —O(C₁-C₄)alkyl, or R^{2A} and R^{3A}, or R^{3A} and R⁴, may be taken together with the carbons to which they are attached to form a 1,2-cyclopentenylene or 1,2-cyclohexenylene;
R^{7A} and R^{7B} independently are H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl, or R^{7A} and R^{7B} optionally may be taken together with the carbon to which they are attached to form a (C₃-C₆)cycloalkyl;
R^{7C} is H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl;
each of the 1,2-cyclopentenylene, 1,2-cyclohexenylene, (C₁-C₄)alkylene, (C₁-C₄)alkyl, (C₃-C₆)cycloalkyl, and —O(C₁-C₄)alkyl independently is unsubstituted or substituted with from 1 to 5 substituents R^{S};
each phenyl independently is unsubstituted or substituted with from 1 to 5 substituents R^{T};
each pyridyl is unsubstituted or substituted with from 1 to 4 substituents R^{T};
each R^{S} independently is F, —CH₃, —CF₃, —CN, —OCH₃, ═O, —NH₂, —N(H)CH₃, or —N(CH₃)₂;
each R^{T} independently is F, Cl, —CH₃, —CF₃, —CN, —OCH₃, —OCH₂CH₃, —NH₂, or —N(H)CH₃; and
wherein at least one of R¹, R^{2A}, R^{2B}, R^{3A}, R^{3B}, R⁴, R⁶, R⁷, and R⁸ is not H; and X² is not —CH₃.

2. A compound according to claim 1, or a pharmaceutically acceptable acid addition salt thereof, wherein:
X² is 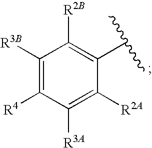
one of R^{2A}, R^{2B}, R^{3A}, and R⁴ is halo, (C₁-C₄)alkyl, or —O(C₁-C₄)alkyl; and the remainder of R^{2A}, R^{2B}, R^{3A}, R^{3B}, and R⁴ independently are H, halo, (C₁-C₄)alkyl, or —O(C₁-C₄)alkyl.

3. A compound according to claim 1, or a pharmaceutically acceptable acid addition salt thereof, wherein:
X² is 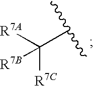
and R^{7A}, R^{7B}, and R^{7C} independently are H, F, (C₁-C₄)alkyl, (C₃-C₆)cycloalkyl, —(C₁-C₄)alkylene-(C₃-C₆)cycloalkyl, phenyl, or —(C₁-C₄)alkylene-phenyl; and X² is not —CH₃.

4. A compound according to claim 1, or a pharmaceutically acceptable acid addition salt thereof, wherein:
X² is 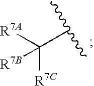
R^{7A} and R^{7B} are taken together with the carbon to which they are attached to form a (C₃-C₆)cycloalkyl; and R^{7C} is H.

5. A compound according to claim 1, or a pharmaceutically acceptable acid addition salt thereof, wherein X¹ is N and R⁶ is H or —CH₃.

6. A compound according to claim 1, or a pharmaceutically acceptable acid addition salt thereof, wherein X¹ is C—R¹; R¹ is H or F; and R⁶ is H, F, Cl, —CH₃, —CF₃, —OCF₃, or —OCH₃.

7. A compound according to claim 1, or a pharmaceutically acceptable acid addition salt thereof, wherein R^{5A} and R^{5B} are each H.

8. A compound according to claim 1, or a pharmaceutically acceptable acid addition salt thereof, wherein R^{5A} is unsubstituted (C₁-C₄)alkyl, unsubstituted phenyl, or unsubstituted pyridyl; R^{5B} is H; and the carbon to which R^{5A} and R^{5B} are attached is a second chiral carbon atom.

9. A compound according to claim 1, or a pharmaceutically acceptable acid addition salt thereof, wherein the stereochemistry is (S) at the first chiral carbon atom.

10. A compound according to claim 1, wherein the compound is selected from the group consisting of:
(S)-2-(2-methoxy-4-methyl-phenoxy)-6-methyl-3-(piperidin-3-ylmethoxy)-pyridine;
(S)-2-(4-chloro-2-methoxy-phenoxy)-6-methyl-3-(piperidin-3-ylmethoxy)-pyridine;
(S)-2-(2-chloro-4-methyl-phenoxy)-6-methyl-3-(piperidin-3-ylmethoxy)-pyridine;
(S)-2-(4-chloro-2-fluoro-phenoxy)-6-methyl-3-(piperidin-3-ylmethoxy)-pyridine;
(S)-2-(2,4-difluoro-phenoxy)-6-methyl-3-(piperidin-3-ylmethoxy)-pyridine;
(S)-6-methyl-3-(piperidin-3-ylmethoxy)-2-p-tolyloxy-pyridine;
(S)-2-(4-ethyl-2-methoxy-phenoxy)-6-methyl-3-(piperidin-3-ylmethoxy)-pyridine;
(S)-2-(4-chloro-phenoxy)-6-methyl-3-(piperidin-3-ylmethoxy)-pyridine;
(S)-2-(3-chloro-phenoxy)-6-methyl-3-(piperidin-3-ylmethoxy)-pyridine;
(S)-2-(3,4-dichloro-phenoxy)-6-methyl-3-(piperidin-3-ylmethoxy)-pyridine;
(S)-2-(2,4-dichloro-phenoxy)-6-methyl-3-(piperidin-3-ylmethoxy)-pyridine;
(S)-2-(2-chloro-4-fluoro-phenoxy)-6-methyl-3-(piperidin-3-ylmethoxy)-pyridine;
(S)-2-(4-chloro-3-fluoro-phenoxy)-6-methyl-3-(piperidin-3-ylmethoxy)-pyridine;
(S)-3-[4-chloro-2-(4-chloro-2-fluoro-phenoxy)-phenoxymethyl]-piperidine;
(S)-3-[4-chloro-2-(4-chloro-2-methoxy-phenoxy)-phenoxymethyl]-piperidine;
(S)-3-[2-(4-chloro-2-methoxy-phenoxy)-4-trifluoromethyl-phenoxymethyl]-piperidine; and
(S)-3-[4-chloro-2-(2-fluoro-6-methoxy-phenoxy)-phenoxymethyl]-piperidine;
or a pharmaceutically acceptable acid addition salt thereof.

11. A compound according to claim 1, wherein the compound is selected from the group consisting of:
(S)-2-(4-fluoro-phenoxy)-6-methyl-3-(piperidin-3-ylmethoxy)-pyridine;
(S)-2-(4-fluoro-phenoxy)-3-(piperidin-3-ylmethoxy)-pyridine;
(S)-2-(2,4-difluoro-phenoxy)-3-(piperidin-3-ylmethoxy)-pyridine;
(S)-2-(3-chloro-phenoxy)-3-(piperidin-3-ylmethoxy)-pyridine;
(S)-2-(3,4-difluoro-phenoxy)-3-(piperidin-3-ylmethoxy)-pyridine;
(S)-2-(2-chloro-4-fluoro-phenoxy)-3-(piperidin-3-ylmethoxy)-pyridine;
(S)-2-(2,6-difluoro-phenoxy)-3-(piperidin-3-ylmethoxy)-pyridine;
(S,S)-2-phenoxy-3-(1-piperidin-3-yl-propoxy)-pyridine;
(S)-2-ethoxy-3-(phenyl-piperidin-3-yl-methoxy)-pyridine, stereoisomer A;
(S)-2-phenoxy-3-(piperidin-3-ylmethoxy)-pyridine;
(S)-6-methyl-2-phenoxy-3-(piperidin-3-ylmethoxy)-pyridine;
(S)-3-(2-phenoxy-phenoxymethyl)-piperidine;
(S)-3-(4-fluoro-2-phenoxy-phenoxymethyl)-piperidine;
(S)-3-[(S)-1-(2-benzyloxy-phenoxy)-ethyl]-piperidine;
(S)-3-[(S)-1-(2-isobutoxy-phenoxy)-ethyl]-piperidine;
(S)-3-[(S)-1-(2-cyclobutylmethoxy-phenoxy)-ethyl]-piperidine;
(S)-3-[(S)-1-(2-cyclohexyloxy-phenoxy)-ethyl]piperidine;
(S)-3-[2-fluoro-6-(4-fluoro-phenoxy)-phenoxymethyl]-piperidine;
(S)-3-[2-(3,4-difluoro-phenoxy)-6-fluoro-phenoxymethyl]-piperidine;
(S)-3-[3-fluoro-2-(4-fluoro-phenoxy)-phenoxymethyl]-piperidine;
2-[{(R)-2-fluoro-6-methoxy-phenoxy}-(S)-piperidin-3-yl-methyl]-pyridine; and
2-[(S)-piperidin-3-yl-{(R)-2-trifluoromethoxy-phenoxy}-methyl]-pyridine;
or a pharmaceutically acceptable acid addition salt thereof.

12. A pharmaceutical composition comprising a compound according to claim 1, or a pharmaceutically acceptable acid addition salt thereof, and a pharmaceutically acceptable excipient.

13-15. (canceled)

16. A method of treating fibromyalgia, the method comprising administering to a patient in need of such treatment a therapeutically effective amount of a compound according to claim 1, or a pharmaceutically acceptable acid addition salt thereof.

17. A method of treating osteoarthritis or rheumatoid arthritis, the method comprising administering to a patient in need of such treatment a therapeutically effective amount of a compound according to claim 1, or a pharmaceutically acceptable acid addition salt thereof.

18. A method of treating a disease or disorder selected from the group consisting of: attention deficit hyperactivity disorder; neuropathic pain; anxiety; depression; and schizophrenia, the method comprising administering to a patient in need of such treatment a therapeutically effective amount of a compound according to claim 1, or a pharmaceutically acceptable acid addition salt thereof.
